(19) United States
(12) Patent Application Publication
(10) Pub. No.: US 2008/0261990 A1
(43) Pub. Date: Oct. 23, 2008

(54) Novel Pyrimidine Derivatives and their Use

(75) Inventors: Elke Dittrich-Wengenroth, Wuppertal (DE); Lars Barfacker, Oberhausen (DE); Axel Kretschmer, Wuppertal (DE); Claudia Hirth-Dietrich, Wuppertal (DE); Peter Ellinghaus, Melle (DE); Martin Raabe, Ulm (DE); Hilmar Bischoff, Wuppertal (DE); Christian Pilger, Ludwigshafen (DE); Ulrich Rosentreter, Wunstorf (DE); Stephan Bartel, Kurten (DE); Klemens Lustig, Wuppertal (DE); Armin Kern, Wuppertal (DE); Dieter Lang, Velbert (DE); Marcus Bauser, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer healthCare AG, Leverkusen (DE)

(21) Appl. No.: 11/663,813

(22) PCT Filed: Sep. 10, 2005
(86) PCT No.: PCT/EP2005/009734

§ 371 Date: Feb. 28, 2008

(30) Foreign Application Priority Data
Sep. 25, 2004 (DE) 10 2004 046 623.8

(51) Int. Cl. A61K 31/496; C07D 405/12; A61K 31/506; C07D 239/28; A61K 31/505

(52) U.S. Cl. 514/252.14; 544/333; 514/256; 544/329

(57) ABSTRACT

The present invention relates to novel pyrimidine derivatives, to processes for their preparation, to their use for the treatment and/or prophylaxis of diseases and to their use for preparing medicaments for the treatment and/or prophylaxis of diseases, preferably for the treatment and/or prevention of cardiovascular diseases, in particular dyslipidemias, arteriosclerosis, coronary heart disease, thrombosis and metabolic syndrome.

The present invention relates to novel pyrimidine derivatives, to processes for their preparation, to their use for the treatment and/or prophylaxis of diseases and to their use for preparing medicaments for the treatment and/or prophylaxis of diseases, preferably for the treatment and/or prevention of cardiovascular diseases, in particular dyslipidemias, arteriosclerosis, coronary heart disease, thrombosis and metabolic syndrome.

In spite of many successful therapies, cardiovascular disorders remain a serious public health problem. Treatment with statins, which inhibit HMG-CoA reductase, very successfully lowers both LDL cholesterol (LDL-C) plasma concentrations and the mortality of patients at risk; however, convincing treatment strategies for the therapy of patients having an unfavorable HDLC/LDL-C ratio and/or hypertriglyceridemia are still not available to date.

Currently, in addition to niacin, fibrates are the only therapy option for patients of these risk groups. They lower elevated triglyceride levels by 20-50%, reduce LDL-C by 10-15%, change the LDL particle size of atherogenic LDL of low density to less atherogenic LDL of normal density and increase the HDL concentration by 10-15%.

Fibrates act as weak agonists of the peroxysome-proliferator-activated receptor (PPAR)-alpha (Nature 1990, 347, 645-50). PPAR-alpha is a nuclear receptor which regulates the expression of target genes by binding to DNA sequences in the promoter range of these genes [also referred to as PPAR response elements (PPRE)]. PPREs have been identified in a number of genes coding for proteins which regulate lipid metabolism. PPAR-alpha is highly expressed in the liver, and its activation leads inter alia to lower VLDL production/secretion and reduced apolipoprotein CIII (ApoCIII) synthesis. In contrast, the synthesis of apolipoprotein A1 (ApoA1) is increased.

A disadvantage of fibrates which have hitherto been approved is that their interaction with the receptor is only weak (EC₅₀ in the μM range), which in turn is responsible for the relatively small pharmacological effects described above.

It was an object of the present invention to provide novel compounds suitable for use as PPAR-alpha modulators for the treatment and/or prevention of in particular cardiovascular disorders.

WO 03/074495, WO 2005/040102 and US 2005/0096337-A1 claim various phenoxy- and/or phenylthioacetic acid derivatives as PPAR modulators. DE 42 39 440-A1 describes 4-aminopyrimidine derivatives and their use for treating hypertension and myocardial insufficiency. EP 0 539 066-A1 discloses similar heterocyclic compounds for the same applications. WO 03/063794 claims 2,4-diaminopyrimidine derivatives as inhibitors of the IgE and/or IgG receptor signal cascade.

The present invention provides compounds of the general formula (I)

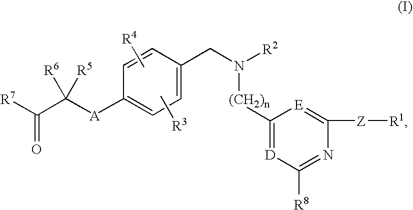

in which
A represents O or S,
   one of the ring members D and E represents N and the other represents CH,
Z represents (CH₂)ₘ, O or N—R⁹, where
   m represents the number 0, 1 or 2,
   and
   R⁹ represents hydrogen or (C₁-C₆)-alkyl,
n represents the number 0, 1 or 2,
R¹ represents (C₆-C₁₀)-aryl or 5- to 10-membered heteroaryl which may in each case be substituted up to four times by identical or different substituents selected from the group consisting of halogen, nitro, cyano, (C₁-C₆)-alkyl (which for its part may be substituted by hydroxyl), (C₃-C₈)-cycloalkyl, phenyl, hydroxyl, (C₁-C₆)-alkoxy, trifluoromethyl, trifluoromethoxy, amino, mono- and di-(C₁-C₆)-alkylamino, R¹⁰—C(O)—NH—, R¹¹—C(O)—, R¹²R¹³N—C(O)—NH— and R¹⁴R¹⁵N—C(O)—, where
   R¹⁰ represents hydrogen, (C₁-C₆)-alkyl, (C₃-C₈)-cycloalkyl, phenyl or (C₁-C₆)-alkoxy,
   R¹¹ represents hydrogen, (C₁-C₆)-alkyl, (C₃-C₈)-cycloalkyl, phenyl, hydroxyl or (C₁-C₆)-alkoxy
   and
   R¹², R¹³, R¹⁴ and R¹⁵ are identical or different and independently of one another represent hydrogen, (C₁-C₆)-alkyl, (C₃-C₈)-cycloalkyl or phenyl,
      or
R¹ represents (C₃-C₇)-cycloalkyl or a 5- or 6-membered heterocycle which may in each case be substituted up to two times by identical or different substituents from the group consisting of (C₁-C₆)-alkyl, (C₁-C₆)-alkoxy, trifluoromethyl or trifluoromethoxy,
   or
   the grouping -Z-R¹ represents a group of the formula

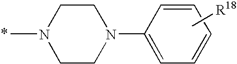

in which
R¹⁸ represents hydrogen, halogen, (C₁-C₆)-alkyl, (C₁-C₆)-alkoxy, trifluoromethyl or trifluoromethoxy
and
* represents the point of attachment,
R² represents hydrogen, (C₆-C₁₀)-aryl, (C₁-C₆)-alkyl, (C₂-C₆)-alkenyl or (C₂-C₆)-alkynyl, where alkyl, alkenyl and alkynyl may in each case be substituted by trifluoromethyl, (C₁-C₆)-alkoxy, trifluoromethoxy, fluorine, cyano, (C₃-C₆)-cycloalkyl, (C₆-C₁₀)-aryl or 5- or 6-membered heteroaryl, where all aryl and heteroaryl groups mentioned for their part may be substituted up to three times by identical or different substituents selected from the group consisting of halogen, nitro, cyano, (C₁-C₆)-alkyl, hydroxyl, (C₁-C₆)-alkoxy, trifluoromethyl and trifluoromethoxy,
R³ and R⁴ are identical or different and independently of one another represent hydrogen, (C₁-C₆)-alkyl, (C₂-C₆)-alkenyl, (C₁-C₆)-alkoxy, trifluoromethyl, trifluoromethoxy or halogen,
R⁵ and R⁶ are identical or different and independently of one another represent hydrogen, (C₁-C₆)-alkyl, (C₁-C₆)-alkoxy or phenoxy or together with the carbon atom to which they are attached form a (C₃-C₈)-cycloalkyl ring,
R⁷ represents a group of the structure —NHR¹⁶ or —OR¹⁷, in which
   R¹⁶ represents hydrogen, (C₁-C₆)-alkyl or (C₁-C₆)-alkylsulfonyl
   and
   R¹⁷ represents hydrogen or represents a hydrolysable group which can be converted into the corresponding carboxylic acid,
      and
   R⁸ represents hydrogen or (C₁-C₆)-alkyl,
      and their salts, solvates and solvates of the salts.

In the context of the invention, in the definition of R¹⁷, a hydrolysable group means a group which, in particular in the body, causes the —C(O)OR¹⁷ grouping to be converted into the corresponding carboxylic acid (R¹⁷=hydrogen). Such groups are, by way of example and by way of preference, benzyl, (C₁-C₆)-alkyl or (C₃-C₈)-cycloalkyl which are in each case optionally mono- or polysubstituted by identical or different substituents from the group consisting of halogen, hydroxyl, amino, (C₁-C₆)-alkoxy, carboxyl, (C₁-C₆)-alkoxycarbonyl, (C₁-C₆)-alkoxycarbonylamino or (C₁-C₆)-alkanoyloxy, or, in particular, (C₁-C₄)-alkyl which is optionally mono- or disubstituted by identical or different substituents from the group consisting of halogen, hydroxyl, amino, (C₁-C₄)-alkoxy, carboxyl, (C₁-C₄)-alkoxycarbonyl, (C₁-C₄)-alkoxycarbonylamino or (C₁-C₄)-alkanoyloxy.

Compounds of the invention are the compounds of the formula (I) and their salts, solvates and solvates of the salts, the compounds, comprised by formula (I), of the formulae mentioned below and their salts, solvates and solvates of the salts and the compounds, comprised by the formula (I), mentioned below as embodiments and their salts, solvates and solvates of the salts if the compounds, comprised by formula (I), mentioned below are not already salts, solvates and solvates of the salts.

Depending on their structure, the compounds of the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds of the invention can be present in tautomeric forms, the present invention comprises all tautomeric forms.

In the context of the present invention, preferred salts are physiologically acceptable salts of the compounds of the invention. The invention also comprises salts which for their part are not suitable for pharmaceutical applications, but which can be used, for example, for isolating or purifying the compounds of the invention.

Physiologically acceptable salts of the compounds of the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalene disulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of customary bases, such as, by way of example and by way of preference, alkali metal salts (for example sodium salts and potassium salts), alkaine earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds of the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds of the invention. The term “prodrugs” includes compounds which for their part may be biologically active or inactive but which, during the time they spend in the body, are converted into compounds of the invention (for example metabolically or hydrolytically).

In the context of the present invention, unless specified differently, the substituents have the following meanings:

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, iso-butyl, sec-butyl, tert-butyl, 1-ethylpropyl, n-pentyl and n-hexyl.

In the context of the invention, (C₂-C₆)-alkenyl and (C₁-C₄)-alkenyl represent a straight-chain or branched alkenyl radical having 2 to 6 and 2 to 4 carbon atoms, respectively. Preference is given to a straight-chain or branched alkenyl radical having 2 to 4 carbon atoms. The following radicals may be mentioned by way of example and by way of preference: vinyl, allyl, isopropenyl, n-but-2-en-1-yl and 2-methyl-2-propen-1-yl.

In the context of the invention, C₂-C₆)-alkynyl and (C₂-C₄)-alkynyl represent a straight-chain or branched alkynyl radical having 2 to 6 and 2 to 4 carbon atoms, respectively. Preference is given to a straight-chain or branched alkynyl radical having 2 to 4 carbon atoms. The following radicals may be mentioned by way of example and by way of preference: ethynyl, n-prop-2-yn-1-yl, n-but-2-yn-1-yl and n-but-3-yn-1-yl.

In the context of the invention, C₃-C₈)-cycloalkyl, (C₃-C₇)-cycloalkyl and (C₃-C₆)-cycloalkyl represent a mono- or, if appropriate, bicyclic cycloalkyl group having 3 to 8, 3 to 7 and 3 to 6 carbon atoms, respectively. Preference is given to a cycloalkyl radical having 3 to 6 carbon atoms. The following radicals may be mentioned by way of example and by way of preference: cyclopropyl, cyclobutyl, cyclopentyl, cyclohexyl and cycloheptyl.

In the context of the invention, (C₆-C₁₀ represents an aromatic radical having preferably 6 to 10 carbon atoms. Preferred aryl radicals are phenyl and naphthyl.

In the context of the invention, (C₁-C₆)-alkoxy and (C₁-C₄)-alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and tert-butoxy.

In the context of the invention, (C₁-C₆)-alkoxycarbonyl and (C₁-C₄)-alkoxycarbonyl represent a straight-chain or branched alkoxy radical having 1 to 6 and 1 to 4 carbon atoms, respectively, which is attached via a carbonyl group. Preference is given to a straight-chain or branched alkoxycarbonyl radical having 1 to 4 carbon atoms in the alkoxy group. The following radicals may be mentioned by way of example and way of preference: methoxycarbonyl, ethoxycarbonyl, n-propoxycarbonyl, isopropoxycarbonyl and tert-butoxycarbonyl.

In the context of the invention, (C₁-C₆)-alkylsulfonyl represents a straight-chain or branched alkylsulfonyl radical having 1 to 6 carbon atoms. Preference is given to a straight-chain or branched alkylsulfonyl radical having 1 to 4 carbon atoms. The following radicals may be mentioned by way of example and by way of preference: methylsulfonyl, ethylsulfonyl, n-propylsulfonyl, isopropylsulfonyl, n-butylsulfonyl and tert-butylsulfonyl.

In the context of the invention, mono-(C₁-C₆)-alkylamino and mono-(C₁-C₄)-alkylamino represent an amino group having a straight-chain or branched alkyl substituent which has 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tertbutylamino.

In the context of the invention, di-(C₁-C₆)-alkylamino and di-(C₁-C₄)-alkylamino represent an amino group having two identical or different straight-chain or branched alkyl substituents which have in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₁-C₆)-alkoxycarbonylamino and (C₁-C₄)-alkoxycarbonylamino represent an amino group having a straight-chain or branched alkoxycarbonyl substituent which has 1 to 6 and 1 to 4 carbon atoms, respectively, in the alkoxy radical and is attached to the nitrogen atom via the carbonyl group. Preference is given to an alkoxycarbonylamino radical having 1 to 4 carbon atoms. The following radicals may be mentioned by way of example and by way of preference: methoxycarbonylamino, ethoxycarbonylamino, n-propoxycarbonylamino, isopropoxycarbonylamino and tert-butoxycarbonylamino.

In the context of the invention, (C₁-C₆)-alkanoyloxy and (C₁-C₄)-alkanoyloxy represent a straight-chain or branched alkyl radical having 1 to 6 and 1 to 4 carbon atoms, respectively, which carries a doubly attached oxygen atom in the 1-position and is attached in the 1-position via a further oxygen atom. Preference is given to an alkanoyloxy radical having 1 to 4 carbon atoms. The following radicals may be mentioned by way of example and by way of preference: acetoxy, propionoxy, n-butyroxy, i-butyroxy, pivaloyloxy and n-hexanoyloxy.

In the context of the invention, 5- to 10-membered heteroaryl represents a mono- or, if appropriate, bicyclic aromatic heterocycle (heteroaromatic) having up to four identical or different heteroatoms from the group consisting of N, O and/or S which is attached via a ring carbon atom or, if appropriate, via a ring nitrogen atom of the heteroaromatic. The following radicals may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl. Preference is given to monocyclic 5- or 6-membered heteroaryl radicals having up to three heteroatoms from the group consisting of N, O and/or S, such as, for example, furyl, thienyl, thiazolyl, oxazolyl, isothiazolyl, isoxazolyl, pyrazolyl, imidazolyl, triazolyl, oxadiazolyl, thiadiazolyl, pyridyl, pyrimidinyl, pyridazinyl, pyrazinyl, triazinyl.

In the context of the invention, a 5- or 6-membered heterocycle represents a straight-chain heterocycle having a total of 5 and 6 ring atoms, respectively, which contains one or two heteroatoms from the group consisting of N, O and/or S in the ring. The following radicals may be mentioned by way of example: tetrahydrofuryl, tetrahydrothienyl, tetrahydropyranyl, tetrahydrothiopyranyl, pyrrolidinyl, oxazolidinyl, thiazolidinyl, imidazolidinyl, piperidinyl, piperazinyl, morpholinyl and thiomorpholinyl. Preference is given to tetrahydrofuryl and tetrahydropyranyl.

In the context of the invention, halogen includes fluorine, chlorine, bromine and iodine. Preference is given to chlorine or fluorine.

If radicals in the compounds of the invention are substituted, the radicals can, unless specified otherwise, be mono- or polysubstituted. In the context of the present invention, the meanings of radicals which occur more than once are independent of one another. Substitution with one, two or three identical or different substituents is preferred. Very particular preference is given to substitution with one substituent.

In the context of the present invention, preference is given to compounds of the formula (I) in which
A represents O or S,
   one of the ring members D and E represents N and the other represents CH,
Z represents (CH₂)ₘ, O or NH, where
   m represents the number 0 or 1,
n represents the number 0 or 1,
R¹ represents phenyl or 5- or 6-membered heteroaryl which may in each case be substituted up to four times by identical or different substituents selected from the group consisting of halogen, nitro, cyano, (C₁-C₄)-alkyl (which for its part may be substituted by hydroxyl), (C₃-C₆)-cycloalkyl, phenyl, hydroxyl, (C₁-C₄)-alkoxy, trifluoromethyl, trifluoromethoxy, amino, mono- and di-(C₁-C₄)-alkylamino, R¹⁰—C(O)—NH—, R¹¹—C(O)—, R¹²R¹³N—C(O)—NH— and R¹⁴R¹⁵N—C(O)—, where
   R¹⁰ represents hydrogen, (C₁-C₄)-alkyl, (C₃-C₆)-cycloalkyl, phenyl or (C₁-C₄)-alkoxy,
   R¹¹ represents hydrogen, (C₁-C₄)-alkyl, (C₃-C₆)-cycloalkyl, phenyl, hydroxyl or (C₁-C₄)-alkoxy
   and
   R¹², R¹³, R¹⁴ and R¹⁵ are identical or different and independently of one another represent hydrogen, (C₁-C₄)-alkyl, (C₃-C₆)-cycloalkyl or phenyl,
      or
R¹ represents cyclohexyl or 4-tetrahydropyranyl which may in each case be substituted up to two times by identical or different substituents from the group consisting of (C₁-C₄)-alkyl, (C₁-C₄)-alkoxy and trifluoromethyl,
R² represents hydrogen, phenyl, (C₁-C₄)-alkyl, (C₂-C₄)-alkenyl or (C₂-C₄)-alkynyl, where alkyl, alkenyl and alkynyl may in each case be substituted by trifluoromethyl, fluorine, cyano, (C₁-C₄)-alkoxy, cyclopropyl, cyclobutyl, phenyl or a 5- or 6-membered heteroaryl, where all phenyl and heteroaryl groups mentioned for their part may in each case be substituted up to three times by identical or different substituents selected from the group consisting of halogen, nitro, cyano, (C₁-C₄)-alkyl, hydroxyl, (C₁-C₄)-alkoxy, trifluoromethyl and trifluoromethoxy,
R³ and R⁴ are identical or different and independently of one another represent hydrogen, (C₁-C₄)-alkyl, (C₁-C₄)-alkoxy, trifluoromethyl, trifluoromethoxy or halogen,
R⁵ and R⁶ are identical or different and independently of one another represent hydrogen, methyl, ethyl, methoxy, ethoxy or phenoxy or together with the carbon atom to which are attached form a (C₃-C₆)-cycloalkyl ring,
R⁷ represents a group of the formula —NHR¹⁶ or —OR¹⁷, in which
   R¹⁶ represents hydrogen or —(C₁-C₄)-alkyl
   and
   R¹⁷ represents hydrogen or represents a hydrolysable group which may be converted into the corresponding carboxylic acid,
      and
R⁸ represents hydrogen or methyl,
   and their salts, solvates and solvates of the salts.

In the context of the present invention, particular preference is given to compounds of the formula (I) in which
A represents S,
   one of the ring members D and E represents N and the other represents CH,
Z represents (CH₂)ₘ, O or NH, where
   m represents the number 0 or 1,
n represents the number 0 or 1,
R¹ represents phenyl or pyridyl which may in each case be mono- or disubstituted by identical or different substituents from the group consisting of fluorine, chlorine, nitro, methyl, methoxy, trifluoromethyl and trifluoromethoxy
   or
R¹ represents cyclohexyl which may be substituted in the 4-position by methyl or methoxy,
R² represents hydrogen, propargyl or represents (C₁-C₄)-alkyl which may be substituted by fluorine, cyano, (C₁-C₄)-alkoxy, cyclopropyl, phenyl, furyl, thienyl, imidazolyl, oxazolyl, isoxazolyl, thiazolyl, oxadiazolyl or thiadiazolyl, where phenyl and all heteroaromatic rings mentioned for their part may in each case be mono- or disubstituted by identical or different substituents selected from the group consisting of fluorine, chlorine, methyl, ethyl, isopropyl, tert-butyl, methoxy, ethoxy, trifluoromethyl and trifluoromethoxy,
R³ and R⁴ are identical or different and independently of one another represent hydrogen, methyl, methoxy, fluorine or chlorine,
R⁵ and R⁶ are identical or different and represent hydrogen or methyl,
R⁷ represents —OH, —NH₂ or —NHCH₃,
   and
R⁸ represents hydrogen,
   and their salts, solvates and solvates of the salts.

Of particular importance are compounds of the general formula (I-A)

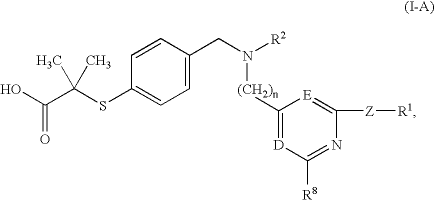

in which

R¹, R², R⁸, D, E, Z and n are each as defined above,

and their salts, solvates and solvates of the salts.

Of very particular importance are compounds of the general formula (I-C)

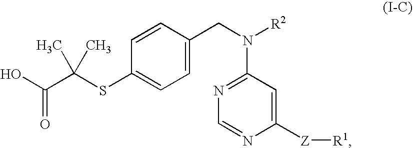

in which

Z represents a bond or represents O

and

R¹ and R² are each as defined above, and their salts, solvates and solvates of the salts.

The individual radical definitions given in the respective combinations or preferred combinations of radicals may, independently of the particular given combination of the radicals, also be replaced by any radical definitions of other combinations.

Very particular preference is given to combinations of two or more of the preferred ranges mentioned above.

The invention furthermore provides a process for preparing the compounds of the formulae (I), (I-A) or (I-C) according to the invention, characterized in that compounds of the formula (II)

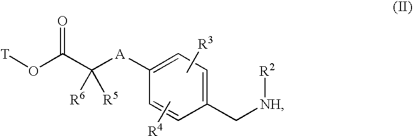

in which R², R³, R⁴, R⁵, R⁶ and A are each as defined above
and

T represents (C₁-C₄)-alkyl, preferably tert-butyl, or represents benzyl,

are either
[A] initially reacted in an inert solvent in the presence of a base with a compound of the formula (III)

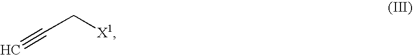

in which
X¹ represents a suitable leaving group, such as, for example, halogen,
to give compounds of the formula (IV)

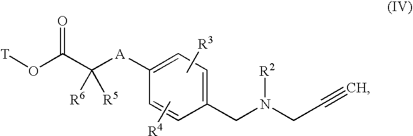

in which A, T, R², R³, R⁴, R⁵ and R⁶ are each as defined above,
then converted, in an inert solvent in the presence of copper(I) iodide, a suitable palladium catalyst and a base, with a compound of the formula (V)

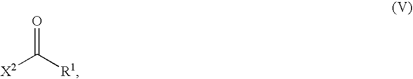

in which R¹ is as defined above and
X² represents a suitable leaving group, such as, for example, halogen,
into compounds of the formula (VI)

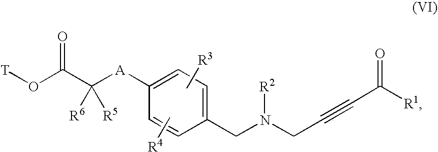

in which A, T, R², R², R³, R⁴, R⁵ and R⁶ are each as defined above,
which compounds are then reacted, in an inert solvent in the presence of a base, with a compound of the formula (VII)

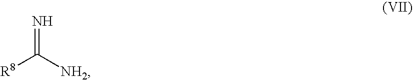

in which R⁸ is as defined above,
to give compounds of the formula (VIII)

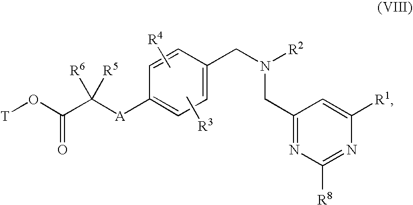

in which A, T, R¹, R², R³, R⁴, R⁵, R⁶ and R⁸ are each as defined above,
   or
[B] initially converted, in an inert solvent in the presence of a base, with a compound of the formula (IX)

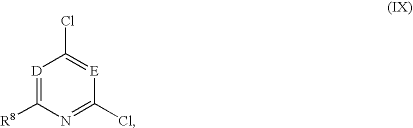

in which D, E and R⁸ are each as defined above,
into compounds of the formula (X)

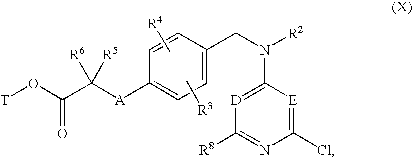

in which A, D, E, T, R², R³, R⁴, R⁵, R⁶ and R⁸ are each as defined above,
and these compounds are then either
[B-1]
reacted, in an inert solvent in the presence of a base, with a compound of the formula (XI)

R¹-Z¹-H (XI),
in which R¹ is as defined above and
Z¹ represents O or N—R⁹, where R⁹ is as defined above,
to give compounds of the formula (XII)

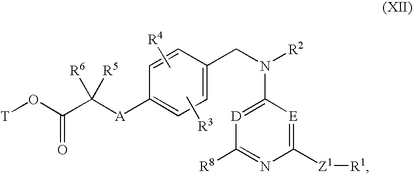

in which A, D, E, T, Z¹, R¹, R², R³, R⁴, R⁵, R⁶ and R⁸ are each as defined above,
or
[B-2]
reacted, in an inert solvent in the presence of a palladium catalyst and a base, with a compound of the formula (XIII)

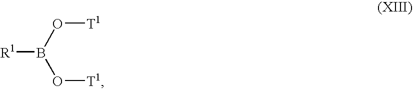

in which R¹ is as defined above and
T¹ represents hydrogen or (C₁-C₄)-alkyl,
to give compounds of the formula (XIV)

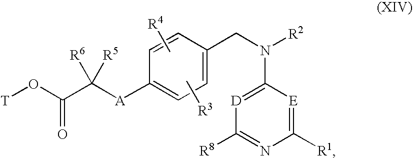

in which A, D, E, T, R¹, R², R³, R⁴, R⁵, R⁶ and R⁸ are each as defined above,
or
[B-3]
reacted, in an inert solvent in the presence of a palladium catalyst, with a compound of the formula (XV)

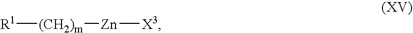

in which m and R¹ are each as defined above and
X³ represents halogen, in particular bromine,
to give compounds of the formula (XVI)

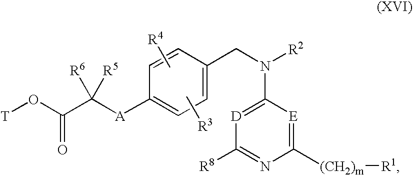

in which m, A, D, E, T, R¹, R², R³, R⁴, R⁵, R⁶ and R⁸ are each as defined above,
   or
[C] reacted, in an inert solvent in the presence of a base, with a compound of the formula (XVII)

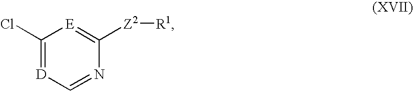

in which D, E and R¹ are each as defined above and
Z² represents a bond, O or N—R⁹, where R⁹ is as defined above,
to give compounds of the formula (XVIII)

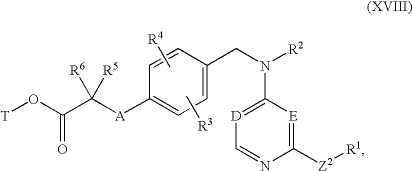

in which A, D, E, T, Z², R¹, R², R³, R⁴, R⁵ and R⁶ are each as defined above,
   and the resulting compounds of the formulae (VIII), (XII), (XIV), (XVI) and (XVIII) are subsequently converted by basic or acidic hydrolysis or, if T represents benzyl, also hydrogenolytically, into the respective carboxylic acids of the formula (I-B)

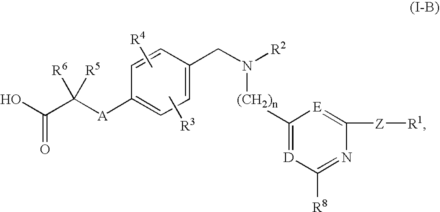

in which n, A, D, E, Z, R¹, R², R³, R⁴, R⁵, R⁶ and R⁸ are each as defined above,
and, if appropriate, subsequently converted into the compounds of the formula (I) using esterification or amidation methods known from the literature,
and the compounds of the formula (I) are, if appropriate, reacted with the appropriate (i) solvents and/or (ii) bases or acids to give their solvates, salts and/or solvates of the salts.

The compounds of the formula (II) and their preparation are described in WO 02/28821 or can be prepared analogously to the processes described therein. Compounds of the formula (II) in which

A represents S can also be prepared by initially converting compounds of the formula (XIX)

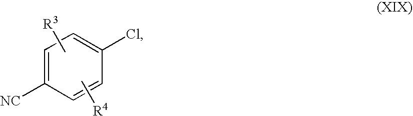

in which R³ and R⁴ are each as defined above in an inert solvent with sodium sulfide into compounds of the formula (XX)

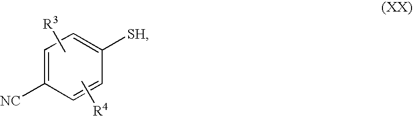

in which R³ and R⁴ are each as defined above,
reacting these subsequently with or without intermediate isolation with a compound of the formula (XXI)

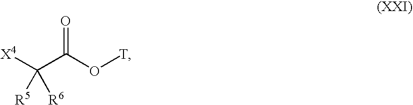

in which T, R⁵ and R⁶ are each as defined above
and
X⁴ represents a suitable leaving group, such as, for example, halogen, mesylate, tosylate or triflate,
   to produce compounds of the formula (XXII)

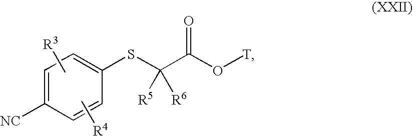

in which T, R³, R⁴, R⁵ and R⁶ are each as defined above,
then reducing with a suitable reducing agent, such as, preferably, borane or borane complexes (for example diethylaniline, dimethyl sulfide or tetrahydrofuran complexes) or else with sodium borohydride in combination with aluminum chloride to compounds of the formula (II-A)

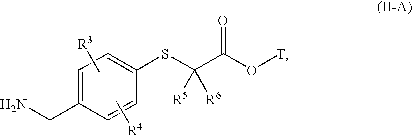

in which T, R³, R⁴, R⁵ and R⁶ are each as defined above, and subsequently, if appropriate, reacting these in the presence of a base with a compound of the formula (XXIII)

R^{2A}-X⁵ (XXIII),

in which
R^{2A} has the meaning of R² given above, but does not represent hydrogen,
   and
X⁵ represents a suitable leaving group, such as, for example, halogen, mesylate, tosylate or triflate.

Inert solvents for the process steps (II)+(III)→(IV), (IV)+(V)→(VI), (VI)+(VII)→(VIII), (X)+(XI)→(XII), (II)+(XVII)→(XVIII) and (II-A)+(XXIII)→(II) are, for example, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as ethyl acetate, acetone, dimethylformamide, dimethyl sulfoxide, N,N′-dimethylpropyleneurea (DMPU), N-methylpyrrolidone (NMP), pyridine, triethylamine or acetonitrile. It is also possible to use mixtures of the solvents mentioned. For the process steps (II)+(III)→(IV), (VI)+(VII)→(VIII), (X)+(XI)→(XII) and (II-A)+(XXIII)→(II), preference is in each case given to dimethylformamide, for the process step (IV)+(V)→(VI), preference is given to triethylamine, and for the process step (II)+(XVI)→(XVIII), preference is given to dioxane.

Inert solvents for the process step (II)+(IX)→(X) are, for example, alcohols, such as methanol, ethanol, n-propanol, isopropanol, n-butanol or tert-butanol, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as ethyl acetate, acetone, dimethylformamide, dimethyl sulfoxide, N,N′-dimethylpropyleneurea (DMPU), N-methylpyrrolidone (NMP), pyridine, triethylamine or acetonitrile. It is also possible to use mixtures of the solvents mentioned. Preference is given to dimethylformamide or isopropanol.

Suitable bases for the process steps (II)+(III)→(IV), (IV)+(V)→(VI), (VI)+(VII)→(VIII), (II)+(IX)→(X), (X)+(XI)→(XII), (X)+(XIII)→(XIV), (II)+(XVII)→(XVIII) and (II-A)+(XXIII)→(II) are the customary inorganic or organic bases. These preferably include alkali metal hydroxides, such as, for example, lithium hydroxide, sodium hydroxide or potassium hydroxide, alkali metal or alkaline earth metal carbonates, such as lithium carbonate, sodium carbonate, potassium carbonate, calcium carbonate or cesium carbonate, alkali metal alkoxides, such as sodium methoxide or potassium methoxide, sodium ethoxide or potassium ethoxide or potassium tert-butoxide, alkali metal hydrides, such as sodium hydride, amides, such as sodium amide, lithium bis(trimethylsilyl)amide or potassium bis(trimethylsilyl)amide or lithium diisopropylamide, or organic amines, such as triethylamine, N-methylmorpholine, N-methylpiperidine, N,N-diisopropylethylamine, pyridine, 1,5-diazabicyclo[4.3.0]non-5-ene (DBN), 1,4-diazabicyclo[2.2.2]-octane (DABCO®) or 1,8-diazabicyclo[5.4.0]undec-7-ene (DBU). For the process steps (IV)+(V)→(VI), (II)+(IX)→(X), (II)+(XVII)→(XVIII) and (II-A)+(XXIII)→(II), preference is given to triethylamine or N,N-diisopropylethylamine, for the process step (X)+(XI)→(XII), preference is given to sodium hydride or triethylamine, and for the process steps (II)+(III)(IV), (VI)+(VII)→(VIII) and (X)+(XIII)→(XIV), preference is given to potassium carbonate or cesium carbonate.

In these process steps, the base is in each case employed in an amount of from 1 to 5 mol, preferably in an amount of from 1 to 2.5 mol, based on 1 mol of the compound to be deprotonated. In process step (IV)+(V)→(VI), the base triethylamine can simultaneously be employed as solvent.

Inert solvents for the process step (X)+(XIII)→(XIV) are, for example, alcohols, such as methanol, ethanol, n-propanol, isopropanol, n-butanol or tert-butanol, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethyl sulfoxide, N,N′-dimethylpropyleneurea (DMPU), N-methylpyrrolidone (NMP), pyridine, acetonitrile or else water. It is also possible to use mixtures of the solvents mentioned. Preference is given to a mixture of glycol dimethyl ether, ethanol and water.

Inert solvents for the process step (X)+(XV)→(XVI) are, for example, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethyl sulfoxide, N,N′-dimethylpropyleneurea (DMPU), N-methylpyrrolidone (NMP), pyridine or acetonitrile. It is also possible to use mixtures of the solvents mentioned. Preference is given to tetrahydrofuran or dimethylformamide or a mixture of both.

The reactions are generally carried out in a temperature range of from 0° C. to +150° C. The process steps (II)+(III)→(IV), (IV)+(V)→(VI), (VI)+(VII)→(VIII) and (II-A)+(XXIII)→(II) are preferably carried out in a temperature range of from +10° C. to +50° C., the process step (II)+(IX)→(X) is preferably carried out in a range of from +20° C. to +80° C., the process steps (X)+(XI)→(XII), (II)+(XVII)→(XVIII) and (X)+(XIII)→(XIV) are preferably carried out in a range of from +80° C. to +150° C., and the process step (X)+(XV)→(XVI) is preferably carried out in a range of from +40° C. to +80° C.

The reactions can be carried out at atmospheric, elevated or reduced pressure (for example from 0.5 to 5 bar). In general, the reactions are carried out at atmospheric pressure.

Suitable palladium catalysts for the process step (IV)+(V)→(VI) (“Sonogashira coupling”) are, for example, palladium(II) chloride, bis(triphenylphosphine)palladium(II) chloride and tetrakis(triphenylphosphine)palladium(0) [cf., for example, T. E. Nielsen et al., J. Org. Chem. 67, 7309-7313 (2002)]. The reaction is preferably carried out in the presence of copper(I) iodide as cocatalyst [cf., for example, Chowdhuri et al., Tetrahedron 55, 7011 (1999)].

Suitable palladium catalysts for the process step (X)+(XIII)→(XIV) (“Suzuki coupling”) are, for example, palladium-on-carbon, palladium(II) acetate, tetrakis(triphenylphosphine)palladium(0), bis(acetonitrile)palladium(II) chloride and [1,1′-bis(diphenylphosphino)ferrocene]dichloropalladium(II) dichloromethane complex [cf., for example, J. Hassan et al., Chem. Rev. 102, 1359-1469 (2002)].

Suitable palladium catalysts for the process step (X)+(XV)→(XVI) (“Negishi coupling”) are, for example, bis(triphenylphosphine)palladium(II) chloride, tetrakis(triphenylphosphine)palladium(0), bis(dibenzylidenacetone)palladium(0) and [1,1′-bis(diphenylphosphino)ferrocene]dichloropalladium(II) dichloromethane complex [cf., for example, T. Shiota and T. Yamamori, J. Org. Chem. 64, 453-457 (1999)].

The hydrolysis of the carboxylic esters in the process steps (VIII), (XII), (XIV), (XVI) or (XVIII)→(I-B) is carried out by customary methods by treating the esters in inert solvents with bases, where the salts initially formed are converted by treatment with acid into the free carboxylic acids. In the case of the tert-butyl esters, the ester hydrolysis is preferably carried out using acids.

Suitable inert solvents for the hydrolysis of the carboxylic esters are water or the organic solvents customary for ester hydrolysis. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, acetonitrile, dichloromethane, dimethylformamide or dimethyl sulfoxide. It is also possible to use mixtures of the solvents mentioned. In the case of a basic ester hydrolysis, preference is given to using mixtures of water with dioxane, tetrahydrofuran, methanol and/or ethanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases for the ester hydrolysis are the customary inorganic bases. These preferably include alkali metal or alkaline earth metal hydroxides, such as, for example, sodium hydroxide, lithium hydroxide, potassium hydroxide or barium hydroxide, or alkali metal or alkaline earth metal carbonates, such as sodium carbonate, potassium carbonate or calcium carbonate. Particular preference is given to using sodium hydroxide or lithium hydroxide.

Suitable acids for the ester cleavage are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid or mixtures thereof, if appropriate with the addition of water. Preference is given to hydrogen chloride or trifluoroacetic acid in the case of the tert-butyl esters and to hydrochloric acid in the case of the methyl esters.

The ester hydrolysis is generally carried out in a temperature range of from −20° C. to +100° C., preferably from 0° C. to +50° C. The reactions can be carried out at atmospheric, elevated or reduced pressure (for example from 0.5 to 5 bar). In general, the reactions are carried out at atmospheric pressure.

The process step (I-B)→(I) is carried out according to methods known from the literature for the esterification or amidation (amide formation) of carboxylic acids.

Inert solvents for these process steps are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as ethyl acetate, pyridine, dimethyl sulfoxide, dimethylformamide, N,N′-dimethylpropyleneurea (DMPU), N-methylpyrrolidone (NMP), acetonitrile or acetone. It is also possible to use mixtures of the solvents mentioned. Preference is given to dichloromethane, tetrahydrofuran, dimethylformamide or mixtures of these solvents.

Suitable condensing agents for an esterification or amide formation in process step (I-B)→(I) are, for example, carbodiimides, such as N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC), or phosgene derivatives, such as N,N′-carbonyldiimidazole, or 1,2-oxazolium compounds, such as 2-ethyl-5-phenyl-1,2-oxazolium 3-sulfate or 2-tert-butyl-5-methylisoxazolium perchlorate, or acylamino compounds, such as 2-ethoxy-1-ethoxycarbonyl-1,2-dihydroquinoline, or isobutyl chloroformate, propanephosphonic anhydride, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries, such as 1-hydroxybenzotriazole (HOBt) or N-hydroxysuccininide (HOSu), and suitable bases are alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or N,N-diisopropylethylamine. Preference is given to using HATU or TCTU in combination with N,N-diisopropylethylamine.

The process step (I-B)→(I) is generally carried out in a temperature range of from −20° C. to +60° C., preferably from −10° C. to +40° C. The reaction can be carried out at atmospheric, elevated or reduced pressure (for example from 0.5 to 5 bar). In general, the reaction is carried out at atmospheric pressure.

The compounds of the formulae (III), (V), (VII), (IX), (XI), (XIII), (XV), (XVII), (XIX), (XXI) and (XXIII) are commercially available, known from the literature or can be prepared analogously to processes known from the literature.

The preparation of the compounds of the invention can be illustrated by the synthesis schemes below:

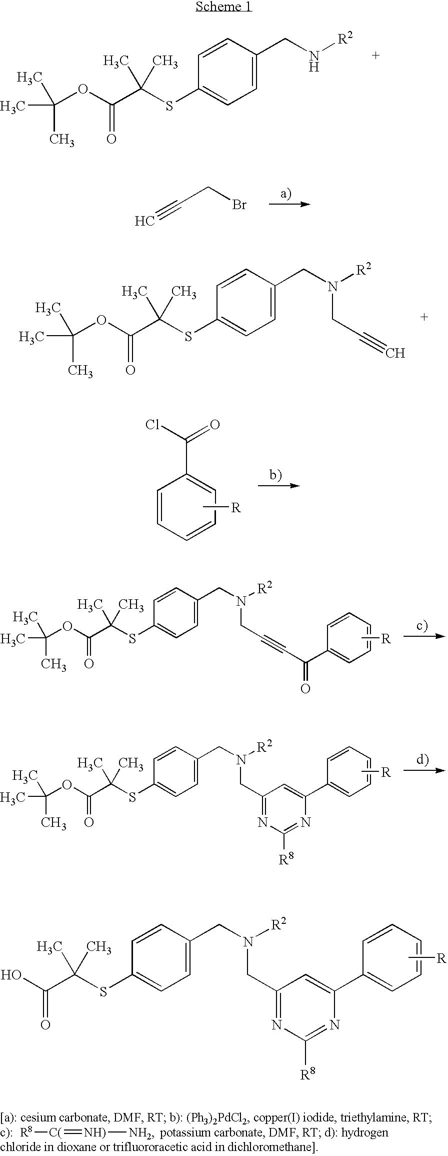

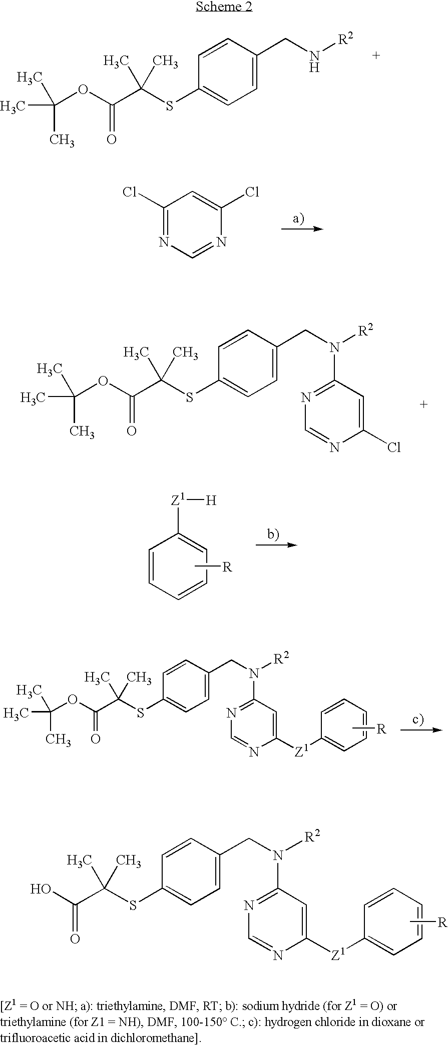

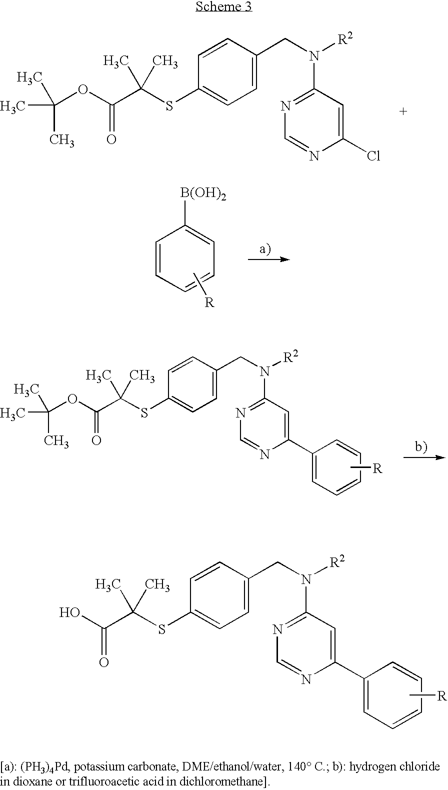

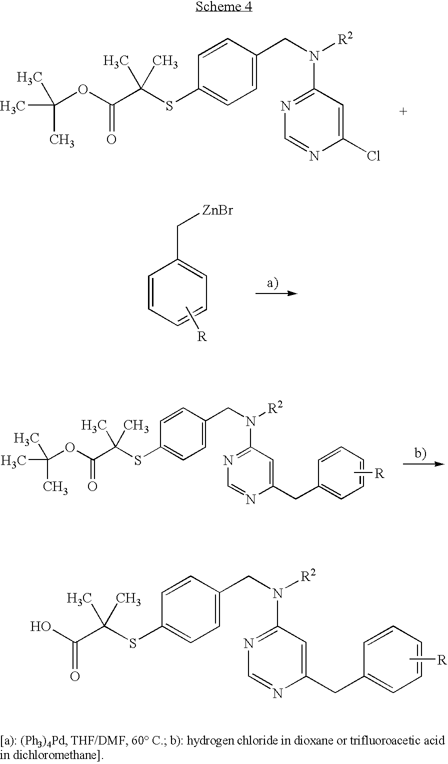

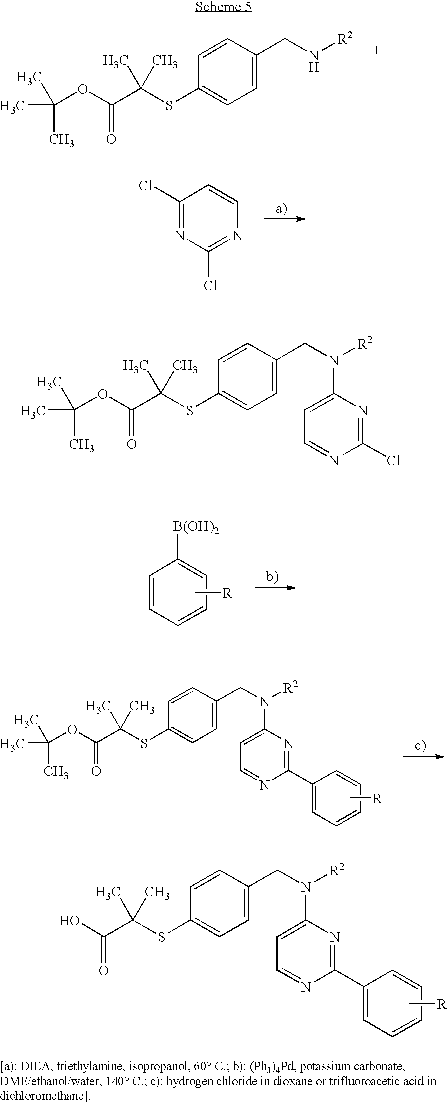

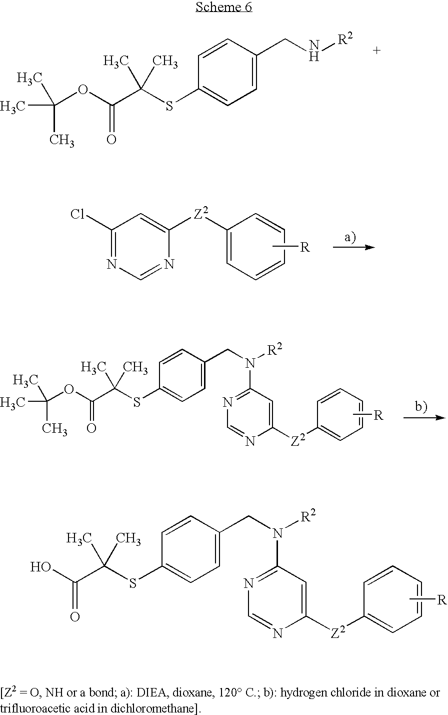

The compounds of the invention have useful pharmacological properties and can be used for the prevention and treatment of disorders in humans and animals.

The compounds of the invention are highly effective PPAR-alpha modulators and as such are suitable in particular for the primary and/or secondary prevention and treatment of cardiovascular disorders.

The compounds of the invention are particularly suitable for the treatment and prevention of coronary heart disease, for myocardial infarction prophylaxis and for the treatment of restenosis after coronary angioplasty or stenting. The compounds of the invention are also preferably suitable for treating stroke, CNS disorders, Alzheimer's disease, osteoporosis, arteriosclerosis, hypercholesterolaemia and for elevating pathologically low HDL levels and for lowering elevated triglyceride and LDL levels. In addition, they can be used for treating obesity, diabetes, metabolic syndrome (glucose intolerance, hyperinsulinemia, dyslipidemia and high blood pressure) and hepatic fibrosis.

In addition, the compounds of the invention can be used for the treatment of elevated concentrations of postprandial plasma triglycerides, of combined hyperlipidemias, insulindependent diabetes, non-insulin-dependent diabetes, hyperinsulinemia, insulin resistance and late sequelae of diabetes, such as retinopathy, nephropathy and neuropathy.

Further independent risk factors for cardiovascular disorders which can be treated by the compounds of the invention are high blood pressure, ischemia, myocardial infarction, angina pectoris, cardiac insufficiency, elevated levels of fibrinogen and of LDL of low density and also elevated concentrations of plasminogen activator inhibitor 1 (PAI-1).

Furthermore, the compounds of the invention can also be used for the treatment and/or prevention of micro- and macrovascular damage (vasculitis), reperfusion damage, arterial and venous thromboses, oedema, cancerous disorders (skin cancer, liposarcomas, carcinomas of the gastrointestinal tract, of the liver, of the pancreas, of the lung, of the kidney, of the urethra, of the prostate and of the genital tract), of neurodegenerative disorders (Parkinson's disease, dementia, epilepsy, depressions, multiple sclerosis), of inflammatory disorders, immune disorders (Crohn's disease, ulcerative colitis, lupus erythematodes, rheumatoid arthritis, asthma), renal disorders (glomerulonephritis), disorders of the thyroid gland, disorders of the pancreas (pancreatitis), skin disorders (psoriasis, acne, eczema, neurodermitis, dermatitis, keratitis, formation of scars, formation of warts, frostbites), viral diseases (HPV, HCMV, HIV, HAV, HBV, HCV), cachexia, gout, incontinence, for wound healing and angiogenesis, and also for improving performance.

The activity of the compounds of the invention can be examined, for example in vitro, by the transactivation assay described in the experimental section.

The in vivo activity of the compounds of the invention can be examined, for example, by the tests described in the experimental section.

The present invention furthermore provides the use of the compounds of the invention for the treatment and/or prophylaxis of disorders, in particular the disorders mentioned above.

The present invention also provides the use of the compounds of the invention for preparing a medicament for the treatment and/or prophylaxis of disorders, in particular the disorders mentioned above.

The present invention also provides a method for the treatment and/or prevention of disorders, in particular the disorders mentioned above, using an effective amount of at least one of the compounds of the invention.

The compounds of the invention can be used alone ot, if required, in combination with other active compounds. The present invention furthermore provides medicaments comprising at least one of the compounds of the invention and one or more further active compounds, in particular for the treatment and/or prophylaxis of the disorders mentioned above.

Suitable active compounds for combinations are, by way of example and by way of preference: substances which modulate lipid metabolism, such as PPAR-gamma and/or PPAR-delta agonists, CETP inhibitors, thyroid hormones and/or thyroid mimetics, inhibitors of HMG-CoA reductase, inhibitors of HMG-CoA reductase expression, squalene synthesis inhibitors, ACAT inhibitors, cholesterol absorption inhibitors, bile acid absorption inhibitors, MTP inhibitors, niacin receptor agonists, aldolase reductase inhibitors, and also lipase inhibitors; antidiabetics; antioxidants; hypotensive agents, such as calcium antagonists, angiotensin-II receptor antagonists, ACE inhibitors, alpha-receptor blockers, beta-receptor blockers; perfusion-enhancing and/or antithrombotic agents, such as platelet aggregation inhibitors, anticoagulants, profibrinolytic substances; anorectics, and also cytostatics. Further possible combinations include antiinflammatory agents, such as, for example, COX-2 inhibitors, and also NEP inhibitors, ECE inhibitors, vasopeptidase inhibitors, aldose reduction inhibitors and perfusion promoters.

If required, the compounds of the invention can furthermore be administered in combination with other active compounds, preferably from the group of the chemokine receptor antagonists, p38-kinase inhibitors, NPY agonists, orexin agonists, PAF-AH inhibitors, CCK-1 receptor antagonists, leptin receptor agonists, LTB₄-receptor antagonists, analgesics, antidepressants and other psychopharmaceuticals.

The present invention provides in particular combinations comprising at least one of the compounds of the invention and at least one lipid metabolism-modulating active compound, an antidiabetic, a hypotensive compound and/or an antithrombotic agent.

Preferably, the compounds of the invention can be combined with one or more
antidiabetics mentioned in the Rote Liste 2002/II, Chapter 12,
antithrombotic agents, by way of example and by way of preference from the group of the platelet aggregation inhibitors, the anticoagulants or the profibrinolytic substances,

hypotensives, by way of example and by way of preference from the group of the calcium antagonists, angiotensin-AII antagonists, ACE inhibitors, alpha-receptor blockers, betareceptor blockers and also the diuretics, and/or
lipid metabolism-modulating active compounds, by way of example and by way of preference from the group of the thyroid receptor agonists, cholesterol synthesis inhibitors, such as, by way of example and by way of preference, HMG-CoA reductase or squalene synthesis inhibitors, ACAT inhibitors, CETP inhibitors, MTP inhibitors, PPAR-gamma and/or PPARdelta agonists, cholesterol absorption inhibitors, lipase inhibitors, polymeric bile acid absorbers, bile acid reabsorption inhibitors and lipoprotein(a) antagonists.

Antidiabetics are preferably understood as meaning insulin and insulin derivatives, and also orally active hypoglycemic active compounds. Here, insulin and insulin derivatives include both insulins of animal, human or biotechnological origin, and also mixtures thereof.

Orally active hypoglycemic compounds include, by way of example and by way of preference, sulfonylurea, biguanides, meglitinide derivatives, oxadiazolidinones, thiazolidindiones, glucosidase inhibitors, glucagon antagonists, GLP-1 agonists, CCK-1 receptor agonists, leptin receptor agonists, insulin sensitizers, inhibitors of liver enzymes which are involved in the stimulation of gluconeogenesis and/or glycogenolysis, modulators of glucose uptake and potassium channel openers, such as, for example, those disclosed in WO 97/26265 and WO 99/03861.

In a preferred embodiment of the invention, the compounds of the invention are administered in combination with insulin.

In a preferred embodiment of the invention, the compounds of the invention are administered in combination with a glucosidase inhibitor, such as, by way of example and by way of preference, miglitol or acarbose.

In a preferred embodiment of the invention, the compounds of the invention are administered in combination with a sulfonylurea, such as, by way of example and by way of preference, tolbutamide, glibenclamide, glimepiride, glipizide or gliclazide.

In a preferred embodiment of the invention, the compounds of the invention are administered in combination with a biguanide, such as, by way of example and by way of preference, metformine.

In a preferred embodiment of the invention, the compounds of the invention are administered in combination with a meglitinide derivative, such as, by way of example and by way of preference, repaglinide or nateglinide.

In a preferred embodiment of the invention, the compounds of the invention are administered in combination with a PPAR-gamma agonist, for example from the class of the thiazolidinediones, such as, by way of example and by way of preference, pioglitazone or rosiglitazone.

Antithrombotic agents are preferably understood as meaning compounds from the group of the platelet aggregation inhibitors, the anticoagulants or the profibrinolytic substances.

In a preferred embodiment of the invention, the compounds of the invention are administered in combination with a platelet aggregation inhibitor, such as, by way of example and by way of preference, aspirin, clopidogrel, ticlopidine or dipyridamol.

In a preferred embodiment of the invention, the compounds of the invention are administered in combination with a thrombin inhibitor, such as, by way of example and by way of preference, ximelagatran, melagatran, bivalirudin or clexane.

In a preferred embodiment of the invention, the compounds of the invention are administered in combination with a GPIIb/IIIa antagonist, such as, by way of example and by way of preference, tirofiban or abciximab.

In a preferred embodiment of the invention, the compounds of the invention are administered in combination with a factor Xa inhibitor, such as, by way of example and by way of preference, BAY 59-7939, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low-molecular-weight (LMW) heparin derivative.

In a preferred embodiment of the invention, the compounds of the invention are administered in combination with a vitamin K antagonist, such as, by way of example and by way of preference, coumarine.

Hypotensives are preferably understood as meaning compounds from the group of the calcium antagonists, angiotensin All antagonists, ACE inhibitors, alpha-receptor blockers, beta-receptor blockers, phosphodiesterase inhibitors, sGC stimulators/sGC activators, enhancers of cGMP concentrations, aldosterone antagonists/mineralocorticoid receptor antagonists and also the diuretics.

In a preferred embodiment of the invention, the compounds of the invention are administered in combination with a calcium antagonist, such as, by way of example and by way of preference, nifedipine, amlodipine, verapamil or diltiazem.

In a preferred embodiment of the invention, the compounds of the invention are administered in combination with an alpha-1-receptor blocker, such as, by way of example and by way of preference, prazosine.

In a preferred embodiment of the invention, the compounds of the invention are administered in combination with a beta-receptor blocker, such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with antisympathotonics, such as reserpin, with potassium channel agonists, such as minoxidil, diazoxide, dihydralazine or hydralazine, or with nitric oxide-releasing substances, such as, by way of example and by way of preference, glycerol nitrate or nitroprusside sodium.

In a preferred embodiment of the invention, the compounds of the invention are administered in combination with an angiotensin-AII antagonist, such as, by way of example and by way of preference, losartan, candesartan, valsartan, telmisartan or embursatan.

In a preferred embodiment of the invention, the compounds of the invention are administered in combination with an ACE inhibitor, such as, by way of example and by way of preference, enalapril, captopril, lisinopril, ramipril, delapril, fosinopril, quinopril, perindopril or trandopril.

In a preferred embodiment of the invention, the compounds of the invention are administered in combination with a diuretic, such as, by way of example and by way of preference, furosemide.

Lipid metabolism-modulating agents are to be understood as meaning, by way of example and by way of preference, compounds from the group of the CETP inhibitors, thyroid receptor agonists, cholesterol synthesis inhibitors, such as HMG-CoA reductase or squalene synthesis inhibitors, ACAT inhibitors, MTP inhibitors, PPAR-gamma and/or PPAR-delta agonists, cholesterol absorption inhibitors, polymeric bile acid adsorbers, bile acid reabsorption inhibitors, aldolase reductase inhibitors, lipase inhibitors, lipoprotein(a) antagonists, RXR modulators, FXR modulators, LXR modulators, ATP-citrate lyase inhibitors, leptin receptor agonists, cannabinoid receptor-1 antagonists, bombesin receptor agonists, niacin receptor agonists, histamine receptor agonists, free-radical quenchers and LDL receptor inducers.

In a preferred embodiment of the invention, the compounds of the invention are administered in combination with a CETP inhibitor, such as, by way of example and by way of preference, torcetrapib (CP-529 414), JJT-705 or CETP-vaccine (Avant).

In a preferred embodiment of the invention, the compounds of the invention are administered in combination with a thyroid receptor agonist, such as, by way of example and by way of preference, D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of the statins, such as, by way of example and by way of preference, lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor, such as, by way of example and by way of preference, BMS-188494 or TAK-475.

In a preferred embodiment of the invention, the compounds of the invention are administered in combination with an ACAT inhibitor, such as, by way of example and by way of preference, avasimibe, melinamide, pactimibe, eflucimibe or SMP-797.

In a preferred embodiment of the invention, the compounds of the invention are administered in combination with an MTP inhibitor, such as, by way of example and by way of preference, implitapide, BMS-201038, R-103757 or JTT-130.

In a preferred embodiment of the invention, the compounds of the invention are administered in combination with a PPAR-gamma agonist, such as, by way of example and by way of preference, pioglitazone or rosiglitazone.

In a preferred embodiment of the invention, the compounds of the invention are administered in combination with a PPAR-delta agonist, such as, by way of example and by way of preference, GW 501516 or BAY 68-5042.

In a preferred embodiment of the invention, the compounds of the invention are administered in combination with a cholesterol absorption inhibitor, such as, by way of example and by way of preference, ezetimibe, tiqueside or pamaqueside.

In a preferred embodiment of the invention, the compounds of the invention are administered in combination with a lipase inhibitor, such as, by way of example and by way of preference, orlistat.

In a preferred embodiment of the invention, the compounds of the invention are administered in combination with a polymeric bile acid adsorber, such as, by way of example and by way of preference, cholestyramine, colestipol, colesolvam, cholestagel or colestimid.

In a preferred embodiment of the invention, the compounds of the invention are administered in combination with a bile acid reabsorption inhibitor, such as, by way of example and by way of preference, ASBT(=IBAT) inhibitors, such as, for example, AZD-7806, S-8921, AK-105, BARI-1741, SC435 or SC-635.

In a preferred embodiment of the invention, the compounds of the invention are administered in combination with a lipoprotein(a) antagonist, such as, by way of example and by way of preference, gemcabene calcium (CI-1027) or nicotinic acid.

In a preferred embodiment of the invention, the compounds of the invention are administered in combination with a cannabinoid receptor-1 antagonist, such as, by way of example and by way of preference, rimonabant or SR-147778.

In a preferred embodiment of the invention, the compounds of the invention are administered in combination with a niacin receptor agonist, such as, by way of example and by way of preference, niacin, acipimox, acifran or radecol.

In a preferred embodiment of the invention, the compounds of the invention are administered in combination with an antioxidant/free-radical quencher, such as, by way of example and by way of preference, probucol, AGI-1067, BO-653 or AEOL-10150.

The present invention furthermore provides medicaments comprising at least one compound of the invention, usually together with one or more inert non-toxic pharmaceutically suitable auxiliaries, and their use for the purposes mentioned above.

The compounds of the invention can act systemically and/or locally. For this purpose, they can be administered in a suitable manner, such as, for example, orally, parenterally, pulmonally, nasally, sublingually, lingually, buccally, rectally, dermally, transdermally, conjunctivally, otically or as an implant or stent.

For these administration routes, the compounds of the invention can be administered in suitable administration forms.

Suitable for oral administration are administration forms which work in accordance with the prior art and release the compounds of the invention rapidly and/or in modified form and which comprise the compounds of the invention in crystalline and/or amorphicized and/or dissolved form, such as, for example, tablets (uncoated or coated tablets, for example with enteric coats or coats which dissolve in a delayed manner or are insoluble and which control the release of the compounds of the invention), films/wafers or tablets which dissolve rapidly in the oral cavity, films/lyophilizates, capsules (for example hard or soft gelatin capsules), sugar-coated tablets, granules, pellets, powders, emulsions, suspensions, aerosols or solutions.

Parenteral administration may take place by circumventing a bioabsorption step (for example intravenously, intraarterially, intracardially, intraspinally or intralumbarly), or with bioabsorption (for example intramuscularly, subcutaneously, intracutaneously, percutaneously or intraperitoneally). Administration forms suitable for parenteral administration are inter alia preparations for injection or infusion in the form of solutions, suspensions, emulsions, lyophilizates or sterile powders.

Suitable for other administration routes are, for example, medicaments suitable for inhalation (inter alia powder inhalers, nebulizers), nose drops, solutions or sprays, tablets to be administered lingually, sublingually or buccally, films/wafers or capsules, suppositories, preparations to be administered to ears or eyes, vaginal capsules, aqueous suspensions (lotions, shaking mixtures), lipophilic suspensions, ointments, creams, transdermal therapeutic systems (for example plasters), milk, pastes, foams, powders for pouring, implants or stents.

Preference is given to oral or parenteral administration, in particular to oral administration.

The compounds of the invention can be converted into the administration forms mentioneof theoryis can be carried out in a manner known per se by mixing with inert non-toxic pharmaceutically suitable auxiliaries. These auxiliaries include inter alia carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides), and flavor and/or odor corrigents.

In general, it has been found to be advantageous in the case of parenteral administration to administer amounts of about 0.001 to 1 mg/kg, preferably about 0.01 to 0.5 mg/kg, of body weight to obtain effective results. In the case of oral administration, the dosage is from about 0.01 to 100 mg/kg, preferably from about 0.01 to 20 mg/kg and very particularly preferably from 0.1 to 10 mg/kg of body weight.

In spite of this, it may be necessary to deviate from the amounts mentioned, namely depending on body weight, administration route, individual response to the active compound, the type of preparation and the time or the interval at which administration takes place. Thus, in some cases it may be sufficient to administer less than the abovementioned minimum amount, whereas in other cases the upper limit mentioned has to be exceeded. In the case of the administration of relatively large amounts, it may be expedient to divide these into a plurality of individual doses which are administered over the course of the day.

The working examples below illustrate the invention. The invention is not limited to the examples.

The percentages in the tests and examples below are, unless indicated otherwise, percentages by weight; parts are parts by weight. Solvent ratios, dilution ratios and concentrations of liquid/liquid solutions are in each case based on volume.

### A. EXAMPLES

### Abbreviations

abs. absolute
br. s broad singlet (in NMR)
d day(s)
TLC thin-layer chromatography
DCI direct chemical ionization (in ms)
DIEA N,N diisopropylethylamine
DME 1,2-dimethoxyethane
DMF dimethylformamide
DMSO dimethyl sulfoxide
eq. equivalent(s)
ESI electrospray ionization (in MS)
EtOAc ethyl acetate
GC gas chromatography
h hour(s)
HPLC high-pressure, high-performance liquid chromatography
LC/MS liquid chromatography-coupled mass spectroscopy
min minute(s)
MS mass spectroscopy
MTBE methyl tert-butyl ether
NMP N-methylpyrrolidinone
NMR nuclear magnetic resonance spectroscopy
Ph phenyl
RT room temperature
Rₜ retention time (in HPLC)
TBAI tetra-n-butylammonium iodide
TFA trifluoroacetic acid
THF tetrahydrofuran
UV ultraviolet spectroscopy
* unexpected multiplicity of signals, caused, for example, by random isochronicity (in NMR)

### LC/MS and HPLC Methods:

### Method 1 (LC/MS):

Instrument MS: Micromass ZQ; instrument HPLC: Waters Alliance 2795; column: Phenomenex Synergi 2μ Hydro-RP Mercury 20 mm×4 mm; mobile phase A: 11 of water+0.5 ml of 50% strength formic acid, mobile phase B: 11 of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 2 (LC/MS):

Instrument: Micromass Quattro LCZ with HPLC Agilent series 1100; column: Phenomenex Synergi 2μ Hydro-RP Mercury 20 mm×4 mm; mobile phase A: 11 of water+0.5 ml of 50% strength formic acid, mobile phase B: 11 of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 3 (LC/MS):

Instrument MS: Micromass ZQ; instrument HPLC: HP 1100 series; UV DAD; column: Phenomenex Synergi 2μ Hydro-RP Mercury 20 mm×4 mm; mobile phase A: 11 of water+0.5 ml of 50% strength formic acid, mobile phase B: 11 of acetonitrile+0.5 ml of 50% strength formic acid; gradient: 0.0 min 90% A→2.5 min 30% A→3.0 min 5% A→4.5 min 5% A; flow rate: 0.0 min 1 ml/min ˜2.5 min/3.0 min/4.5 min 2 ml/min; oven: 50° C.; UV detection: 210 nm.

### Method 4 (LC/MS):

Instrument MS: Micromass TOF (LCT); instrument HPLC: 2-column set up, Waters 2690; column: YMC-ODS-AQ, 50 mm×4.6 mm, 3.0 μm; mobile phase A: water+0.1% formic acid, mobile phase B: acetonitrile+0.1% formic acid; gradient: 0.0 min 100% A→0.2 min 95% A→1.8 min 25% A→1.9 min 10% A→2.0 min 5% A→3.2 min 5% A; oven: 40° C.; flow rate: 3.0 ml/min; UV detection: 210 n.

### Method 5 (LC/MS):

Instrument: Micromass Platform LCZ with HPLC Agilent series 1100; column: Thermo HyPURITY Aquastar 3μ50 mm×2.1 mm; mobile phase A: 111 of water+0.5 ml of 50% strength formic acid, mobile phase B: 111 of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 6 (LC/MS):

Instrument MS: Micromass ZQ; instrument HPLC: Waters Alliance 2795; column: Merck Chromolith SpeedROD RP-18e 50 mm×4.6 mm; mobile phase A: water+500 μl of 50% strength formic acid/l; mobile phase B: acetonitrile+500 μl of 50% strength formic acid/l; gradient: 0.0 min 10% B→3.0 min 95% B→4.0 min 95% B; oven: 35° C.; flow rate: 0.0 min 1.0 ml/min→3.0 min 3.0 ml/min ˜4.0 min 3.0 ml/min; UV detection: 210 nm.

### Method 7 (LC/MS):

Instrument: Micromass Platform LCZ with HPLC Agilent series 1100; column: Thermo Hypersil GOLD 3μ 20 mm×4 mm; mobile phase A: 111 of water+0.5 ml of 50% strength formic acid, mobile phase B: 111 of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Starting Materials and Intermediates:

### Example 1A

### tert-Butyl 2-[(4-{[(2-furylmethyl)(prop-2-yn-1-yl)amino]methyl}phenyl)thio]-2-methylpropanoate

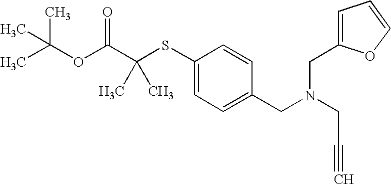

10.0 g of tert-butyl 2-[(4-{[(2-furylmethyl)amino]methyl}phenyl)thio]-2-methylpropanoate hydrochloride (25.13 mmol) [prepared according to WO 02/28821, Example 1′-3] are suspended in 100 ml of DMF. 16.37 g of cesium carbonate (50.26 mmol) and 2.99 g of 3-bromo-1-propyne (25.13 mmol) are added, and the mixture is then stirred at RT overnight. After the reaction has ended (monitored by TLC), 250 ml of water are added and the mixture is extracted with dichloromethane. The organic phases are dried, the solvent is distilled off under reduced pressure and the residue is then purified by column chromatography (silica gel, mobile phase: cyclohexane/ethyl acetate 10:1). This gives 4.67 g (43% of theory) of the title compound.

LC/MS (method 1): Rₜ, =2.99 min; MS (ESIpos): m/z=400 [M+H]⁺.

¹H-NMR (400 MHz, DMSO-d₆): δ [ppm]=1.33 (s, 9H), 1.37 (s, 6H), 3.20 (m, 2H), 3.23 (t, 1H), 3.64 (d, 4H), 6.32 (d, 1H), 6.40 (dd, 1H), 7.33 (d, 2H), 7.43 (d, 2H), 7.61 (m, 1H).

### Example 2A

### tert-Butyl 2-{[4-({(2-furylmethyl)[4-(4-methylphenyl)-4-oxobut-2-yn-1-yl]amino}methyl)phenyl]-thio}-2-methylpropanoate

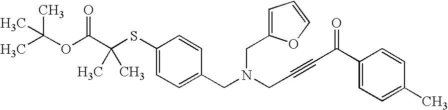

In a flask which was dried by heating, 1.41 mg of bis-triphenylphosphinepalladium chloride (0.002 mmol) and 1.91 mg of copper(I) iodide (0.01 mmol) are initially charged in 5 ml triethylamine under a stream of argon. After addition of 101 mg of p-toloyl chloride (0.65 mmol) and 200 mg of the compound from Example 1A (0.50 mmol) the mixture is stirred at RT overnight. After the reaction has ended (monitored by TLC), water is added and the mixture is extracted twice with ethyl acetate. The organic phases are dried, the solvent is distilled off under reduced pressure and the residue is then purified by preparative HPLC (mobile phase: acetonitrile/water with 0.1% formic acid, gradient 20:80→95:5). This gives 147 mg (57% of theory) of the title compound.

LC/MS (method 2): Rₜ=3.46 min; MS (ESIpos): m/z=518 [M+H]⁺.

¹H-NMR (400 MHz, DMSO-d₆): δ [ppm]=1.33 (s, 9H), 1.37 (s, 6H), 2.42 (s, 3H), 3.63 (s, 2H), 3.76 (s, 2H), 3.77 (s, 2H), 6.38 (d, 1H), 6.41 (m, 1H), 7.38 (d, 2H), 7.44 (t, 4H), 7.61 (d, 1H), 7.99 (d, 2H).

### Example 3A

### tert-Butyl 2-({4-[((2-furylmethyl) {[6-(4-methylphenyl)pyrimidin-4-yl]methyl}amino)methyl]-phenyl}thio)-2-methylpropanoate

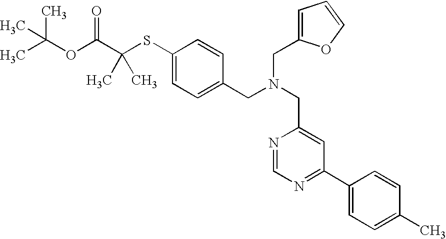

148 mg of the compound from Example 2A (0.29 mmol) and 28 mg of formamidine hydrochloride (0.34 mmol) are taken up in 5 ml DMF. After addition of 99 mg of potassium carbonate (0.71 mmol), the mixture is stirred at RT for three days. Water is then added, and the mixture is extracted twice with diethyl ether. The combined organic phases are dried, the solvent is distilled off under reduced pressure and the residue is then purified by preparative HPLC (mobile phase: acetonitrile/water with 0.1% formic acid, gradient 20:80→95:5). This gives 67 mg (43% of theory) of the title compound.

LC/MS (method 1): Rₜ=3.29 min; MS (ESIpos): m/z=544 [M+H]⁺.

¹H-NMR (400 MHz, DMSO-d₆): δ [ppm]=1.26 (s, 9H), 1.34 (s, 6H), 2.40 (s, 3H), 3.74 (s; 4H), 3.76 (s, 2H), 6.34 (d, 1H), 6.38 (dd, 1H), 7.38 (d, 2H), 7.43 (s, 4H), 7.60 (d, 1H), 8.01-8.07 (m, 3H), 9.07 (d, 1H).

### Example 4A

### tert-Butyl 2-({4-[((2-furylmethyl) {4-oxo-4-[3-(trifluoromethyl)phenyl]but-2-yn-1-yl}amino)methyl]phenyl}thio)-2-methylpropanoate

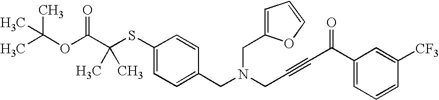

Analogously to the preparation of Example 2A, 250 mg of the compound from Example 1A (0.63 mmol) are reacted with 170 mg of 3-trifluoromethylbenzoyl chloride (0.81 mmol). Purification by preparative HPLC (mobile phase: acetonitrile/water with 0.1% formic acid, gradient 20:80→95:5) gives 129 mg (36% of theory) of the title compound.

LC/MS (method 3): Rₜ=3.48 min; MS (ESIpos): m/z=572 [M+H]⁺.

¹H-NMR (400 MHz, DMSO-d₆): δ [ppm]=1.32 (s, 9H), 1.36 (s, 6H), 3.68 (s, 2H), 3.78 (s, 2H), 3.80 (s, 2H), 6.39 (s, 2H), 7.38 (d, 2H), 7.44 (d, 2H), 7.59 (s, 1H), 7.89 (t, 1H), 8.14 (m, 1H), 8.32-8.38 (m, 2H).

### Example 5A

### tert-Butyl 2-({4-[((2-furylmethyl) {[6-(3-(trifluoromethyl)pyrimidinyl]methyl}amino)methyl]-phenyl}thio)-2-methylpropanoate

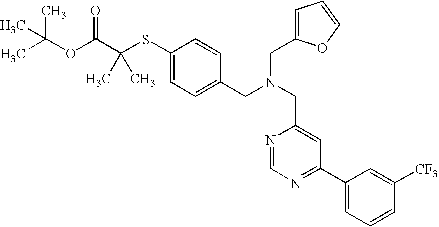

129 mg of the compound from Example 4A (0.23 mmol)—are dissolved in 3 ml of DMF. After addition of 28 mg of formamidine hydrochloride (0.34 mmol) and 0.14 ml of DIEA (0.79 mmol), the mixture is stirred at RT overnight. The reaction remains incomplete (monitored by TLC). The mixture is then heated at 50° C. for 1 h. The solvent is then distilled off under reduced pressure and the residue is purified by preparative HPLC (mobile phase: acetonitrile/water with 0.1% formic acid, gradient 20:80 4 95:5). This gives 65 mg (48% of theory) of the title compound.

LC/MS (method 1): Rₜ=3.31 min; MS (ESIpos): m/z=598 [M+H]⁺.

¹H-NMR (300 MHz, DMSO-d₆): δ [ppm]=1.26 (s, 9H), 1.32 (s, 6H), 3.76 (s, 4H), 3.82 (s, 2H), 6.35 (d, 1H), 6.38 (m, 1H), 7.39 (d, 2H), 7.44 (s, 2H), 7.58 (m, 1H), 7.84 (m, 1H), 7.96 (m, 1H), 8.17 (s, 1H), 8.45 (m, 2H), 9.16 (d, 1H).

### Example 6A

### tert-Butyl 2-[(4-{[(6-chloropyrimidin-4-yl)(2-furylmethyl)amino]methyl}phenyl)thio]-2-methylpropanoate

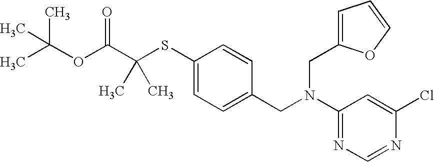

4.0 g of tert-butyl 2-[(4-{[(2-furylmethyl)amino]methyl}phenyl)thio]-2-methylpropanoate hydrochloride (10.05 mmol) [prepared according to WO 02/28821, Example II-3] are suspended in 20 ml of DMF. After addition of 1.57 g of 4,6-dichloropyrimidine (10.55 mmol) and 2.1 ml of triethylamine (15.08 mmol), the mixture is stirred at RT overnight. Water is added and the mixture is extracted twice with ethyl acetate. The combined organic phases are washed with water and dried with sodium sulfate, and the solvent is distilled off under reduced pressure. The residue is purified by flash chromatography (silica gel, mobile phase: cyclohexane/ethyl acetate 6:1). This gives 3.41 g (69% of theory) of the title compound.

LC/MS (method 3): Rₜ=3.25 min; MS (ESIpos): m/z=474 [M+H]⁺.

¹H-NMR (300 MHz, DMSO-d₆): δ [ppm]=1.32 (s, 9H), 1.35 (s, 6H), 4.84 (br. s, 4H), 6.35-6.40 (m, 2H), 6.76-7.15 (br. s, 1H), 7.18 (d, 2H), 7.39 (s, 2H), 7.59 (s, 1H), 8.39 (s, 1H).

### Example 7A

### tert-Butyl 2-[(4-{[(6-chloropyrimidin-4-yl)amino]methyl}phenyl)thio]-2-methylpropanoate

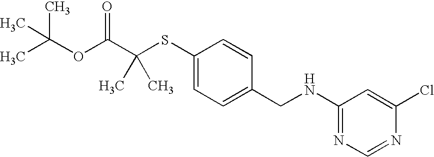

5.0 g of tert-butyl 2-{[4-(aminomethyl)phenyl]thio}-2-methylpropanoate hydrochloride (Example 34A, 15.73 mmol), 2.46 g of 4,6-dichloropyrimidine (16.52 mmol) and 2.19 ml of triethylamine (15.73 mmol) are initially charged in 30 ml of DMF and reacted at 50° C. overnight. Water is added, and the mixture is extracted twice with ethyl acetate. The combined organic phases are washed with water and dried with sodium sulfate, and the solvent is distilled off under reduced pressure. The residue is purified by flash chromatography (silica gel, mobile phase: cyclohexane/ethyl acetate 5:1). This gives 2.60 g (42% of theory) of the title compound.

LC/MS (method 2): Rₜ=2.87 nm; MS (ESIpos): m/z=394 [M+H]⁺.

¹H-NMR (400 MHz, DMSO-d₆): δ [ppm]=1.32 (br. s, 9H), 1.35 (s, 6H), 4.58 (br. s, 2H), 6.60 (s, 1H), 7.31 (d, 2H), 7.42 (d, 2H), 8.26 (s*, 2H).

### Example 8A

### tert-Butyl 2-[(4-{[(2-methoxyethyl)amino]methyl}phenyl)thio]-2-methylpropanoate

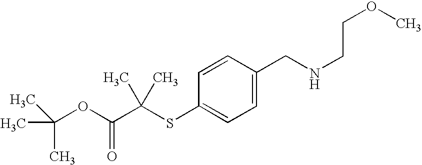

5.0 g of tert-butyl 2-{[4-(aminomethyl)phenyl]thio}-2-methylpropanoate hydrochloride (Example 34A, 15.73 mmol) are initially charged in 15 ml of DMF, and 1.97 g of 2-bromoethylmethylether (14.16 mmol) and 5.48 ml of triethylamine (39.32 mmol) are added at RT. The mixture is stirred at RT overnight and then concentrated using a rotary evaporator. Water is added to the residue, and the mixture is extracted twice with ethyl acetate. The organic phases are dried with sodium sulfate and the solvent is distilled off under reduced pressure. Work-up is by flash chromatography on silica gel (mobile phase: dichloromethane/isopropanol 5:1). This gives 2.56 g (48% of theory) of the title compound.

LC/MS (method 1): Rₜ 1.49 min; MS (ESIpos): m/z=340 [M+H]⁺.

¹H-NMR (400 MHz, DMSO-d₆): δ [ppm]=1.38 (s*, 15H), 3.09 (t, 2H), 3.30 (s, 3H), 3.58 (t, 2H), 4.18 (s, 2H), 7.51 (s*, 4H), 8.92 (br. s, 1H).

### Example 9A

### tert-Butyl 2-[(4-{[(6-chloropyrimidin-4-yl)(2-methoxyethyl)amino]methyl}phenyl)thio]-2-methylpropanoate

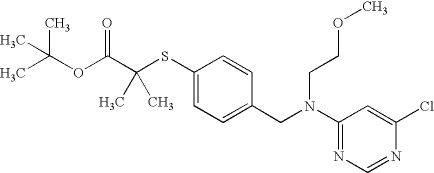

2.10 g of the compound from Example 8A (6.19 mmol), 0.97 g of 4,6-dichloropyrimidine (6.49 mmol) and 1.29 ml of triethylamine (9.28 mmol) are initialed charged in 20 ml of DMF and reacted at RT overnight. Water is added, and the mixture is extracted twice with ethyl acetate. The combined organic phases are washed with water and dried with sodium sulfate, and the solvent is distilled off under reduced pressure. The residue is purified by flash chromatography (silica gel, mobile phase: cyclohexane/ethyl acetate 5:1). This gives 1.53 g (55% of theory) of the title compound.

LC/MS (method 3): R_{t=3.14} min; MS (ESIpos): m/z=452 [M+H]⁺.

¹H-NMR (400 MHz, DMSO-d₆): δ [ppm]=1.31 (br. s, 9H), 1.35 (s, 6H), 3.22 (s, 3H), 3.50 (t, 2H), 3.68 (br. s*, 2H), 4.86 (br. s, 2H), 6.83 (br. s, 1H), 7.21 (d, 2H), 7.42 (d, 2H), 8.34 (s, 1H).

### Example 10

### tert-Butyl 2-{[4-({(2-furylmethyl)[6-(3-methylbenzyl)pyrimidin-4-yl]amino}methyl)phenyl]thio}-2-methylpropanoate

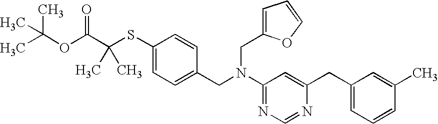

a) Preparation of the 3-methylbenzylzinc bromide:

In a flask which was dried by heating and under argon protective gas, 1.634 g of zinc dust (25 mmol) and 190 mg of 1,2-dibromoethane are stirred in 5 ml of abs. DMF at 70° C. for 10 min. The mixture is cooled to RT, 0.1 ml of chlorotrimethylsilane (0.80 mmol) is added and the mixture is stirred at RT for 30 min. 4.07 g of 3-methylbenzyl bromide (22 mmol) as a solution in 20 ml of DMF are then added dropwise over a period of 2 h. If required the zinc insertion is initiated by heating to about 60° C. The mixture is then stirred at RT for 2 h. This gives an about 0.5 molar solution which is directly reacted further.

### b) Practice of the Coupling Reaction:

Under a dynamic protective gas atmosphere, 200 mg of the compound from Example 6A (0.42 mmol) and 24 mg of tetrakis(triphenylphosphine)palladium(0) (0.021 mmol) are dissolved in 5 ml of abs. THF. 1.69 ml of the 3-methylbenzylzinc bromide solution described above (0.84 mmol) are then added, and the reaction mixture is reacted at 60° C. for 2 h. The mixture is cooled to RT, poured into 20 ml of saturated ammonium chloride solution and extracted with ethyl acetate (three times with in each case 20 ml). The combined organic phases are dried with sodium sulfate, the solvent is distilled off under reduced pressure and the residue is then purified by preparative HPLC (mobile phase: acetonitrile/water with 0.1% formic acid, gradient 20:80-95:5). This gives 96 mg (42% of theory) of the title compound.

LC/MS (method 1): Rₜ=2.67 min; MS (ESIpos): n/z=544 [M+H]⁺.

¹H-NMR (300 MHz, DMSO-d₆): δ [ppm]=1.32 (s, 9H), 1.34 (s, 6H), 2.24 (s, 3H), 3.77 (s, 2H), 4.77 (br. s, 4H), 6.28 (d, 1H), 6.36 (dd, 1H), 6.68 (br. s, 1H), 6.97-7.04 (m, 3H), 7.11-7.19 (m, 3H), 7.37 (d, 2H), 7.54 (d, 1H), 8.42 (s, 1H).

### Example 11A

### tert-Butyl 2-{[4-({(2-furylmethyl)[6-(4-methylbenzyl)pyrimidin-4-yl]amino}methyl)phenyl]thio}-2-methylpropanoate

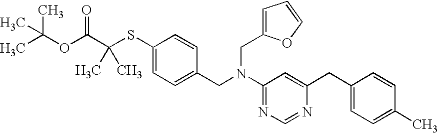

a) Preparation of the 4-methylbenzylzinc bromide:

In a flask which was dried by heating and under argon protective gas, 1.634 g of zinc dust (25 mmol) and 190 mg of 1,2-dibromoethane are stirred in 5 ml of abs. DMF at 70° C. for 10 min. The mixture is cooled to RT, 0.1 ml of chlorotrimethylsilane (0.80 mmol) is added and the mixture is stirred at RT for 30 min. 4.07 g of 4-methylbenzyl bromide (22 mmol) as a solution in 20 ml of DMF are then added dropwise over a period of 2 h. If required the zinc insertion is initiated by heating to about 60° C. The mixture is then stirred at RT for 2 h. This gives an about 0.5 molar solution which is directly reacted further.

### b) Practice of the Coupling Reaction:

Under a dynamic protective gas atmosphere, 200 mg of the compound from Example 6A (0.42 mmol) and 24 mg of tetrakis(triphenylphosphine)palladium(0) (0.021 mmol) are dissolved in 5 ml of abs. THF. 1.69 ml of the 4-methylbenzylzinc bromide solution described above (0.84 mmol) are then added, and the reaction mixture is reacted at 60° C. for 2 h. The mixture is cooled to RT, poured into 20 ml of saturated ammonium chloride solution and extracted with ethyl acetate (three times with in each case 20 ml). The combined organic phases are dried with sodium sulfate, the solvent is distilled off under reduced pressure and the residue is then purified by preparative HPLC (mobile phase: acetonitrile/water with 0.1% formic acid, gradient 20:80→95:5). This gives 164 mg (71% of theory) of the title compound.

LC/MS (method 3): Rₜ=2.82 min; MS (ESIpos): m/z=544 [M+H]⁺.

¹H-NMR (400 MHz, DMSO-d₆): δ [ppm]=1.32 (s, 9H), 1.35 (s, 6H), 2.25 (s, 3H), 3.76 (s, 2H), 4.77 (br. s, 4H), 6.28 (d, 1H), 6.36 (dd, 1H), 6.64 (br. s, 1H), 7.12-7.19 (m, 6H), 7.36 (d, 2H), 7.54 (d, 1H), 8.41 (s, 1H).

### Example 12A

### tert-Butyl 2-({4-[((2-methoxyethyl) {6-[3-(trifluoromethyl)phenyl]pyrimidin-4-yl}amino)methyl]-phenyl}thio)-2-methylpropanoate

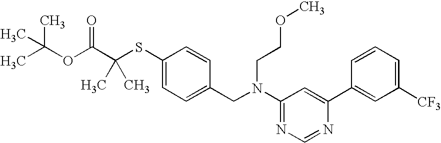

150 mg of the compound from Example 9A (0.33 mmol), 88 mg of 3-trifluoromethylphenylboronic acid (0.46 mmol), 92 mg of potassium carbonate (0.66 mmol) and 15 mg of tetrakis(triphenylphosphine)palladium(0) (0.01 mmol) are dissolved in 5 ml of 1,2-dimethoxyethane/ethanol (4:1), and 1.7 ml of water are added. In a pressure vessel, the mixture is then heated in a microwave to 140° C. for 30 min. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 101 mg (54% of theory) of the title compound.

LC/MS (method 2): Rₜ=3.35 min; MS (ESIpos): m/z=562 [M+H]⁺.

¹H-NMR (300 MHz, DMSO-d₆): δ [ppm]=1.27 (br. s, 9H), 1.34 (s, 6H), 3.24 (s, 3H), 3.56 (t, 2H), 3.84 (br. s*, 2H), 4.97 (s, 2H), 7.12-7.43 (br. s, 1H), 7.26 (d, 2H), 7.42 (d, 2H), 7.73 (t, 1H), 7.85 (d, 1H), 8.39 (br. s, 2H), 8.61 (s, 1H).

### Example 13A

### tert-Butyl 2-[(4-{[[6-(3-chlorophenyl)pyrimidinyl](2-methoxyethyl)amino]methyl}phenyl)thio]-2-methylpropanoate

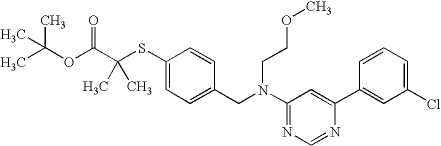

150 mg of the compound from Example 9A (0.33 mmol), 73 mg of 3-chlorophenylboronic acid

(0.46 mmol), 92 mg of potassium carbonate (0.66 mmol) and 15 mg of tetrakis(triphenylphosphine)palladium(0) (0.01 mmol) are dissolved in 5 ml of 1,2-dimethoxyethane/ethanol (4:1), and 1.7 ml of water are added. In a pressure vessel, the mixture is then heated in a microwave to 140° C. for 30 min. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 110 mg (63% of theory) of the title compound.

LC/MS (method 3): Rₜ=3.29 min; MS (ESIpos): m/z=528 [M+H]⁺.

### Example 14A

### tert-Butyl 2-[(4-{[[6-(3-methylphenyl)pyrimidin-4-yl](2-methoxyethyl)amino]methyl}phenyl)thio]-2-methylpropanoate

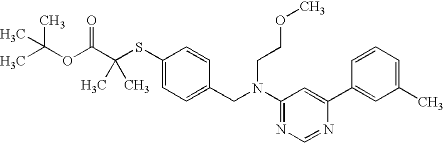

261 mg of the compound from Example 9A (0.58 mmol), 110 mg of 3-methylphenylboronic acid (0.81 mmol), 160 mg of potassium carbonate (1.16 mmol) and 27 mg of tetrakis(triphenylphosphine)palladium(0) (0.02 mmol) are dissolved in 6 ml of 1,2-dimethoxyethane/ethanol (4:1), and 2 ml of water are added. In a pressure vessel, the mixture is then heated in a microwave at 140° C. for 30 min. The mixture is then diluted with water and extracted twice with ethyl acetate. The combined organic phases are dried with sodium sulfate and the solvent is distilled off under reduced pressure. Work-is carried out by preparative HPLC (mobile phase: acetonitrile/water with 0.1% formic acid, gradient 20:80→95:5). This gives 129 mg (44% of theory) of the title compound.

LC/MS (method 2): Rₜ=2.86 min; MS (ESIpos): m/z=508 [M+H]⁺.

### Example 15A

### tert-Butyl 2-[(4-{[[6-(4-methylphenyl)pyrimidin-4-yl](2-methoxyethyl)amino]methyl}phenyl)thio]-2-methylpropanoate

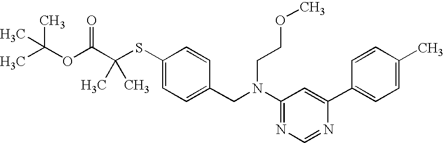

250 mg of the compound from Example 9A (0.58 mmol), 110 mg of 4-methylphenylboronic acid (0.81 mmol), 160 mg of potassium carbonate (1.16 mmol) and 27 mg of tetrakis(triphenylphosphine)palladium(0) (0.02 mmol) are dissolved in 6 ml of 1,2-dimethoxyethane/ethanol (4:1), and 2 ml of water are added. In a pressure vessel, the mixture is then heated in a microwave at 140° C. for 30 min. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 154 mg (51% of theory) of the title compound.

LC/MS (method 3): Rₜ=2.80 min; MS (ESIpos): m/z=508 [M+H]⁺.

¹H-NMR (300 MHz, DMSO-d₆): δ [ppm]=1.29 (s, 9H), 1.34 (s, 6H), 2.35 (s, 3H), 3.24 (s, 3H), 3.55 (t, 2H), 3.81 (br. s*, 2H), 4.93 (s, 2H), 7.09 (br. s, 1H), 7.26 (t*, 4H), 7.42 (d, 2H), 7.94 (br. s, 2H), 8.55 (s, 1H).

### Example 16A

### tert-Butyl 2-({4-[((2-furylmethyl) {6-[3-(trifluoromethyl)phenyl]pyrimidin-4-yl}amino)methyl]-phenyl}thio)-2-methylpropanoate

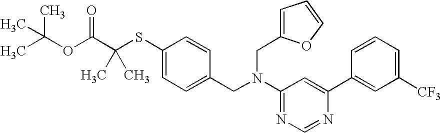

150 mg of the compound from Example 6A (0.32 mmol), 84 mg of 3-trifluoromethylphenylboronic acid (0.44 mmol), 87 mg of potassium carbonate (0.63 mmol) and 15 mg of tetrakis(triphenylphosphine)palladium(0) (0.01 μmol) are dissolved in 5 ml of 1,2-dimethoxyethane/ethanol (4:1), and 1.7 ml of water are added. In a pressure vessel, the mixture is then heated in a microwave at 140° C. for 30 min. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80 →95:5). This gives 107 mg (58% of theory) of the title compound.

LC/MS (method 1): Rₜ=3.25 min; MS (ESIpos): m/z=584 [M+H]⁺.

¹H-NMR (400 MHz, DMSO-d₆): δ [ppm]=1.29 (br. s, 9H), 1.34 (s, 6H), 4.93 (s, 4H), 6.39 (s*, 2H), 7.24 (d, 2H), 7.18-7.76 (m, 1H), 7.40 (d, 2H), 7.58 (s, 1H), 7.75 (t, 1H), 7.86 (d, 1H), 8.40 (br. s, 2H), 8.66 (s, 1H).

### Example 17A

### tert-Butyl 2-({4-[((2-furylmethyl) {6-[3-chlorophenyl]pyrimidin-4-yl}amino)methyl]phenyl}thio)-2-methylpropanoate

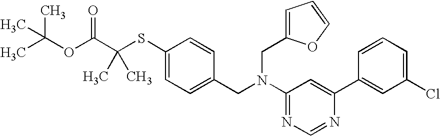

200 mg of the compound from Example 6A (0.42 mmol), 92 mg of 3-chlorophenylboronic acid

(0.59 mmol), 117 mg of potassium carbonate (0.84 mmol) and 20 mg of tetrakis(triphenylphosphine)palladium(0) (0.02 mmol) are dissolved in 6 ml of 1,2-dimethoxyethane/ethanol (4:1), and 2 ml of water are added. The mixture is then stirred under reflux overnight. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 160 mg (68% of theory) of the title compound.

LC/MS (method 3): Rₜ=3.41 min; MS (ESIpos): m/z=550 [M+H]⁺.

¹H-NMR (400 MHz, DMSO-d₆): δ [ppm]=1.29 (br. s, 9H), 1.34 (s, 6H); 4.85-4.96 (m, 4H), 6.38 (s*, 2H), 7.20-7.54 (m, 1H), 7.23 (d, 2H), 7.40 (d, 2H), 7.49-7.60 (m, 3H), 8.05 (br. s, 1H), 8.14 (br. s, 1H), 8.63 (s, 1H).

### Example 18A

### tert-Butyl 2-methyl-2-{[4-({[6-(3-methylphenyl)pyrimidin-4-yl]amino}methyl)phenyl]thio}-propanoate

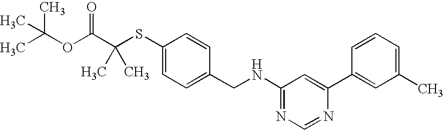

200 mg of the compound from Example 7A (0.51 mmol), 97 mg of 3-methylphenylboronic acid (0.71 mmol), 140 mg of potassium carbonate (1.02 mmol) and 23 mg tetrakis(triphenylphosphine)palladium(0) (0.02 mmol) are dissolved in 6 ml of 1,2-dimethoxyethane/ethanol (4:1), and 2 ml of water are added. The mixture is then stirred under reflux overnight. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 154 mg (63% of theory) of the title compound.

LC/MS (method 2): Rₜ=2.60 min; MS (ESIpos): m/z=450 [M+H]⁺.

¹H-NMR (300 MHz, DMSO-d₆): δ [ppm]=1.23 (s, 9H), 1.35 (s, 6H), 2.38 (s, 3H), 4.61 (d, 2H), 6.98 (br. s, 1H), 7.25-7.47 (m, 6H), 7.75 (d, 1H), 7.81 (s, 1H), 7.98 (m, 1H), 8.48 (s, 1H).

### Example 19A

### tert-Butyl 2-[(4-{[[6-(4-fluoro-3-methylphenyl)pyrimidin-4-yl](2-furylmethyl)amino]methyl}-phenyl)thio]-2-methylpropanoate

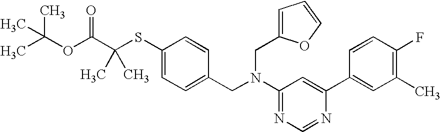

200 mg of the compound from Example 6A (0.42 mmol), 91 mg of 4-fluoro-3-methylphenylboronic acid (0.59 mmol), 117 mg of potassium carbonate (0.84 mmol) and 20 mg of tetrakis(triphenylphosphine)palladium(0) (0.02 mmol) are dissolved in 6 ml of 1,2-dimethoxyethane/ethanol (4:1), and 2 ml of water are added. The mixture is then stirred under reflux overnight. The mixture is then diluted with water and extracted twice with ethyl acetate. The combined organic phases are dried with sodium sulfate, and the solvent is distilled off under reduced pressure. Work-up is carried out by preparative HPLC (mobile phase: acetonitrile/water with 0.1% formic acid, gradient 20:80→95:5). This gives 67 mg (26% of theory) of the title compound.

LC/MS (method 1): Rₜ=3.14 min; MS (ESIpos): m/z=548 [M+H]⁺.

¹H-NMR (400 MHz, DMSO-d₆): δ [ppm]=1.30 (br. s, 9H), 1.34 (s, 6H), 2.30 (s, 3H), 4.82-4.95 (m, 4H), 6.38 (s*, 2H), 7.18-7.34 (m, 4H), 7.40 (d, 2H), 7.58 (s, 1H), 7.94 (br. s, 1H), 8.03 (br. s, 1H), 8.60 (s, 1H).

The compounds 20A-23A listed in Table 1 below, like the intermediates required for the synthesis, are obtained analogously to the examples described above:

TABLE 1

| | | | | Rₜ |
|---|---|---|---|---|
| | | Yield | | [min] |
| Example | | [% of | MS: m/z | (LC/MS |
| No. | Structure | theory] | [M + H]⁺ | method) |
| 20A | 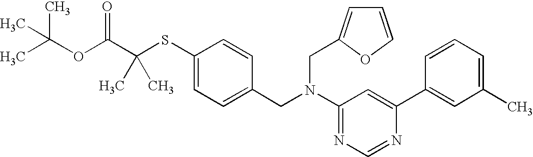 | 43 | 530 | 3.02(1) |
| 21A | 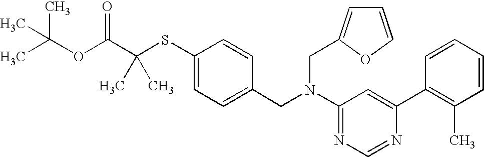 | 72 | 530 | 3.02(2) |
| 22A | 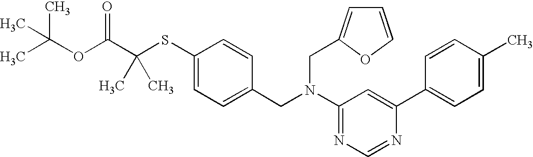 | 71 | 530 | 2.99(1) |
| 23A | 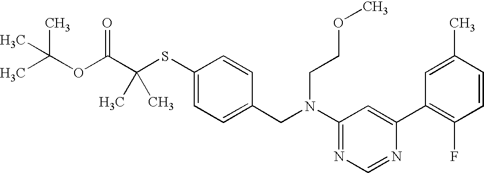 | 40 | 526 | 2.98(1) |

### Example 24A

### tert-Butyl 2-[(4-{[(2-chlorop din-4-yl)(2-methoxyethyl)amino]methyl}phenyl)thio]-2-methylpropanoate

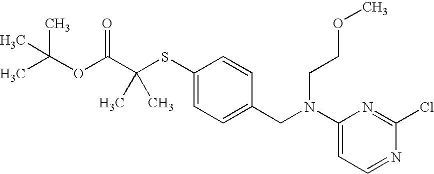

1.0 g of the compound from Example 8A (2.95 mmol), 482 mg of 2,4-dichloropyrimidine (3.24 mmol), 0.51 ml of DIEA (2.95 mmol) and 0.82 ml of triethylamine (5.89 mmol) are initially charged in 20 ml of isopropanol and reacted at 60° C. overnight. Water is added, and the mixture is extracted twice with ethyl acetate. The combined organic phases are dried with sodium sulfate and the solvent is distilled off under reduced pressure. The residue is purified by flash chromatography (silica gel, mobile phase: cyclohexane/ethyl acetate 5:1). This gives 450 mg (34% of theory) of the title compound.

LC/MS (method 1): Rₜ=2.89 min; MS (ESIpos): m/z=452 [M+H]⁺.

¹H-NMR (400 MHz, DMSO-d₆): δ [ppm]=1.31 (s, 9H), 1.35 (s, 6H), 3.22 (s, 3H), 3.51 (t, 2H), 3.52-3.87 (m, 2H), 4.82 (br. s, 2H), 6.44-6.93 (m, 1H), 7.23 (d, 2H), 7.42 (d, 2H), 8.04 (br. s, 1H).

### Example 25A

### tert-Butyl 2-({4-[((2-methoxyethyl) {2-[3-(trifluoromethyl)phenyl]pyrimidin-4-yl}amino)methyl]-phenyl}thio)-2-methylpropanoate

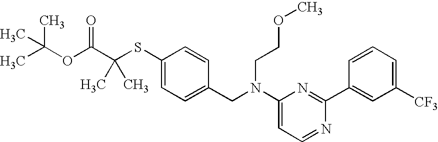

150 mg of the compound from Example 24A (0.33 mmol), 88 mg of 3-trifluoromethylphenylboronic acid (0.46 mmol), 92 mg of potassium carbonate (0.66 mmol) and 15 mg of tetrakis-(triphenylphosphine)palladium(0) (0.01 mmol) are dissolved in 6 ml of 1,2-dimethoxyethane/ethanol (4:1), and 2 ml of water are added. In a pressure vessel, the mixture is then heated in a microwave at 140° C. for 1 h. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 112 mg (60% of theory) of the title compound.

LC/MS (method 3): Rₜ=3.35 min; MS (ESIpos): m/z=562 [M+H]⁺.

¹H-NMR (400 MHz, DMSO-d₆): δ [ppm]=1.27 (s, 9H), 1.33 (s, 6H), 3.26 (s, 3H), 3.59 (t, 2H), 3.68-4.08 (m, 2H), 4.71-5.32 (br. s, 2H), 6.44-6.93 (m, 1H), 7.29 (d, 2H), 7.41 (d, 2H), 7.70 (br. s, 1H), 7.83 (m, 1H), 8.31 (br. s, 1H), 8.51 (m, 2H).

### Example 26A

### tert-Butyl 2-({4-[((2-methoxyethyl) {2-[3-methylphenyl]pyrimidin-4-yl}amino)methyl]phenyl}-thio)-2-methylpropanoate

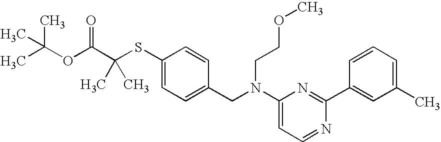

150 mg of the compound from Example 24A (0.33 mmol), 63 mg of 3-methylphenylboronic acid (0.46 mmol), 92 mg of potassium carbonate (0.66 mmol) and 15 mg of tetrakis(triphenylphosphine)palladium(0) (0.01 mmol) are dissolved in 6 ml of 1,2-dimethoxyethane/ethanol (4:1), and 2 ml of water are added. In a pressure vessel, the mixture is then heated in a microwave at 140° C. for 1 h. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 94 mg (56% of theory) of the title compound.

LC/MS (method 1): Rₜ=2.52 min; MS (ESIpos): m/z=508 [M+H]⁺.

### Example 27A

### tert-Butyl 2-({4-[((2-methoxyethyl) {2-[3-chlorophenyl]pyrimidin-4-yl}amino)methyl]phenyl}-thio)-2-methylpropanoate

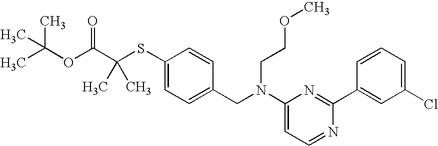

150 mg of the compound from Example 24A (0.33 mmol), 73 mg 3-chlorophenylboronic acid

(0.46 mmol), 92 mg of potassium carbonate (0.66 mmol) and 15 mg of tetrakis(triphenylphosphine)palladium(0) (0.01 mmol) are dissolved in 6 ml of 1,2-dimethoxyethane/ethanol (4:1), and 2 ml of water are added. In a pressure vessel, the mixture is then heated in a microwave at 140° C. for 1 h. The mixture is then diluted with water and extracted twice with ethyl acetate. The combined organic phases are dried with sodium sulfate and the solvent is distilled off under reduced pressure. Work-up is carried out by preparative HPLC (mobile phase: acetonitrile/water with 0.1% formic acid, gradient 20:80→95:5). This gives 78 mg (45% of theory) of the title compound.

LC/MS (method 1): Rₜ=3.12 min; MS (ESIpos): m/z=528 [M+H]⁺.

### Example 28A

### tert-Butyl 2-{[4-({(2-furylmethyl)[6-(4-methylphenoxy)pyrimidin-4-yl]amino}methyl)phenyl]-thio}-2-methylpropanoate

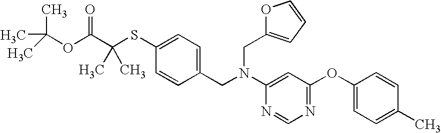

100 mg of the compound from Example 6A (0.21 mmol) are initially charged in 5 ml of abs. DMF, and 5.1 mg of sodium hydride (0.21 mmol) are added at 0° C. After 30 minutes of stirring at RT, 25.1 mg of 4-methylphenol (0.23 mmol) are added as a solution in 1 ml of abs. DMF, and the reaction mixture is stirred at RT for 12 d and at reflux temperature for 3 of theorye mixture is then poured into water and extracted twice with ethyl acetate. The crude product is purified by preparative HPLC (mobile phase: acetonitrile/water with 0.1% formic acid, gradient 20:80-95:5). This gives 55 mg (48% of theory) of the title compound.

LC/MS (method 1): Rₜ=3.20 min; MS (ESIpos): m/z=546 [M+H]⁺.

¹H-NMR (400 MHz, DMSO-d₆): δ [ppm]=1.33 (s, 9H), 1.36 (s, 6H), 2.31 (s, 3H), 4.79 (br. s, 4H), 6.11 (br. s, 1H), 6.31 (d, 1H), 6.38 (dd, 1H), 6.95 (d, 2H), 7.18 (m, 4H), 7.39 (d, 2H), 7.58 (d, 1H), 8.21 (s, 1H).

### Example 29A

### tert-Butyl 2-{[4-({(2-furylmethyl)[6-phenoxypyrimidin-4-yl]amino}methyl)phenyl]thio}-2-methylpropanoate

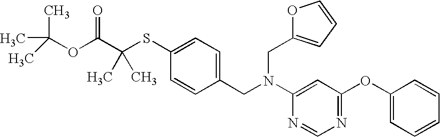

175 mg of tert-butyl 2-[(4-{[(2-furylmethyl)amino]methyl}phenyl)thio]-2-methylpropanoate hydrochloride (25.13 mmol) [prepared according to WO 02/28821, Example II-3] are initially charged in 10 ml of abs. ethanol. 0.08 ml of DIEA (0.48 mmol) and 0.13 ml of triethylamine (0.97 mmol) are then added. 100 mg of 4-chloro-6-phenoxypyrimidine (0.48 mmol) [preparation see Vainilavichyus et al., Pharm. Chem. J. 23, 500-503 (1989)] are then added, and the reaction mixture is stirred at reflux temperature for 2 d. The mixture is then concentrated, taken up in 5 ml of abs. DMF and heated at reflux temperature for another 2 d. The solvent is distilled off under reduced pressure and the residue is subsequently purified by preparative HPLC (mobile phase: acetonitrile/water with 0.1% formic acid, gradient 20:80→95:5). This gives 31 mg (12% of theory) of the title compound.

LC/MS (method 2): Rₜ=3.33 min; MS (ESIpos): m/z=532 [M+H]⁺.

### Example 30A

### tert-Butyl 2-methyl-2-({4-[(prop-2-yn-1-ylamino)methyl]phenyl}thio)propanoate

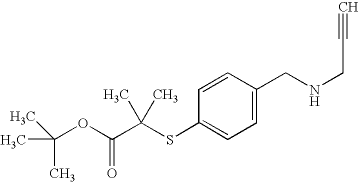

5.00 g of tert-butyl 2-{[4-(aminomethyl)phenyl]thio}-2-methylpropanoate hydrochloride (Example 34A, 15.73 mmol) are initially charged in 50 ml of DMF, and 1.87 g of 3-bromo-1-propyne (15.73 mmol), 5.48 ml of triethylamine (39.32 mmol) and 0.58 g of TBAI (1.57 mmol) are then added at RT. The mixture is stirred at RT overnight and then taken up in water and ethyl acetate. The aqueous phase is extracted three times with ethyl acetate and the organic phases are combined and then washed with saturated sodium chloride solution. After drying with sodium sulfate, the solvent is removed under reduced pressure. Work-up is carried out by flash chromatography (silica gel, mobile phase: cyclohexane/ethyl acetate 5:1→6:4). This gives 1.70 g (34% of theory) of the title compound.

LC/MS (method 2): Rₜ=1.84 min; MS (ESIpos): m/z=320 [M+H]⁺.

¹H-NMR (400 MHz, DMSO-d₆): δ [ppm]=1.35 (s*, 15H), 2.56 (br. s, 1H), 3.09 (t, 1H), 3.26 (d, 2H), 3.75 (s, 2H), 7.23 (d, 2H), 7.40 (d, 2H).

### Example 31A

### 4-Chloro-6-(3-chlorophenyl)pyrimidine

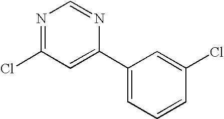

663 mg of 4,6-dichloropyrimidine (4.45 mmol), 696 mg of 3-chlorophenylboronic acid (4.45 mmol), 1.23 g of potassium carbonate (8.90 mmol) and 36 mg of [1,1′-bis(diphenylphosphino)ferrocene]dichloropalladium(II) dichloromethane complex are initially charged in 33 ml of 1,2-dimethoxyethane/water (10:1). The reaction mixture is stirred at RT overnight and then taken up in water and ethyl acetate. The aqueous phase is extracted twice with dichloromethane and the organic phases are combined and then dried with sodium sulfate. The solvent is distilled off under reduced pressure and the residue is purified by preparative HPLC (mobile phase: acetonitrile/water with 0.1% formic acid, gradient 20:80→95:5). This gives 420 mg (42% of theory) of the title compound.

LC/MS (method 3): Rₜ=2.67 min; MS (ESIpos): nl/z=225 [M+H]⁺.

¹H-NMR (400 MHz, DMSO-d₆): δ [ppm]=7.61 (t, 1H), 7.67 (d, 1H), 8.23 (d, 1H), 8.31 (m, 1H), 8.42 (s, 1H), 9.13 (s, 1H).

### Example 32A

### tert-Butyl 2-[(4-{[[6-(3-chlorophenyl)pyrimidin-4-yl](prop-2-yn-1-yl)amino]methyl}phenyl)thio]-2-methylpropanoate

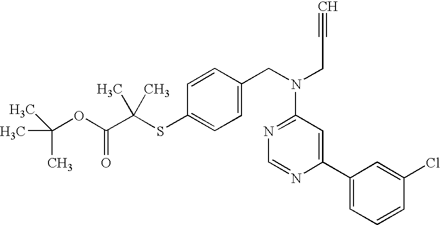

142 mg of the compound from Example 30A (0.44 mmol), 100 mg of the compound from Example 31A (0.44 mmol) and 0.12 ml of DIEA (0.67 mmol) in 2 ml of dioxane are reacted at 120° C. in a pressure vessel overnight. The solvent is distilled off under reduced pressure and the residue is then purified by preparative HPLC (mobile phase: acetonitrile/water with 0.1% formic acid, gradient 20:80→95:5). This gives 62 mg (28% of theory) of the title compound.

LC/MS (method 2): Rₜ=3.33 min; MS (ESIpos): m/z=508 [M+H]⁺.

¹H-NMR (400 MHz, DMSO-d₆): δ [ppm]=1.29 (s, 9H), 1.35 (s, 6H), 3.22 (t, 1H), 4.50 (d, 2H), 4.97 (s, 2H), 7.29-7.35 (m, 3H), 7.43 (d, 2H), 7.50-7.60 (m, 2H), 8.07 (d, 1H), 8.16 (s, 1H), 8.66 (s, 1H).

### Example 33A

### tert-Butyl 2-(4-cyanophenylsulfanyl)-2-methylpropanoate

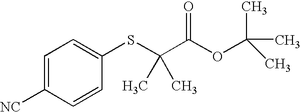

In a 26-liter tank, 2473 g (19.01 mol) of sodium sulfide (contains water) are suspended in 14.4 liters of NMP. 5.1 liters of the solvent are then removed again by distillation at 125-130° C. and 110 mbar. At an internal temperature of 130-140° C., a solution of 2110 g (15.33 mol) of 4-chlorobenzonitrile in 3.84 litres of NMP is then added dropwise over a period of one hour. The temperature is increased to 155-160° C., and the mixture is stirred for another 6 h. At 40-45° C., 3761 g (16.86 mol) of tert-butyl bromisobutyrate are metered in over a period of 45 min. At 97° C. and 24 mbar, 13.0 liters of the solvent are then distilled off, the mixture is cooled to 90° C. and 5.8 liters of methylcyclohexane are added. The mixture is cooled to 15-20° C., 7.70 liters of water and 288 g of kieselguhr are added, and the mixture is stirred at 20° C. for 15 min. The mixture is then filtered through a porcelain nutsch with a Seitz filter plate (K800), the filtrate is transferred into a 40-liter separating funnel and the phases are separated. Twice, the organic phase (9.1 liters) is stirred with in each case 5.8 liters of water, and the organic phase is concentrated on a rotary evaporator at 55-60° C./1 mbar. The residue obtained is 3788 g (89% of theory) of an oil which solidifies on storage at room temperature (purity according to GC 93%). The residue is used for the next step without further purification.

¹H-NMR (500 MHz, DMSO-d₆): δ=1.37 (s, 9H), 1.45 (s, 6H), 7.60 (d, 2H), 7.85 (d, 2H).

### Example 34A

### tert-Butyl 2-[4-(aminomethyl)phenylsulfanyl]-2-methylpropanoate hydrochloride

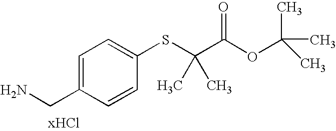

In a 26-liter tank, a solution of 2627 g (16.11 mol) of borane N,N-diethylaniline complex is, at 72° C., added dropwise over a period of 2 h to a solution of 3000 g (10.74 mol) of tert-butyl 2-(4-cyanophenylsulfanyl)-2-methylpropanoate (Example 33A) in 5.5 litres of THF. The mixture is stirred at 72° C. for 1 h and then cooled to RT, and 2.33 litres of methanol are metered in over a period of 1 h. 5.81 liters of 6 M hydrochloric acid are then added, and the mixture is stirred at RT overnight. The mixture is transferred into a 40-liter separating funnel, and the tank is rinsed with 3.88 liters of water and 7.75 liters of methylcyclohexane. Twice, the organic phase is stirred with in each case 3.8 liters of water. The combined aqueous phases are extracted with 3.88 liters of methylcyclohexane and then adjusted to pH 10.5 using concentrated aqueous sodium hydroxide solution (consumption: 2.5 liters). Twice, the aqueous/oily phase is stirred with in each case 3.88 liters of methylcyclohexane, and the combined organic phases are washed with 5.81 liters of water. Using a rotary evaporator, the organic phase (14.5 liters) is concentrated at 75° C./45 mbar. This gives 4.45 kg of a crude solution which contains the desired product as a mixture with diethylaniline.

This crude solution is combined with an earlier batch of equal size, and most of the diethylaniline is distilled off in two steps using a thin-layer evaporator (1st distillation: product feed 450 g/h, feed temperature 80-85° C., pressure 2.7 mbar, head temperature 67° C., bottom temperature 37° C.; 2nd distillation: identical conditions at 1.0 mbar). In an enamel tank, the distillation residue (3664 g) is taken up in 7.8 liters of MTBE, and a 5-6 molar solution of hydrogen chloride in isopropanol is added dropwise over a period of 20 min. During the addition, the internal temperature increases to 47° C. The suspension is cooled to RT and stirred for 2 h. It is filtered off with suction through a Seitz filter plate, and the residue is washed four times with in each case 2.6 liters of MTBE. The moist product (5.33 kg) is dried under reduced pressure and under nitrogen at 40° C. until the mass remains constant. The two combined batches give 2780 g (41% of theory) of the title compound as white crystals.

¹H-NMR (400 MHz, DMSO-d₆): δ=1.39 (m, 15H), 4.04 (s, 2H), 7.49 (m, 4H), 8.48 (br. s, 3H).

MS (DCI/NH₃): m/z=282 [M+H]⁺, 299 [M+NH₄]⁺.

### Example 35A

### tert-Butyl 2-methyl-2-[(4-{[(1,3-thiazol-2-ylmethyl)amino]methyl}phenyl)thio]propanoate

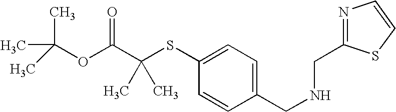

To release the base from the hydrochloride, 1.74 g of the compound from Example 34A (6.19 mmol) are taken up in 30 ml of 1 N aqueous sodium hydroxide solution, extracted with ethyl acetate and dried with sodium sulfate. The solvent is then removed using a rotary evaporator. The free base obtained in this manner is taken up in 10 ml of methanol, 700 mg of 1,3-thiazole-2-carbaldehyde (6.19 mmol) are added and the mixture is stirred at RT for about 2 h (TLC analysis) to form the imine. 234 mg of sodium borohydride (6.19 mmol) are then added, and the mixture is stirred at RT for 5 min. The solvent is distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: cyclohexane/ethyl acetate 7:3). This gives 1.26 g of the title compound (52% of theory).

LC/MS (method 2): Rₜ=1.71 min; MS (ESIpos): m/z=379 [M+H]⁺.

¹H-NMR (400 MHz, DMSO-d₆): δ [ppm]=1.35 (s, 9H), 1.36 (s, 6H), 3.78 (s*, 2H), 3.95 (s*, 2H), 7.37 (d, 2H), 7.42 (d, 2H), 7.60 (d, 1H), 7.70 (d, 1H).

### Example 36A

### tert-Butyl 2-[(4-{[(6-chloropyrimidin-4-yl)(1,3-thiazol-2-ylmethyl)amino]methyl}phenyl)thio]-2-methylpropanoate

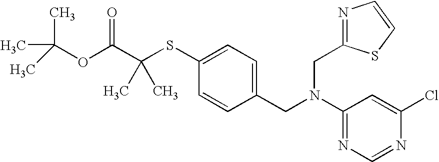

1.00 g of the compound from Example 35A (2.64 mmol) is initially charged in 10 ml of 2-propanol, and 0.69 ml of DIEA (3.96 mmol) is added. 413 mg of 4,6-dichloropyrimidine (2.77 mmol) are then added. The mixture is stirred at reflux temperature overnight. After cooling, the solvent is distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: cyclohexane/ethyl acetate 4:1). This gives 772 mg of the title compound (60% of theory).

LC/MS (method 3): Rₜ=3.08 min; MS (ESIpos): m/z=491 [M+H]⁺.

¹H-NMR (400 MHz, DMSO-d₆): δ [ppm]=1.32 (s, 9H), 1.35 (s, 6H), 4.92 (s, 2H), 5.14 (s, 2H), 6.93 (br. s, 1H), 7.26 (d, 2H), 7.42 (d, 2H), 7.66 (d, 1H), 7.76 (d, 1H), 8.44 (s, 1H).

### Example 37A

### tert-Butyl 2-methyl-2-({4-[((1,3-thiazol-2-ylmethyl) {6-[3-(trifluoromethyl)phenyl]pyrimidin-4-yl}-amino)methyl]phenyl}thio)propanoate

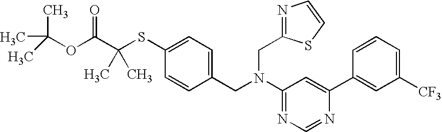

142 mg of the compound from Example 36A (0.289 mmol) and 76.8 mg of 3-trifluoromethylphenylboronic acid (0.405 mmol) are initially charged in 5 ml of DME/ethanol (4:1). 13.4 mg of tetrakis(triphenylphosphine)palladium(0) (0.012 mmol), 79.9 mg of potassium carbonate (0.578 mmol) and 1.7 ml of water are added. The reaction mixture is then stirred at 80° C. overnight. After cooling, the mixture is taken up in 10 ml of water and extracted twice with ethyl acetate. The combined organic phases are dried with sodium sulfate, and the solvent is then distilled off under reduced pressure. The residue is purified by preparative HPLC (mobile phase: acetonitrile/water with 0.1% formic acid, gradient 20:80→95:5). This gives 130 mg (75% of theory) of the title compound.

LC/MS (method 1): Rₜ=3.23 min; MS (ESIpos): m/z=601 [M+H]⁺.

### Example 38A

### tert-Butyl 2-methyl-2-{[4-({[(1-methyl-1H-imidazol-2-yl)methyl]amino}methyl)phenyl]thio}-propanoate

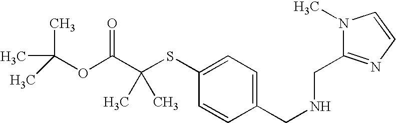

To release the base from the hydrochloride, 7.67 g of the compound from Example 34A (27.24 mmol) are taken up in 30 ml of 1 N aqueous sodium hydroxide solution, extracted with ethyl acetate and dried with sodium sulfate. The solvent is then removed using a rotary evaporator. The free base obtained in this manner is taken up in 10 ml of methanol, 3.00 g of 1-methyl-1H-imidazole-2-carbaldehyde (27.24 mmol) are added and the mixture is stirred at RT for about 2 h (TLC analysis) to form the imine. 1.031 g of sodium borohydride (27.24 mmol) are then added, and the mixture is stirred at RT for 5 min. The solvent is distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: cyclohexane/ethyl acetate 7:3). This gives 10.01 of the title compound (96% of theory).

LC/MS (method 1): Rₜ=1.55 min; MS (ESIpos): m/z=376 [M+H]⁺.

¹H-NMR (400 MHz, DMSO-d₆): δ [ppm]=1.36 (s*, 15H), 2.57 (br. s, 1H), 3.35 (s, 3H), 3.67 (s*, 2H), 3.69 (s*, 2H), 6.74 (d, 1H), 7.03 (d, 1H), 7.35 (d, 2H), 7.41 (d, 2H).

### Example 39A

### tert-Butyl 2-{[4-({(6-chloropyrimidin-4-yl)[(1-methyl-1H-imidazol-2-yl)methyl]amino}methyl)phenyl]thio}-2-methylpropanoate

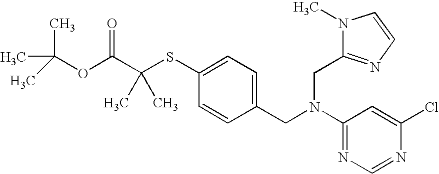

4.00 g of the compound from Example 38A (10.6 mmol) are initially charged in 50 ml of 2-propanol, and 2.78 ml of DIEA (2.07 mmol) are added. 1.67 g of 4,6-dichloropyrimidine (11.18 mmol) are then added. The reaction mixture is stirred at 50° C. overnight. After cooling, the solvent is distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: ethyl acetate→ethyl acetate/ethanol 5:1). This gives 3.90 g of the title compound (74% of theory).

LC/MS (method 1): Rₜ=1.73 min; MS (ESIpos): m/z=488 [M+H]⁺.

¹H-NMR (400 MHz, DMSO-d₆): δ [ppm]=1.32 (s, 9H), 1.35 (s, 6H), 3.56 (s, 3H), 4.85 (br. s*, 4H), 6.78 (s*, 1H), 6.92 (br. s, 1H), 7.07 (s*, 1H), 7.19 (d, 2H), 7.39 (d, 2H), 8.39 (s, 1H).

### Example 40A

### tert-Butyl 2-methyl-2-[(4-{[[(1-methyl-1H-imidazol-2-yl)methyl](6-{[4-(trifluoromethyl)phenyl]-amino}pyrimidin-4-yl)amino]methyl}phenyl)thio]propanoate

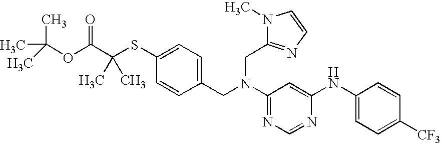

150 mg of the compound from Example 39A (0.307 mmol), 99.0 mg of 4-trifluoromethylaniline (0.615 mmol), 12.4 mg of bis(dibenzylidenacetone)palladium(0) (0.022 mmol), 18.3 mg of 1,3-bis(2,6-diisopropylphenyl)imidazolium chloride (0.043 mmol) and 103.5 mg of potassium tert-butoxide (0.922 mmol) are dissolved in 3 ml of dioxane and reacted at 120° C. overnight. The reaction mixture is then taken up in water, acidified with glacial acetic acid and extracted twice with ethyl acetate. The combined organic phases are dried with sodium sulfate, the solvent is removed using a rotary evaporator and the residue is purified by HPLC (mobile phase: acetonitrile/water with 0.1% formic acid, gradient 20:80→95:5). This gives 50 mg (25% of theory) of the title compound.

LC/MS (method 3): Rₜ=2.31 min; MS (ESIpos): m/z=613 [M+H]⁺.

¹H-NMR (400 MHz, DMSO-d₆): δ [ppm]=1.33 (s, 9H), 1.35 (s, 6H), 3.61 (s, 3H), 4.75 (s, 2H), 4.87 (s, 2H), 5.97 (s, 1H), 6.78 (s*, 1H), 7.07 (s*, 1H), 7.19 (d, 2H), 7.40 (d, 2H), 7.57 (d, 2H), 7.74 (d, 2H), 8.30 (s, 1H), 9.48 (s, 1H).

### Example 41A

### tert-Butyl 2-[(4-{[(2-methoxyethyl)(6-{[3-(trifluoromethyl)phenyl]amino}pyrimidin-4-yl)amino]-methyl}phenyl)thio]-2-methylpropanoate

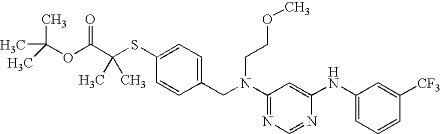

150 mg of the compound from Example 9A (0.332 mmol), 53.5 mg of 3-trifluoromethylaniline (0.332 mmol), 3.0 mg of tris(dibenzylidenacetone)dipalladium(0) (0.003 mmol), 7.9 mg of dicyclohexyl(2′,4′,6′-triisopropylbiphenyl-2-yl)phosphine (0.017 mmol) and 114.7 mg of potassium carbonate (0.830 mmol) are dissolved in 2 ml tert-butanol and heated at 200° C. in a microwave for 2 h. The reaction mixture is then filtered, the filtrate is concentrated and water is added to the residue. After two extractions with ethyl acetate, the combined organic phases are dried with sodium sulfate and the solvent is removed using a rotary evaporator. The residue is then purified by preparative HPLC (mobile phase: acetonitrile/water with 0.1% formic acid, gradient 20:80→95:5). This gives 63 mg (33% of theory) of the title compound.

LC/MS (method 1): Rₜ=3.05 min; MS (ESIpos): m/z=577 [M+H]⁺.

¹H-NMR (400 MHz, DMSO-d₆): δ [ppm]=1.32 (s, 9H), 1.35 (s, 6H), 3.23 (s, 3H), 3.50 (t, 2H), 3.67 (br. s, 2H), 4.79 (s, 2H), 5.88 (s, 1H), 7.19-7.26 (m, 3H), 7.42 (d, 2H), 7.47 (d, 1H), 7.75 (d, 1H), 8.11 (s, 1H), 8.25 (s, 1H), 9.39 (s, 1H).

### Example 42A

### 4-(Chloromethyl)-3,5-dimethylisoxazole

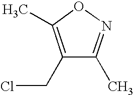

10.0 g of 3,5-dimethylisoxazole (103.0 mmol) are initially charged in 30 ml of concentrated hydrochloric acid, and 18.5 g of paraformaldehyde (615.8 mmol) are added. The reaction mixture is stirred at 70° C. overnight. After cooling, the mixture is taken up in 100 ml of water and extracted twice with ethyl acetate. The combined organic phases are dried with sodium sulfate and the solvent is removed using a rotary evaporator. The residue is purified by column chromatography (silica gel, mobile phase: cyclohexane/ethyl acetate 2:1). This gives 3.95 g of the title compound (24% of theory).

LC/MS (method 7): Rₜ=2.00 min; MS (ESIpos): m/z=128 [M+H]⁺.

¹H-NMR (400 MHz, DMSO-d₆): δ [ppm]=2.23 (s, 3H), 2.40 (s, 3H), 4.68 (s, 2H).

### Example 43A

### tert-Butyl 2-{[4-({[(3,5-dimethylisoxazol-4-yl)methyl]amino}methyl)phenyl]thio}-2-methylpropanoate

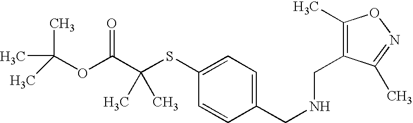

3.07 g of the compound from Example 34A (9.65 mmol) are initially charged in 15 ml of DMF, and 3.70 g of triethylamine (26.54 mmol) are added. After addition of 0.36 g of TBAI (0.97 mmol) and 1.70 g of the compound from Example 42A (11.7 mmol), the reaction mixture is stirred at RT overnight. The solvent is then distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: cyclohexane/ethyl acetate 2:1). This gives 1.29 g of the title compound (33% of theory).

LC/MS (method 2): Rₜ=1.68 min; MS (ESIpos): m/z=391 [M+H]⁺.

### Example 44A

### tert-Butyl 2-{[4-({(6-chloropyrimidin-4-yl)[(3,5-dimethylisoxazol-4-yl)methyl]amino}-2-methyl)phenyl]thio}-2-methylpropanoate

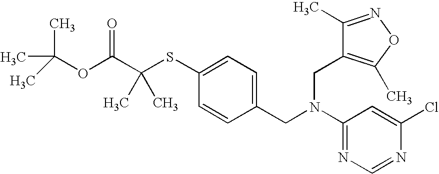

1.90 g of the compound from Example 43A (4.87 mmol) are initially charged in 15 ml of 2-propanol, and 1.27 ml of DIEA (7.30 mmol) are added. 1.09 g of 4,6-dichloropyrimidine (7.30 mmol) are then added. The reaction mixture is stirred at reflux temperature overnight. After cooling, the solvent is distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: dichloromethane). This gives 2.07 g of the title compound (85% of theory).

LC/MS (method 1): Rₜ=2.90 min; MS (ESIpos): m/z=503 [M+H]⁺.

¹H-NMR (400 MHz, DMSO-d₆): δ [ppm]=1.32 (s, 9H), 1.35 (s, 6H), 2.07 (s, 3H), 2.27 (s, 3H), 4.66 (s, 2H), 4.76 (s, 2H), 6.89 (br. s, 1H), 7.15 (d, 2H), 7.41 (d, 2H), 8.44 (s, 1H).

### Example 45A

### tert-Butyl 2-{[4-({[6-(cyclohexyloxy)pyrimidin-4-yl][(3,5-dimethylisoxazol-4-yl)methyl]amino}-methyl)phenyl]thio}-2-methylpropanoate

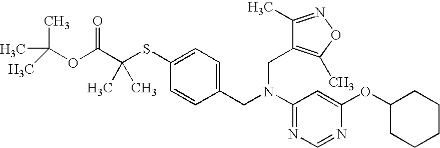

90.0 mg of cyclohexanol (0.895 mmol) are initially charged in 3 ml of DMSO, and 100 mg of potassium tert-butoxide (0.895 mmol) are added. After 15 min of stirring, 300 mg of the compound from Example 44A (0.596 mmol) are added and the mixture is then stirred at RT overnight. The mixture is taken up in water and neutralized using 1 N hydrochloric acid. After two extractions with ethyl acetate, the combined organic phases are dried with sodium sulfate and the solvent is removed using a rotary evaporator. The crude product is purified by preparative HPLC (mobile phase: acetonitrile/water with 0.1% formic acid, gradient 20:80→95:5). This gives 64 mg (19% of theory) of the title compound.

LC/MS (method 3): Rₜ=3.48 min; MS (ESIpos): m/z=567 [M+H]⁺.

### Example 46A

### tert-Butyl 2-methyl-2-{[4-({[(2-methyl-1,3-thiazol-4-yl)methyl]amino}methyl)phenyl]thio}-propanoate

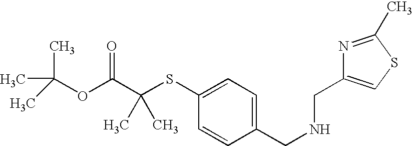

13.43 g of the compound from Example 34A (42.5 mmol) are initially charged in 60 ml of DMF, and 22.1 ml of triethylamine (158.4 mmol) are added. After addition of 1.56 g of TBAI (4.23 mmol) and 7.00 g of 4-chloromethyl-2-methylthiazolium chloride (38.02 mmol), the reaction mixture is stirred at RT overnight. The solvent is distilled off under reduced pressure and the residue is taken up in water and then made slightly basic using 1 N aqueous sodium hydroxide solution. After two extractions with ethyl acetate, the combined organic phases are dried with sodium sulfate and the solvent is removed using a rotary evaporator. The residue is worked up by column chromatography (silica gel, mobile phase: cyclohexane/ethyl acetate 1:1→5:1). This gives 7.10 g of the title compound (39% of theory).

LC/MS (method 3): Rₜ=1.78 min; MS (ESIpos): m/z=393 [M+H]⁺.

¹H-NMR (400 MHz, DMSO-d₆): δ [ppm]=1.35 (s*, 15H), 2.62 (s, 3H), 3.70 (s, 2H), 3.74 (s, 2H), 7.21 (s, 1H), 7.35 (d, 2H), 7.40 (d, 2H).

### Example 47A

### tert-Butyl 2-{[4-({(6-chloropyrimidin-4-yl)[(2-methyl-1,3-thiazol-4-yl)methyl]amino}methyl)phenyl]thio}-2-methylpropanoate

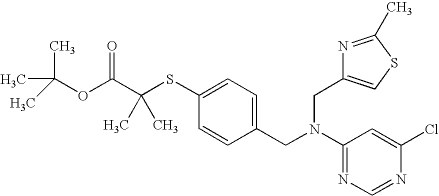

7.10 g of the compound from Example 46A (16.28 mmol) are initially charged in 100 ml of 2-propanol, and 4.25 ml of DIEA (24.42 mmol) are added. 2.55 g of 4,6-dichloropyrimidine

(17.09 mmol) are then added. The reaction mixture is stirred at reflux temperature overnight. After cooling, the solvent is distilled off under reduced pressure and the residue is taken up in water. After two extractions with ethyl acetate, the combined organic phases are dried with sodium sulfate and the solvent is removed using a rotary evaporator. The residue is worked up by column chromatography (silica gel, mobile phase: cyclohexane/ethyl acetate 4:1). This gives 9.0 g of the title compound (96% of theory).

LC/MS (method 2): Rₜ=3.15 min; MS (ESIpos): m/z=505 [M+H]⁺.

¹H-NMR (400 MHz, DMSO-d₆): δ [ppm]=1.32 (s, 9H), 1.35 (s, 6H), 2.61 (s, 3H), 4.54-5.04 (m, 4H), 6.62-7.10 (m, 1H), 7.23 (d, 2H), 7.31 (s, 1H), 7.40 (d, 2H), 8.37 (s, 1H).

### Example 48A

### tert-Butyl 2-{[4-({[(2,4-dimethyl-1,3-thiazol-5-yl)methyl]amino}methyl)phenyl]thio}-2-methylpropanoate

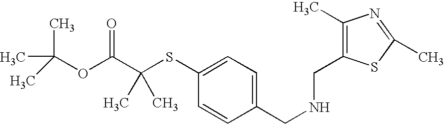

To release the base from the hydrochloride, 2.20 g of the compound from Example 34A (7.82 mmol) are taken up in 30 ml of 1 N aqueous sodium hydroxide solution, extracted with ethyl acetate and dried with sodium sulfate. The solvent is then removed using a rotary evaporator. The free base obtained in this manner is taken up in 15 ml of methanol, 1.10 g of 2,4-dimethyl-1,3-thiazole-5-carbaldehyde (7.82 mmol) are added and the mixture is stirred at RT for about 2 h (TLC analysis) to form the imine. 296 mg of sodium borohydride (7.82 mmol) are then added, and the mixture is stirred at RT for 5 min. The solvent is distilled off under reduced pressure and the residue is taken up in water. After two extractions with ethyl acetate, the combined organic phases are dried with sodium sulfate and the solvent is removed using a rotary evaporator. This gives 2.80 g of the title compound (86% of theory) in a purity of 90% (LC/MS) which are used without further purification for the next step.

LC/MS (method 1): Rₜ=1.50 min; MS (ESIpos): m/z=407 [M+H]⁺.

### Example 49A

### tert-Butyl 2-{[4-({(6-chloropyrimidin-4-yl)[(2,4-dimethyl-1,3-thiazol-5-yl)methyl]amino}methyl)phenyl]thio}-2-methylpropanoate

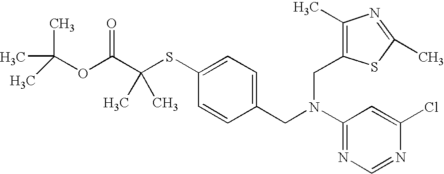

2.80 g of the compound from Example 48A (6.89 mmol) and 1.08 g of 4,6-dichloropyrimidine

(7.23 mmol) are initially charged in 50 ml of 2-propanol, and 1.80 ml of DIEA (10.33 mmol) are added. The reaction mixture is then stirred at 50° C. overnight. The solvent is distilled off under reduced pressure and the residue is taken up in water. After two extractions with ethyl acetate, the combined organic phases are dried with sodium sulfate and the solvent is removed using a rotary evaporator. The residue is purified by column chromatography (silica gel, mobile phase: cyclohexane/ethyl acetate 7:3). This gives 2.39 g of the title compound (65% of theory).

LC/MS (method 3): Rₜ=3.11 min; MS (ESIpos): m/z=519 [M+H]⁺.

¹H-NMR (400 MHz, DMSO-d₆): δ [ppm]=1.32 (s, 9H), 1.35 (s, 6H), 2.09 (s, 3H), 2.22 (s, 3H), 4.77 (br. s, 2H), 4.89 (br. s, 2H), 6.85 (br. s, 1H), 7.20 (d, 2H), 7.40 (d, 2H), 8.45 (s, 1H).

### Example 50A

### tert-Butyl 2-{[4-({[6-(cyclohexyloxy)pyrimidin-4-yl][(2,4-dimethyl-1,3-thiazol-5-yl)methyl]-amino}methyl)phenyl]thio}-2-methylpropanoate

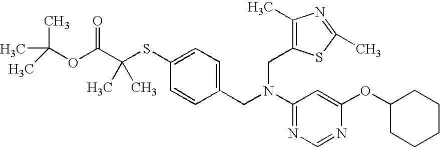

Analogously to the preparation of Example 45A, 150 mg of the compound from Example 49A (0.29 mmol), 43.4 mg of cyclohexanol (0.43 mmol) and 48.9 mg of potassium tert-butoxide (0.44 mmol) give 48 mg of the title compound (29% of theory).

LC/MS (method 3): Rₜ=3.55 min; MS (ESIpos): m/z=583 [M+H]⁺.

¹H-NMR (400 MHz, DMSO-d₆): δ [ppm]=1.20 (m, 2H), 1.26-1.39 (m, 2H), 1.31 (s, 9H), 1.35 (s, 6H), 1.51 (m, 2H), 1.68 (m, 2H), 1.87 (m, 2H), 2.21 (s, 3H), 4.67 (br. s, 2H), 4.85 (br. s, 2H), 4.92 (m, 1H), 5.81 (br. s, 1H), 7.19 (d, 2H), 7.40 (d, 2H), 8.29 (s, 1H).

### Example 51A

### tert-Butyl 2-[(4-{[{6-[4-(4-fluorophenyl)piperazin-1-yl]pyrimidin-4-yl}(2-methoxyethyl)amino]-methyl}phenyl)thio]-2-methylpropanoate

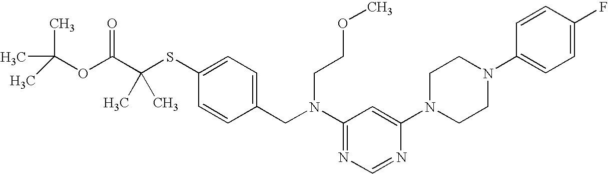

150 mg of the compound from Example 9A (0.332 mmol), 119.6 mg of 1-(4-fluorophenyl)piperazine (0.664 mmol), 13.4 mg of bis(dibenzylidenacetone)palladium(0) (0.023 mmol), 19.7 mg of 1,3-bis(2,6-diisopropylphenyl)imidazolium chloride (0.046 mmol) and 111.7 mg of potassium tert-butoxide (0.996 mmol) are dissolved in 3 ml of dioxane and reacted at 100° C. overnight. The reaction mixture is taken up in water, neutralized with 1 N hydrochloric acid and extracted with ethyl acetate. The combined organic phases are dried with sodium sulfate, the solvent is removed using a rotary evaporator and the crude product is purified by preparative HPLC (mobile phase: acetonitrile/water with 0.1% formic acid, gradient 20:80→95:5). This gives 84 mg (42% of theory) of the title compound.

LC/MS (method 3): Rₜ=2.88 min; MS (ESIpos): m/z=596 [M+H]⁺.

¹H-NMR (400 MHz, DMSO-d₆): δ [ppm]=1.32 (s, 9H), 1.34 (s, 6H), 3.10 (m, 4H), 3.22 (s, 3H), 3.49 (t, 2H), 3.62 (m, 4H), 3.66 (br. s, 2H), 4.81 (s, 2H), 5.80 (s, 1H), 6.94-7.02 (m, 2H), 7.06 (t, 2H), 7.21 (d, 2H), 7.40 (d, 2H), 8.09 (s, 1H).

### Example 52A

### tert-Butyl 2-methyl-2-({4-[([(2-methyl-1,3-thiazol-4-yl)methyl]{6-[3-(trifluoromethyl)phenoxy]-pyrimidin-4-yl}amino)methyl]phenyl}thio)propanoate

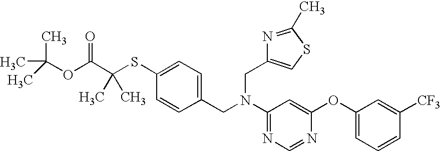

500 mg of the compound from Example 47A (0.990 mmol), 160 mg of 3-trifluoromethylphenol (0.990 mmol), 273 mg of potassium carbonate (1.980 mmol) and 118 mg of copper(II) oxide (1.485 mmol) in 4 ml of pyridine are reacted at 150° C. overnight. The reaction mixture is concentrated and the residue is taken up in ethyl acetate and then filtered through a short silica gel column using the mobile phase ethyl acetate. After concentration of the filtrate, the residue is purified by preparative HPLC (mobile phase: acetonitrile/water with 0.1% formic acid, gradient 20:80→95:5). This gives 400 mg (64% of theory) of the title compound.

LC/MS (method 2): Rₜ=3.40 min; MS (ESIpos): m/z=631 [M+H]⁺.

¹H-NMR (400 MHz, DMSO-d₆): δ [ppm]=1.33 (s, 9H), 1.36 (s, 6H), 2.62 (s, 3H), 4.45-5.15 (m, 4H), 6.31 (br. s, 1H), 7.21-7.32 (m, 3H), 7.37-7.55 (m, 4H), 7.57-7.68 (m, 2H), 8.23 (s, 1H).

### Example 53A

### tert-Butyl 2-({4-[([(3,5-dimethylisoxazol-4-yl)methyl]{6-[3-(trifluoromethyl)phenoxy]pyrimidin-4-yl}amino)methyl]phenyl}thio)-2-methylpropanoate

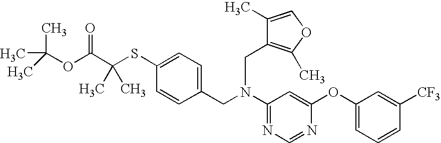

150 mg of the compound from Example 44A (0.250 mmol), 41 mg of 3-trifluoromethylphenol (0.250 mmol), 69 mg of potassium carbonate (0.501 mmol) and 30 mg of copper(II) oxide (0.376 mmol) in 3 ml of pyridine are reacted at 150° C. overnight. The reaction mixture is concentrated and the residue is taken up in ethyl acetate and then filtered through a short silica gel column using the mobile phase ethyl acetate. After concentration of the filtrate, the residue is purified by preparative HPLC (mobile phase: acetonitrile/water with 0.1% formic acid, gradient 20:80→95:5). This gives 80 mg (51% of theory) of the title compound.

LC/MS (method 3): Rₜ=3.39 min; MS (ESIpos): m/z=629 [M+H]⁺.

¹H-NMR (400 MHz, DMSO-4): δ [ppm]=1.33 (s, 9H), 1.35 (s, 6H), 2.10 (s, 3H), 2.23 (s, 3H), 4.67 (s, 2H), 4.75 (s, 2H), 6.29 (s, 1H), 7.17 (d, 2H), 7.38-7.55 (m, 6H), 8.29 (s, 1H).

### Example 54A

### tert-Butyl 2-{[4-({(2-methoxyethyl)[6-(4-methylphenoxy)pyrimidin-4-yl]amino}methyl)phenyl]-thio}-2-methylpropanoate

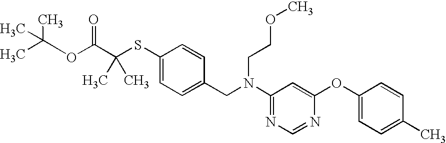

1500 mg of the compound from Example 9A (3.31 mmol), 359 mg of 4-methylphenol (3.31 mmol), 917 mg of potassium carbonate (6.64 mmol) and 396 mg copper(II) oxide (4.98 mmol) in 10 ml of pyridine are reacted at 150° C. overnight. The reaction mixture is concentrated and the residue is taken up in ethyl acetate and then filtered through a short silica gel column using the mobile phase ethyl acetate. After concentration of the filtrate, the residue is purified by preparative HPLC (mobile phase: acetonitrile/water with 0.1% formic acid, gradient 20:80→95:5). This gives 950 mg (52% of theory) of the title compound.

LC/MS (method 1): Rₜ=3.16 min; MS (ESIpos): m/z=524 [M+H]⁺.

¹H-NMR (400 MHz, DMSO-d₆): δ [ppm]=1.32 (s, 9H), 1.36 (s, 6H), 2.30 (s, 3H), 3.22 (s, 3H), 3.50 (t, 2H), 3.70 (br. s, 2H), 4.80 (br. s, 2H), 6.02 (br. s, 1H), 6.95 (d, 2H), 7.19 (t*, 4H), 7.41 (d, 2H), 8.18 (s, 1H).

### Example 55A

### tert-Butyl 2-({4-[([(3,5-dimethylisoxazol-4-yl)methyl]{6-[3-(trifluoromethyl)phenyl]pyrimidin-4-yl}amino)methyl]phenyl}thio)-2-methylpropanoate

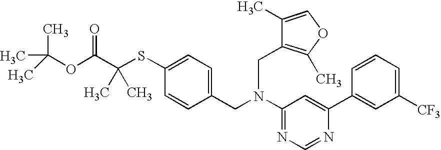

150 mg of the compound from Example 44A (0.250 mmol) and 66.6 mg of 3-trifluoromethylphenylboronic acid (0.351 mmol) are initially charged in 5 ml of DME/ethanol (4:1). After addition of 11.6 mg of tetrakis(triphenylphosphine)palladium(0) (0.010 mmol) and 69.2 mg of potassium carbonate (0.501 mmol), 1.7 ml of water are added. The reaction mixture is then stirred at 90° C. overnight. After cooling, the mixture is diluted with 10 ml of water and extracted twice with ethyl acetate. After drying of the combined organic phases over sodium sulfate, the solvent is distilled off under reduced pressure. The residue is purified by preparative HPLC (mobile phase: acetonitrile/water with 0.1% formic acid, gradient 20:80→95:5). This gives 44 mg (29% of theory) of the title compound.

LC/MS (method 3): Rₜ=3.39 min; MS (ESIpos): m/z=613 [M+H]⁺.

¹H-NMR (400 MHz, DMSO-d₆): δ [ppm]=1.27 (s, 9H), 1:33 (s, 6H), 2.10 (s, 3H), 2.25 (s, 3H), 4.75 (s, 2H), 4.87 (s, 2H), 7.20 (d, 2H), 7.41 (d*, 3H), 7.73 (t, 1H), 7.84 (d, 1H), 8.35-8.45 (m, 2H), 8.70 (s, 1HH).

### Example 56A

### tert-Butyl 2-methyl-2-({4-[({6-[3-(trifluoromethyl)phenyl]pyrimidin-4-yl}amino)methyl]phenyl}-thio)propanoate

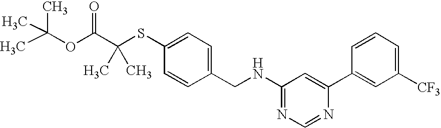

3.25 g of the compound from Example 7A (8.25 mmol), 2.19 g of 3-trifluoromethylphenylboronic acid (11.55 mmol), 2.28 g of potassium carbonate (16.5 mmol) and 381 mg of tetrakis(triphenylphosphine)palladium(0) (0.330 mmol) are dissolved in 75 ml of DME/ethanol (4:1), and 25 ml of water are added. The reaction mixture is then stirred under reflux overnight. The mixture is then diluted with water and extracted twice with ethyl acetate. The combined organic phases are dried with sodium sulfate and the solvent is removed under reduced pressure. This gives 3.50 g (78% of theory) of the title compound in a purity of 92% (LC/MS).

LC/MS (method 2): Rₜ=2.80 nin; MS (ESIpos): m/z=504 [M+H]⁺.

### Example 57A

### tert-Butyl 2-({4-[((2-fluoroethyl) {6-[3-(trifluoromethyl)phenyl]pyrimidin-4-yl}amino)methyl]-phenyl}thio)-2-methylpropanoate

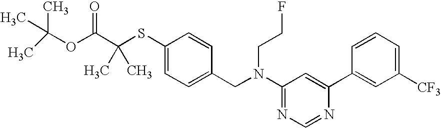

150 mg of the compound from Example 56A (0.274 mmol) are initially charged in 3 ml of abs. DMF, 11.0 mg of sodium hydride (0.274 mmol, 60% dispersion in mineral oil) are added and the mixture is stirred at RT for 30 min. 52.2 mg of 1-bromo-2-fluoroethane (0.411 mmol) are then added, and the reaction mixture is stirred at RT overnight. Work-up is carried out directly by means of preparative HPLC (mobile phase: acetonitrile/water with 0.1% formic acid, gradient 20:80→95:5). This gives 87 mg (55% of theory) of the title compound.

LC/MS (method 3): Rₜ=3.32 min; MS (ESIpos): m/z=550 [M+H]⁺.

¹H-NMR (300 MHz, DMSO-d₆): δ [ppm]=1.27 (s, 9H), 1.34 (s, 6H), 4.01 (d, 2H), 4.68 (dt, 2H), 5.00 (s, 2H), 7.22-7.48 (br. s, 1H), 7.28 (d, 2H), 7.43 (d, 2H), 7.73 (t, 1H), 7.85 (d, 1H), 8.40 (br. s, 2H), 8.64 (s, 1H).

The compounds 58A-87A of the general formula (A) listed in Table 2 below are, like the intermediates required for the synthesis, obtained analogously to the examples described above:

TABLE 2

| | |
|---|---|
| | (A) |
| 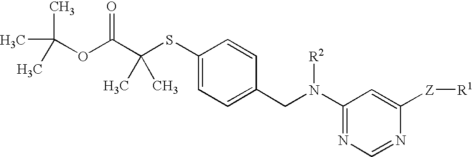 | |

| | Synthesis | | | | | |
|---|---|---|---|---|---|---|
| | analogously to | | | | | Rₜ |
| | Example No. | | | Yield | | [min] |
| Example | (from | | | [% of | MS: m/z | (LC/MS |
| No. | Example No.) | R²— | R¹—Z— | theory] | [M + H]⁺ | method) |
| 58A | 45A (9A) | 2-methoxyethyl | 4-trifluoromethyl- | 49 | 584 | 3.43 |
| | | | cyclohexyloxy | | | (2) |
| 59A | 45A (44A) | (3,5-dimethyl- | trans-4-methoxy- | 24 | 597 | 3.11 |
| | | isoxazol-4-yl)- | cyclohexyloxy | | | (1) |
| | | methyl | | | | |
| 60A | 45A (44A) | (3,5-dimethyl- | trans-4-methyl- | 21 | 581 | 3.58 |
| | | isoxazol-4-yl)- | cyclohexyloxy | | | (3) |
| | | methyl | | | | |
| 61A | 45A (49A) | (2,4-dimethyl-1,3- | trans-4-methyl- | 27 | 597 | 3.63 |
| | | thiazol-5-yl)- | cyclohexyloxy | | | (2) |
| | | methyl | | | | |
| 62A | 45A (49A) | (2,4-dimethyl-1,3- | trans-4-methoxy- | 24 | 613 | 3.26 |
| | | thiazol-5-yl)- | cyclohexyloxy | | | (2) |
| | | methyl | | | | |
| 63A | 52A (9A) | 2-methoxyethyl | 4-(trifluoro- | 25 | 578 | 3.25 |
| | | | methyl)phenoxy | | | (1) |
| 64A | 52A (47A) | (2-methyl-1,3- | 4-(trifluoro- | 22 | 631 | 3.27 |
| | | thiazol-4-yl)- | methyl)phenoxy | | | (1) |
| | | methyl | | | | |
| 65A | 45A (36A) | (1,3-thiazol-2-yl)- | 4-methylphenoxy | 18 | 563 | 3.28 |
| | | methyl | | | | (2) |
| 66A | 52A (39A) | (1-methyl-1H- | 3-(trifluoro- | 55 | 614 | 2.28 |
| | | imidazol-2- | methyl)phenoxy | | | (3) |
| | | yl)methyl | | | | |
| 67A | 52A (39A) | (1-methyl-1H- | 4-(trifluoro- | 34 | 614 | 2.29 |
| | | imidazol-2-yl)- | methyl)phenoxy | | | (3) |
| | | methyl | | | | |
| 68A | 52A (9A) | 2-methoxyethyl | 3-(trifluoro- | 43 | 578 | 3.39 |
| | | | methyl)phenoxy | | | (2) |
| 69A | 52A (39A) | (3,5-dimethyl- | 4-methylphenoxy | 24 | 575 | 3.33 |
| | | isoxazol-4-yl)- | | | | (3) |
| | | methyl | | | | |
| 70A | 52A (49A) | (2,4-dimethyl-1,3- | 3-(trifluoro- | 32 | 645 | 3.39 |
| | | thiazol-5-yl)- | methyl)phenoxy | | | (2) |
| | | methyl | | | | |
| 71A | 52A (49A) | (2,4-dimethyl-1,3- | 4-(trifluoro- | 31 | 645 | 3.40 |
| | | thiazol-5-yl)- | methyl)phenoxy | | | (3) |
| | | methyl | | | | |
| 72A | 52A (49A) | (2,4-dimethyl-1,3- | 3,4-difluoro- | 53 | 613 | 3.14 |
| | | thiazol-5-yl)- | phenoxy | | | (1) |
| | | methyl | | | | |
| 73A | 52A (49A) | (2,4-dimethyl-1,3- | 3,5-difluoro- | 66 | 613 | 3.18 |
| | | thiazol-5-yl)- | phenoxy | | | (1) |
| | | methyl | | | | |
| 74A | 52A (49A) | (2,4-dimethyl-1,3- | 3-chlorophenoxy | 53 | 611 | 3.22 |
| | | thiazol-5-yl)- | | | | (1) |
| | | methyl | | | | |
| 75A | 52A (49A) | (2,4-dimethyl-1,3- | 3-methylphenoxy | 95 | 591 | 3.33 |
| | | thiazol-5-yl)- | | | | (3) |
| | | methyl | | | | |
| 76A | 55A* (9A) | 2-methoxyethyl | 4-(trifluoro- | 69 | 562 | 3.28 |
| | | | methyl)phenyl | | | (2) |
| 77A | 55A* (9A) | 2-methoxyethyl | 4-(trifluoro- | 77 | 578 | 3.16 |
| | | | methoxy)phenyl | | | (1) |
| 78A | 55A* (9A) | 2-methoxyethyl | 3-(trifluoro- | 58 | 578 | 3.20 |
| | | | methoxy)phenyl | | | (1) |
| 79A | 55A* (9A) | 2-methoxyethyl | 4-fluoro-3-methyl- | 76 | 526 | 2.94 |
| | | | phenyl | | | (1) |
| 80A | 55A (47A) | (2-methyl-1,3- | 3-(trifluoro- | 63 | 615 | 3.42 |
| | | thiazol-4-yl)- | methyl)phenyl | | | (3) |
| | | methyl | | | | |
| 81A | 55A (47A) | (2-methyl-1,3- | 4-(trifluoro- | 38 | 615 | 3.27 |
| | | thiazol-4-yl)- | methyl)phenyl | | | (1) |
| | | methyl | | | | |
| 82A | 55A (39A) | (1-methyl-1H- | 3-(trifluoro- | 65 | 598 | 2.04 |
| | | imidazol-2-yl)- | methyl)phenyl | | | (1) |
| | | methyl | | | | |
| 83A | 55A (39A) | (1-methyl-1H- | 4-(trifluoro- | 58 | 598 | 2.22 |
| | | imidazol-2-yl)- | methyl)phenyl | | | (2) |
| | | methyl | | | | |
| 84A | 55A (49A) | (2,4-dimethyl-1,3- | 4-(trifluoro- | 52 | 629 | 3.40 |
| | | thiazol-5-yl)- | methyl)phenyl | | | (2) |
| | | methyl | | | | |
| 85A | 55A (49A) | (2,4-dimethyl-1,3- | 3-(trifluoro- | 50 | 629 | 3.40 |
| | | thiazol-5-yl)- | methyl)phenyl | | | (2) |
| | | methyl | | | | |
| 86A | 55A (49A) | (2,4-dimethyl-1,3- | 4-methylphenyl | 66 | 575 | 3.00 |
| | | thiazol-5-yl)- | | | | (2) |
| | | methyl | | | | |
| 87A | 57A (56A) | cyclopropyl- | 3-(trifluoro- | 20 | 558 | 3.43 |
| | | methyl | methyl)phenyl | | | (3) |

*Single-mode microwave, 140° C., 1h.

### WORKING EXAMPLES

### Example 1

### 2-({4-[((2-Furylmethyl) {[6(4-methylphenyl)pyrimidin-4-yl]methyl}amino)methyl]phenyl}thio)-2-methylpropanoic acid

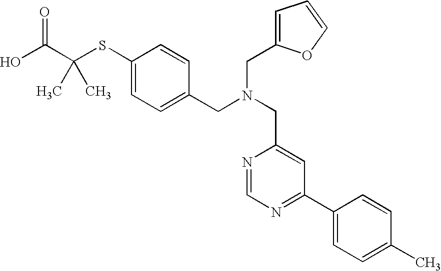

66 mg of the compound from Example 3A (0.12 mmol) are stirred in 5 ml of a 4N solution of hydrogen chloride in dioxane at RT overnight. The solvent is distilled off under reduced pressure and the residue is purified by preparative HPLC (mobile phase: acetonitrile/water with 0.1% formic acid, gradient 20:80→95:5). This gives 20 mg (31% of theory) of the title compound.

LC/MS (method 1): Rₜ=2.52 min; MS (ESIpos): m/z=488 [M+H]⁺.

¹H-NMR (400 MHz, DMSO d₆): δ [ppm]=1.32 (s, 6H), 2.40 (s, 3H), 3.73 (s, 4H), 3.77 (s, 2H), 6.34 (d, 1H), 6.38 (dd, 1H), 7.36-7.42 (m, 6H), 7.60 (d, 1H), 8.01-8.07 (m, 3H), 9.08 (d, 1H), 12.56 (br. s, 1H).

### Example 2

### 2-({4-[((2-Furylmethyl) {[6-(3-trifluoromethylphenyl)pyrimidin-4-yl]methyl)amino}methyl]-phenyl}thio)-2-methylpropanoic acid hydrochloride

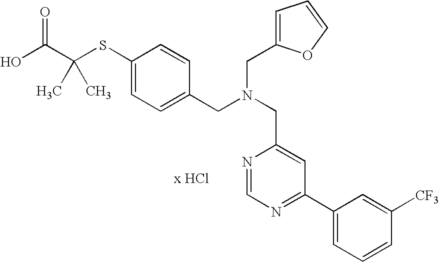

60 mg of the compound from Example 5A (0.12 mmol) are stirred in 5 ml of a 4N solution of hydrogen chloride in dioxane at RT overnight. The solvent is distilled off under reduced pressure. This gives 53 mg (91% of theory) of the title compound.

LC/MS (method 3): Rₜ=2.89 min; MS (ESIpos): m/z=542 [M+H]⁺.

¹H-NMR (400 MHz, DMSO-d₆): δ [ppm]=1.33 (s, 6H), 3.77 (s, 4H), 3.84 (s, 2H), 6.35-6.40 (m, 2H), 7.39 (d, 2H), 7.42 (d, 2H), 7.59 (s, 1H), 7.84 (t, 1H), 7.96 (d, 1H), 8.16 (s, 1H), 8.42-8.47 (m, 2H), 9.18 (s, 1H), 12.57 (br. s, 1H).

### Example 3

### 2-{[4-({(2-Furylmethyl)[6-(3-methylbenzyl)pyrimidin-4-yl]amino}methyl)phenyl]thio}-2-methylpropanoic acid

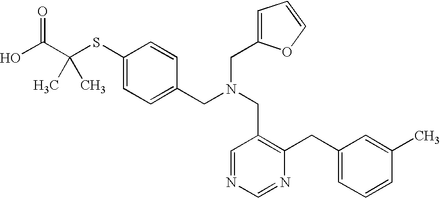

96 mg of the compound from Example 10A (0.18 mmol) are stirred in 5 ml of a 4N solution of hydrogen chloride in dioxane at RT overnight. The solvent is distilled off under reduced pressure and the residue is then purified by preparative HPLC (mobile phase: acetonitrile/water with 0.1% formic acid, gradient 20:80→95:5). This gives 51 mg (55% of theory) of the title compound.

LC/MS (method 2): Rₜ=2.15 min; MS (ESIpos): m/z=488 [M+H]⁺.

¹H-NMR (300 MHz, DMSO-d₆): δ [ppm]=1.35 (s, 6H), 2.77 (s, 3H), 3.78 (s, 2H), 4.78 (br. s, 4H), 6.28 (d, 1H), 6.36 (dd, 1H), 6.69 (br. s, 1H), 6.96-7.05 (m, 3H), 7.10-7.19 (m, 3H), 7.36 (d, 2H), 7.54 (d, 1H), 8.43 (s, 1H), 12.59 (br. s, 1H).

### Example 4

### 2-{[4-({(2-Furylmethyl)[6-(4-methylbenzyl)pyrimidin-4-yl]amino}methyl)phenyl]thio}-2-methylpropanoic acid hydrochloride

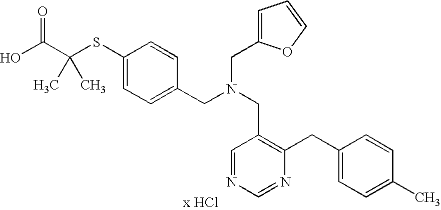

106 mg of the compound from Example 11A (0.18 mmol) are stirred in 5 ml of a 4N solution of hydrogen chloride in dioxane at RT overnight. The solvent is distilled off under reduced pressure and the residue is dried under high vacuum. This gives 96 mg (94% of theory) of the title compound.

LC/MS (method 3): Rₜ=2.21 min; MS (ESIpos): m/z=488 [M+H]⁺.

¹H-NMR (400 MHz, DMSO-d₆): δ [ppm]=1.36 (s, 6H), 2.28 (s, 3H), 3.99 (br. s, 2H), 4.97 (br. s, 4H), 6.40 (s*, 2H), 6.87-7.34 (m, 7H), 7.36 (d, 2H), 7.58 (s, 1H), 8.81 (s, 1H), 12.61 (br. s, 1H).

### Example 5

### 2-({4-[((2-Methoxyethyl) {6-[3-(trifluoromethyl)phenyl]pyrimidin-4-yl}amino)methyl]phenyl}-thio)-2-methylpropanoic acid hydrochloride

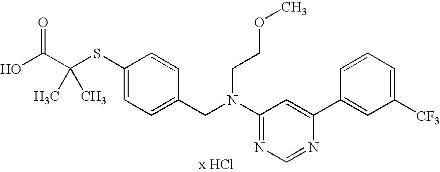

100 mg of the compound from Example 12A (0.18 mmol) are stirred in 5 ml of a 4N solution of hydrogen chloride in dioxane at RT overnight. The solvent is distilled off under reduced pressure and the residue is dried under high vacuum. This gives 94 mg (95% of theory) of the title compound.

LC/MS (method 2): Rₜ=2.61 min; MS (ESIpos): m/z=506 [M+H]⁺.

¹H-NMR (400 MHz, DMSO-d₆): δ [ppm]=1.35 (s, 6H), 3.23 (s, 3H), 3.40-3.61 (m, 2H), 3.83 (br. s, 2H), 4.90 (s, 2H), 7.21-7.45 (br. s, 1H), 7.25 (d, 2H), 7.42 (d, 2H), 7.74 (t, 1H), 7.87 (d, 1H), 8.40 (br. s, 2H), 8.64 (s, 1H), 12.58 (br. s, 1H).

### Example 6

### 2-[(4-{[[6-(3-Chlorophenyl)pyrimidin-4-yl](2-methoxyethyl)amino]methyl}phenyl)thio]-2-methylpropanoic acid hydrochloride

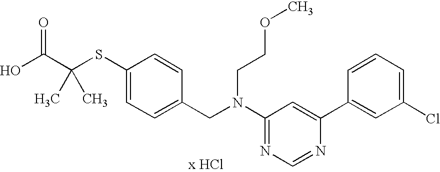

100 mg of the compound from Example 13A (0.18 mmol) are stirred in 5 ml of a 4N solution of hydrogen chloride in dioxane at RT overnight. The solvent is distilled off under reduced pressure and the residue is dried under high vacuum. This gives 96 mg (98% of theory) of the title compound.

LC/MS (method 1): Rₜ=2.34 min; MS (ESIpos): m/z=472 [M+H]⁺.

¹H-NMR (300 MHz, DMSO-d₆): δ [ppm]=1.36 (s, 6H), 3.23 (s, 3H), 3.24-3.84 (m, 2H), 3.92 (br. s, 2H), 5.08 (s, 2H), 7.28 (d, 2H), 7.43 (d, 2H), 7.57-7.52 (m, 2H), 7.85-8.20 (m, 2H), 8.32 (s, 1H), 8.29 (s, 1H).

### Example 7

### 2-[(4-{[[6-(3-Methylphenyl)pyrimidin-4-yl](2-methoxyethyl)amino]methyl}phenyl)thio]-2-methyl-propanoic acid

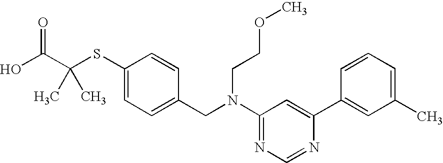

129 mg of the compound from Example 14A (0.18 mmol) are stirred in 5 ml of a 4N solution of hydrogen chloride in dioxane at RT overnight. The solvent is distilled off under reduced pressure and the residue is purified by preparative HPLC (mobile phase: acetonitrile/water with 0.1% formic acid, gradient 20:80→95:5). This gives 100 mg (81% of theory) of the title compound.

LC/MS (method 2): Rₜ=2.09 min; MS (ESIpos): m/z=452 [M+H]⁺.

¹H-NMR (400 MHz, DMSO-d₆): δ [ppm]=1.34 (s, 6H), 2.37 (s, 3H), 3.23 (s, 3H), 3.55 (t, 2H), 3.80 (br. s, 2H), 4.94 (s, 2H), 7.13 (br. s, 1H), 7.24 (d, 2H), 7.28 (m, 1H), 7.36 (t, 1H), 7.41 (d, 2H), 7.76-7.90 (m, 2H), 8.56 (s, 1H), 12.56 (br. s, 1H).

### Example 8

### 2-{[4-({(2-Methoxyethyl)[6-(4-methylphenyl)pyrimidin-4-yl]amino}methyl)phenyl]thio}-2-methylpropanoic acid

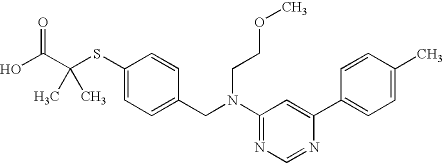

154 mg of the compound from Example 15A (0.30 mmol) are stirred in 5 ml of a 4N solution of hydrogen chloride in dioxane at RT overnight. The solvent is distilled off under reduced pressure and the residue is purified by preparative HPLC (mobile phase: acetonitrile/water with 0.1% formic acid, gradient 20:80→95:5). This gives 60 mg (40% of theory) of the title compound.

LC/MS (method 1): Rₜ=1.87 min; MS (ESIpos): m/z=452 [M+H]⁺.

¹H-NMR (400 MHz, DMSO-d₆): δ [ppm]=1.35 (s, 6H), 2.35 (s, 3H), 3.23 (s, 3H), 3.54 (t, 2H), 3.79 (br. s, 2H), 4.94 (s, 2H), 7.11 (br. s, 1H), 7.24 (d, 2H), 7.27 (d, 2H), 7.41 (d, 2H), 7.95 (m, 2H), 8.55 (s, 1H), 12.57 (br. s 1H).

### Example 9

### 2-({4-[((2-Furylmethyl) {6-[3-(trifluoromethyl)phenyl]pyrimidin-4-yl}amino)methyl]phenyl}thio)-2-methylpropanoic acid hydrochloride

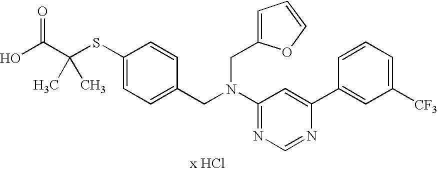

106 mg of the compound from Example 16A (0.18 mmol) are stirred in 5 ml of a 4N solution of hydrogen chloride in dioxane at 50° C. for 3 h. The solvent is distilled off under reduced pressure and the residue is dried under high vacuum. This gives 86 mg (84% of theory) of the title compound.

LC/MS (method 2): Rₜ=2.88 min; MS (ESIpos): m/z=528 [M+H]⁺.

¹H-NMR (400 MHz, DMSO-d₆): δ [ppm]=1.35 (s, 6H), 4.88-5.01 (m, 4H), 6.37-6.42 (m, 2H), 7.24 (d, 2H), 7.27-7.87 (m, 1H), 7.39 (d, 2H), 7.58 (s, 1H), 7.77 (t, 1H), 7.90 (d, 1H), 8.40 (br. s, 2H), 8.73 (s, 1H), 12.60 (br. s, 1H).

### Example 10

### 2-({4-[((2-Furylmethyl) {6-[3-(chloromethyl)phenyl]pyrimidin-4-yl}amino)methyl]phenyl}thio)-2-methylpropanoic acid hydrochloride

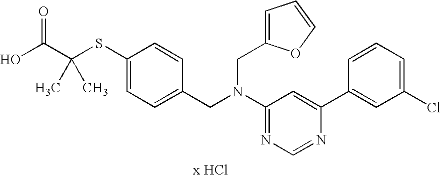

80 mg of the compound from Example 17A (0.15 mmol) are stirred in 5 ml of a 4N solution of hydrogen chloride in dioxane at RT overnight. The solvent is distilled off under reduced pressure and the residue is dried under high vacuum. This gives 64 mg (81% of theory) of the title compound.

LC/MS (method 1): Rₜ=2.57 min; MS (ESIpos): m/z=494 [M+H]⁺.

¹H-NMR (400 MHz, DMSO-d₆): δ [ppm]=1.35 (s, 6H), 4.89 (br. s, 2H), 4.92 (s, 2H), 6.38 (s*, 2H), 7.11-7.56 (m, 1H), 7.22 (d, 2H), 7.39 (d, 2H), 7.49-7.60 (m, 3H), 8.05 (br. s, 1H), 8.16 (br. s, 1H), 8.63 (s, 1H), 12.58 (br. s, 1H).

### Example 11

### 2-Methyl-2-{[4-({[6-(3-methylphenyl)pyrimidin-4-yl]amino}methyl)phenyl]thio}propanoic acid hydrochloride

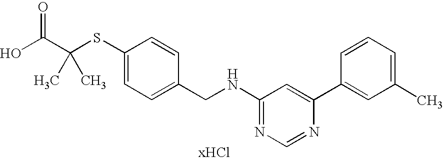

150 mg of the compound from Example 18A (0.33 mmol) are stirred in 5 ml of a 4N solution of hydrogen chloride in dioxane at RT overnight. The solvent is distilled off under reduced pressure and the residue is dried under high vacuum. This gives 140 mg (88% of theory) of the title compound.

LC/MS (method 3): Rₜ=1.89 min; MS (ESIpos): m/z=394 [M+H]⁺.

¹H-NMR (400 MHz, DMSO-d₆): δ [ppm]=1.36 (s, 6H), 2.41 (s, 3H), 4.76 (d, 2H), 7.08 (br. s, 1H), 7.36 (d, 2H), 7.41-7.54 (m, 4H), 7.64-7.65 (m, 2H), 8.77 (br. s, 1H), 9.48 (br. s, 1H), 12.60 (br. s, 1H).

### Example 12

### 2-[(4-{[[6-(4-Fluoro-3-methylphenyl)pyrimidin-4-yl](2-furylmethyl)amino]methyl}phenyl)thio]-2-methylpropanoic acid

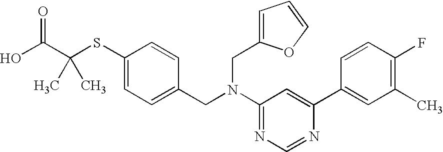

67 mg of the compound from Example 19A (0.12 mmol) are stirred in 5 ml of a 4N solution of hydrogen chloride in dioxane at RT overnight. The solvent is distilled off under reduced pressure and the crude material is purified by preparative HPLC (mobile phase: acetonitrile/water with 0.1% formic acid, gradient 20:80→95:5). This gives 18 mg (28% of theory) of the title compound.

LC/MS (method 1): Rₜ=2.39 min; MS (ESIpos): m/z=492 [M+H]⁺.

¹H-NMR (400 MHz, DMSO-d₆): δ [ppm]=1.35 (s, 6H), 2.30 (s, 3H), 4.88 (br. s, 2H), 4.91 (s, 2H), 6.38 (s*, 2H), 7.19-7.45 (m, 4H), 7.39 (d, 2H), 7.58 (s, 1H), 7.94 (br. s, 1H), 8.03 (br. s, 1H), 8.61 (s, 1H), 12.58 (br. s, 1H).

The working examples 13-16 listed in Table 3 below are obtained from the corresponding starting materials (Examples 20A-23A) analogously to the examples described above:

TABLE 3

| | | | | Rₜ |
|---|---|---|---|---|
| | | Yield | | [min] |
| Example | | [% of | MS: m/z | (LC/MS |
| No. | Structure | theory] | [M + H]⁺ | method) |
| 13 | 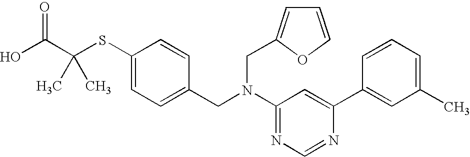 | 96 | 474 | 2.40(2) |
| 14 | 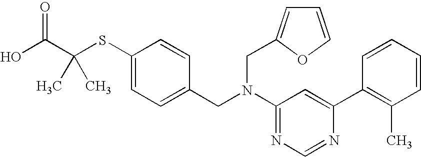 | 71 | 474 | 2.24(3) |
| 15 | 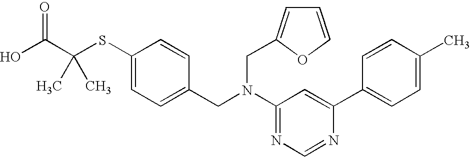 | 87 | 474 | 2.37(2) |
| 16 | 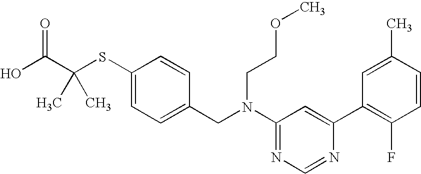 | 89 | 470 | 2.11(1) |

### Example 17

### 2-({4-[((2-Methoxyethyl) {2-[3-(trifluoromethyl)phenyl]pyrimidin-4-yl}amino)methyl]phenyl}-thio)-2-methylpropanoic acid hydrochloride

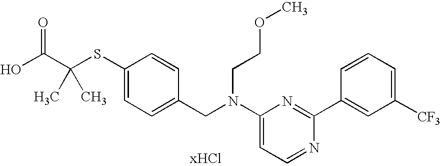

110 mg of the compound from Example 25A (0.20 mmol) are stirred in 5 ml of a 4N solution of hydrogen chloride in dioxane at RT overnight. The solvent is distilled off under reduced pressure and the residue is dried under high vacuum. This gives 100 mg (90% of theory) of the title compound.

LC/MS (method 1): Rₜ=2.43 min; MS (ESIpos): m/z=506 [M+H]⁺.

¹H-NMR (400 MHz, DMSO-d₆): δ [ppm]=1.34 (s, 6H), 3.25 (s, 3H), 3.69 (m, 2H), 3.84 and 4.07 (2 br. s, 2H), 4.95 and 5.10 (2 br. s, 2H), 6.82 and 7.10 (2 br. s, 1H), 7.31 (d, 2H), 7.42 (d, 2H), 7.79 (br. s, 1H), 7.96 (br. s, 1H), 8.24-8.68 (m, 3H).

### Example 18

### 2-({4-[((2-Methoxyethyl) {2-[3-methylphenyl]pyrimidin-4-yl}amino)methyl]phenyl}thio)-2-methylpropanoic acid hydrochloride

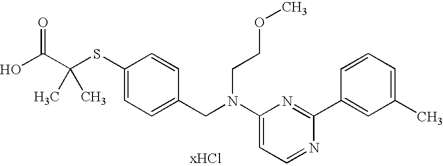

81 mg of the compound from Example 26A (0.19 mmol) are stirred in 5 ml of a 4N solution of hydrogen chloride in dioxane at RT overnight. The solvent is distilled off under reduced pressure and the residue is dried under high vacuum. This gives 81 mg (86% of theory) of the title compound.

LC/MS (method 3): Rₜ=2.04 min; MS (ESIpos): m/z=452 [M+H]⁺.

¹H-NMR (400 MHz, DMSO-d₆): δ [ppm]=1.35 (br. s, 6H), 2.38 (br. s, 3H), 3.25 (s, 3H), 3.61 (m, 2H), 3.85 and 4.10 (2 br. s, 2H), 5.00 and 5.14 (2 br. s, 2H), 6.89 and 7.15 (2 br. s, 1H), 7.32 (br. s, 2H), 7.39-7.55 (m, 4H), 7.92 (br. s, 1H), 8.11 (br. s, 1H), 8.34 (s, 1H).

### Example 19

### 2({4-[((2-Methoxyethyl) {2-[3-chlorophenyl]pyrimidin-4-yl}amino)methyl]phenyl}thio)-2-methylpropanoic acid hydrochloride

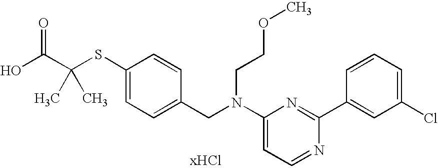

78 mg of the compound from Example 27A (0.19 mmol) are stirred in 5 ml of a 4N solution of hydrogen chloride in dioxane at RT overnight. The solvent is distilled off under reduced pressure and the residue is dried under high vacuum. This gives 64 mg (85% of theory) of the title compound.

LC/MS (method 1): Rₜ=2.25 min; MS (ESIpos): m/z=472 [M+H]⁺.

¹H-NMR (400 MHz, DMSO-d₆): δ [ppm]=1.35 (s, 6H), 3.25 (s, 3H), 3.59 (br. s, 2H), 3.75 and 4.00 (2 br. s, 2H), 4.78-5.10 (m, 2H), 6.68 and 6.94 (2 br. s, 1H), 7.28 (d, 2H), 7.42 (d, 2H), 7.53 (br. s, 1H), 7.59 (m, 1H), 8.04-8.42 (m, 3H), 12.58 (br. s, 1H).

### Example 20

### 2-[(4-{[(6-{[4-Fluoro-3-(trifluoromethyl)phenyl]amino}pyrimidin-4-yl)(2-furylmethyl)amino]-methyl}phenyl)thio]-2-methylpropanoic acid

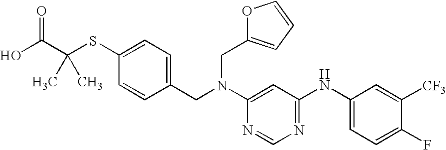

Triethylamine (40 μl) and 4-fluoro-3-(trifluoromethyl)aniline (36 mg, 0.2 mmol) are added to the compound from Example 6A (47 mg, 0.1 mmol) in DMF (800111). The mixture is heated at 100° C. for 16 h and the solution is then filtered and evaporated to dryness. Trifluoroacetic acid (200 μl) is added, and the mixture is stirred at room temperature for 5 h. DMF is added and the mixture is purified directly by preparative HPLC. This gives 2.3 mg (4% of theory) of the title compound.

LC/MS (method 4): Rₜ=2.13 min; MS (ESIpos): m/z=562 [M+H]⁺.

¹H-NMR (400 MHz, DMSO-d₆): δ [ppm]=1.32 (s, 6H), 4.6 (br. m, 4H), 5.9 (s, 1H), 6.3 (d, 1H), 6.4 (d, 1H), 7.2 (d, 2H), 7.4 (m, 3H), 7.60 (d, 1H), 7.8 (m, 1H), 8.1 (m, 1H), 8.3 (s, 1H), 9.4 (s, 1H), 12.6 (br. s, 1H).

### Example 21

### 2-[(4-{[{6-[(3-Chloro-4-fluorophenyl)amino]pyrimidin-4-yl}(2-furylmethyl)amino]methyl}-phenyl)thio]-2-methylpropanoic acid

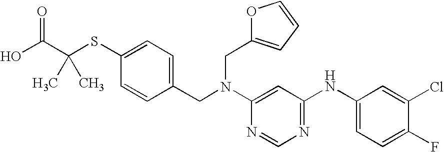

Triethylamine (40 μl) and 4-fluoro-3-chloroaniline (36 mg, 0.2 mmol) are added to the compound from Example 6A (47 mg, 0.1 mmol) in DMF (800 μl). The mixture is heated at 100° C. for 16 h, and the solution is then filtered and evaporated to dryness. Trifluoroacetic acid (200 111) is added, and the mixture is stirred at room temperature for 5 h. DMF is added and the mixture is purified directly by preparative HPLC. This gives 3.1 mg (5% of theory) of the title compound.

LC/MS (method 4): Rₜ=2.27 min; MS (ESIpos): m/z=528 [M+H]⁺.

¹H-NMR (400 MHz, DMSO-d₆): δ [ppm]=1.32 (s, 6H), 4.6 (br. m, 4H), 5.9 (s, 1H), 6.3 (d, 1H), 6.4 (d, 1H), 7.2-7.4 (m, 6H), 7.6 (s, 1H), 7.9 (m, 1H), 8.3 (s, 1H), 9.3 (s, 1H).

### Example 22

### 2-{[4({(2-Furylmethyl)[6-(4-methylphenoxy)pyrimidin-4-yl]amino}methyl)phenyl]thio}-2-methylpropanoic acid hydrochloride

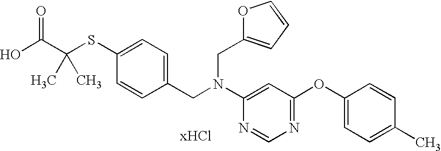

50 mg of the compound from Example 28A (0.1 mmol) are stirred in 5 ml of a 4N solution of hydrogen chloride in dioxane at RT overnight. The solvent is distilled off under reduced pressure and the residue is dried under high vacuum. This gives 48 mg (88% of theory) of the title compound.

LC/MS (method 3): Rₜ=2.84 min; MS (ESIpos): m/z=490 [M+H]⁺.

¹H-NMR (400 MHz, DMSO): δ [ppm]=1.36 (s, 6H), 2.31 (s, 3H), 4.80 (br. s, 4H), 6.14 (br. s, 1H), 6.31 (d, 1H), 6.38 (dd, 1H), 6.97 (d, 2H), 7.13-7.22 (m, 4H), 7.38 (d, 2H), 7.58 (dd, 1H), 8.23 (s, 1H).

### Example 23

### 2-{[4-{(2-Furylmethyl)[6-phenoxypyrimidin-4-yl]amino}methyl)phenyl]thio}-2-methylpropanoic acid hydrochloride

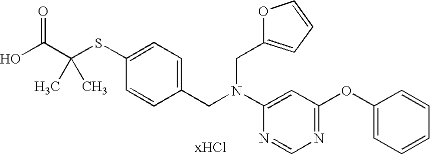

30 mg of the compound from Example 29A (0.1 mmol) are stirred in 5 ml of a 4N solution of hydrogen chloride in dioxane at RT overnight. The solvent is distilled off under reduced pressure and the residue is dried under high vacuum. This gives 35 mg of the title compound in a purity of 80% (85% of theory).

LC/MS (method 3): Rₜ=2.73 min; MS (ESIpos): m/z=476 [M+H]⁺.

¹H-NMR (400 MHz, DMSO-d₆): δ [ppm]=1.36 (s, 6H), 4.65-4.90 (br. m, 4H), 6.18 (br. m, 1H), 6.31 (d, 1H), 6.38 (dd, 1H), 7.10 (d, 2H), 7.14-7.28 (m, 3H), 7.35-7.42 (m, 4H), 7.58 (d, 1H), 8.24 (s, 1H).

### Example 24

### 2-[(4-{[[6-(3-Chlorophenyl)pyrimidin-4-yl](prop-2-yn-1-yl)amino]methyl}phenyl)thio]-2-methylpropanoic acid

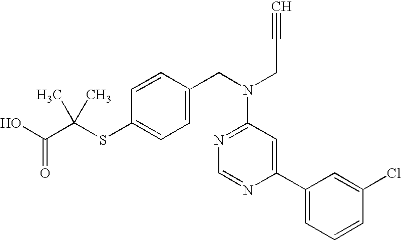

62 mg of the compound from Example 32A (0.12 mmol) are initially charged in 3 ml of dichloromethane, and 3 ml of trifluoroacetic acid are then added with ice cooling. After one hour of stirring, the solvent is distilled off under reduced pressure and the residue is taken up in saturated sodium bicarbonate solution and extracted twice with dichloromethane. The organic phases are combined and dried with sodium sulfate, the solvent is removed under reduced pressure and the residue is dried under high vacuum. This gives 50 mg (91% of theory) of the title compound.

LC/MS (method 2): Rₜ=2.67 min; MS (ESIpos): m/z=452 [M+H]⁺.

¹H-NMR (400 MHz, DMSO-d₆): δ [ppm]=1.35 (s, 6H), 3.22 (t, 1H), 4.49 (d, 2H), 4.97 (s, 2H), 7.27-7.37 (m, 3H), 7.42 (d, 2H), 7.50-7.60 (m, 2H), 8.07 (d, 1H), 8.17 (s, 1H), 8.67 (s, 1H), 12.59 (br. s, 1H).

### Example 25

### 2-Methyl-2-({4-[((1,3-thiazol-2-ylmethyl) {6-[3-(trifluoromethyl)phenyl]pyrimidin-4-yl}amino)methyl]phenyl}thio)propanoic acid

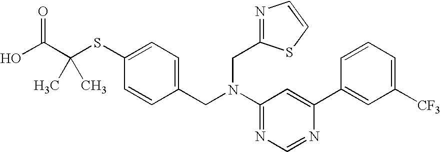

130 mg of the compound from Example 37A (0.216 mmol) are initially charged in 2 ml of dichloromethane, and 1 ml of TFA is added. The mixture is stirred at RT for 1 h and then concentrated using a rotary evaporator. The residue is taken up in ethyl acetate and washed first with 20% strength sodium acetate solution and then with saturated sodium chloride solution. The mixture is then dried with sodium sulfate, and the solvent is removed under reduced pressure. This gives 101.4 mg of the title compound (86% of theory).

LC/MS (method 1): Rₜ=2.59 min; MS (ESIpos): m/z=545 [M+H]⁺.

¹H-NMR (400 MHz, DMSO-d₆): δ [ppm]=1.35 (s, 6H), 5.04 (s, 2H), 5.21 (s, 2H), 7.31 (d, 2H), 7.42 (d, 2H), 7.52 (br. s, 1H), 7.65 (d, 1H), 7.71-7.79 (m, 2H), 7.87 (d, 1H), 8.38 (d, 1H), 8.41 (s, 1H), 8.73 (s, 1H).

### Example 26

### 2-Methyl-2-[(4-{[[(1-methyl-1H-imidazol-2-yl)methyl](6-{[4-(trifluoromethyl)phenyl]amino}-pyrimidin-4-yl)amino]methyl}phenyl)thio]propanoic acid

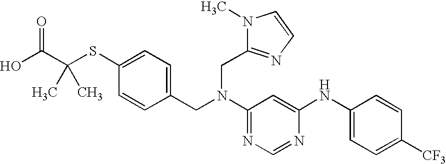

50 mg of the compound from Example 40A (0.082 mmol) are initially charged in 2 ml of dichloromethane, and 1 ml of TFA is added. The mixture is stirred at RT for 1 h and then concentrated using a rotary evaporator. The residue is taken up in ethyl acetate and washed first with 20% strength sodium acetate solution and then with saturated sodium chloride solution. The mixture is then dried with sodium sulfate, and the solvent is removed under reduced pressure. This gives 40 mg of the title compound (88% of theory).

LC/MS (method 1): Rₜ=1.77 min; MS (ESIpos): m/z=557 [M+H]⁺.

¹H-NMR (400 MHz, DMSO-d₆): δ [ppm]=1.37 (s, 6H), 3.65 (s, 3H), 4.77 (s, 2H), 4.93 (s, 2H), 5.98 (s, 1H), 6.95 (s*, 1H), 7.07-7.30 (m, 3H), 7.41 (d, 2H), 7.57 (d, 2H), 7.76 (d, 2H), 8.31 (s, 1H), 9.52 (s, 1H).

### Example 27

### 2-[(4-{[(2-Methoxyethyl)(6-{[3-(trifluoromethyl)phenyl]amino}pyrimidin-4-yl)amino]methyl}-phenyl)thio]-2-methylpropanoic acid

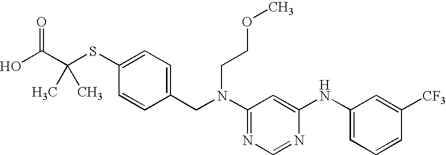

68 mg of the compound from Example 41A (0.118 mmol) are initially charged in 3 ml of dichloromethane, and 3 ml of TFA are added. The mixture is stirred at RT for 1 h and then concentrated using a rotary evaporator. The residue is taken up in saturated sodium bicarbonate solution and then extracted twice with ethyl acetate. The combined organic phases are dried with sodium sulfate, and the solvent is removed under reduced pressure. This gives 50 mg of the title compound (81% of theory).

LC/MS (method 1): Rₜ=2.30 min; MS (ESIpos): m/z=521 [M+H]⁺.

¹H-NMR (400 MHz, DMSO-d₆): δ [ppm]=1.36 (s, 6H), 3.23 (s, 3H), 3.50 (t, 2H), 3.66 (br. s, 2H), 4.79 (s, 2H), 5.88 (s, 1H), 7.17-7.26 (m, 3H), 7.41 (d, 2H), 7.47 (d, 1H), 7.75 (d, 1H), 8.13 (s, 1H), 8.26 (s, 1H), 9.41 (s, 1H), 12.59 (br. s, 1H).

### Example 28

### 2-[(4-{[{6-[4-(4-Fluorophenyl)piperazin-1-yl]pyrimidin-4-yl}(2-methoxyethyl)amino]methyl}-phenyl)thio]-2-methylpropanoic acid

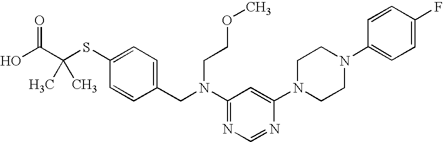

84 mg of the compound from Example 51A (0.141 mmol) are initially charged in 2 ml of dichloromethane, and 1 ml of TFA is added. The mixture is stirred at RT for 1 h and then concentrated using a rotary evaporator. The residue is taken up in ethyl acetate and washed first with 20% strength sodium acetate solution and then with saturated sodium chloride solution. The mixture is then dried with sodium sulfate, and the solvent is removed under reduced pressure. This gives 70 mg of the title compound (90% of theory).

LC/MS (method 3): Rₜ=2.20 min; MS (ESIpos): m/z=540 [M+H]⁺.

¹H-NMR (400 MHz, DMSO-d₆): δ [ppm]=1.34 (s, 6H), 3.09 (m, 4H), 3.22 (s, 3H), 3.49 (t, 2H), 3.56-3.72 (m, 6H), 4.81 (s, 2H), 5.80 (s, 1H), 6.94-7.11 (m, 4H), 7.19 (d, 2H), 7.39 (d, 2H), 8.09 (s, 1H).

### Example 29

### 2-({4-[((2-Methoxyethyl) {6-[(trans-4-methylcyclohexyl)oxy]pyrimidin-4-yl}amino)methyl]-phenyl}thio)-2-methylpropanoic acid

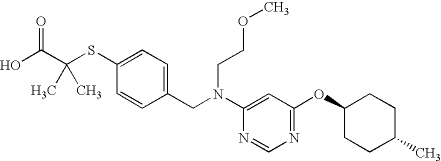

150 mg of the compound from Example 9A (0.332 mmol) and 45.5 mg of trans-4-methylcyclohexanol (0.398 mmol) are dissolved in 2 ml of DMSO. 74.5 mg of potassium tert-butoxide (0.664 mmol) are then added. The reaction mixture is stirred at RT overnight, then neutralized with 1 N hydrochloric acid and extracted twice with ethyl acetate. The combined organic phases are dried with sodium sulfate and the solvent is removed under reduced pressure. The residue is purified by preparative HPLC (mobile phase: acetonitrile/water with 0.1% formic acid, gradient 20:80→95:5). In addition to the corresponding tert-butyl ester (19.2 mg, 11% of theory), 32 mg of the title compound (20% of theory) are isolated.

LC/MS (method 3): Rₜ=2.96 min; MS (ESIpos): m/z=474 [M+H]⁺.

¹H-NMR (400 MHz, DMSO-d₆): δ [ppm]=0.87 (d, 3H), 1.08 (dq, 2H), 1.25-1.38 (m, 3H), 1.35 (s, 6H), 1.69 (d, 2H), 1.98 (d, 2H), 3.21 (s, 3H), 3.47 (t, 2H), 3.66 (br. s, 2H), 4.78 (br. s, 2H), 4.83 (m, 1H), 5.80 (br. s, 1H), 7.18 (d, 2H), 7.39 (d, 2H), 8.21 (s, 1H).

### Example 30

### 2-{[4-({[6-(Cyclohexyloxy)pyrimidin-4-yl][(3,5-dimethylisoxazol-4-yl)methyl]amino}methyl)phenyl]thio}-2-methylpropanoic acid

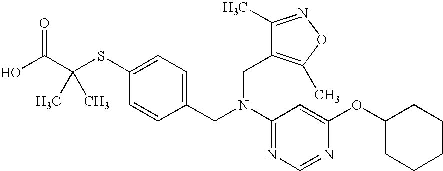

Analogously to the preparation of Example 25, 63.0 mg of the compound from Example 45A (0.111 mmol) give 56 mg of the title compound (99% of theory).

LC/MS (method 1): Rₜ=2.67 min; MS (ESIpos): m/z=511 [M+H]⁺.

¹H-NMR (400 MHz, DMSO-d₆): δ [ppm]=1.15-1.44 (m, 5H), 1.35 (s, 6H), 1.52 (m, 1H), 1.69 (m, 2H), 1.88 (m, 2H), 2.06 (s, 3H), 2.19 (s, 3H), 4.60 (s, 2H), 4.68 (s, 2H), 4.93 (m, 1H), 5.93 (s, 1H), 7.12 (d, 2H), 7.38 (d, 2H), 8.29 (s, 1H).

### Example 31

### 2-{[4-({{6-[(trans-4-Methoxycyclohexyl)oxy]pyrimidin-4-yl}[(2-methyl-1,3-thiazol-4-yl)methyl]amino}methyl)phenyl]thio}-2-methylpropanoic acid

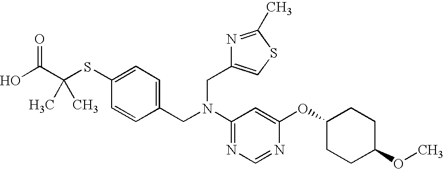

233 mg of potassium tert-butoxide (2.08 mmol) are added to 270 mg of trans-4-methoxycyclohexanol (2.08 mmol; obtained from the cis/trans mixture via the monophthalate: D. S. Noyce, G. L. Woo, B. R. Thomas, J. Org. Chem. 25 (1960), 260-262), and the mixture is stirred at RT for 15 min. 700 mg of the compound from Example 47A (1.39 mmol) are then added, and the reaction mixture is then stirred at RT overnight. The mixture is taken up in water, neutralized with 1 N hydrochloric acid and extracted twice with ethyl acetate. The combined organic phases are dried with sodium sulfate, and the solvent is removed using a rotary evaporator. Without further workup, the residue is directly taken up in 10 ml of dichloromethane, and 5 ml of TFA are added. After 2 h of stirring at RT, the reaction mixture is concentrated and the residue is taken up in ethyl acetate. The mixture is washed with 20% strength sodium acetate solution and with concentrated sodium chloride solution. The solvent is removed under reduced pressure and the residue is purified by preparative HPLC (mobile phase: acetonitrile/water with 0.1% formic acid, gradient 20:80→95:5). In addition to the corresponding tert-butyl ester, 160 mg (22% of theory) of the title compound are obtained.

LC/MS (method 3): Rₜ=2.53 min; MS (ESIpos): m/z=543 [M+H]⁺.

¹H-NMR (400 MHz, DMSO-d₆): δ [ppm]=1.22-1.46 (m, 4H), 1.36 (s, 6H), 1.94 (m, 4H), 2.61 (s, 3H), 3.18 (m, 1H), 3.22 (s, 3H), 4.72 (br. s, 2H), 4.79-5.01 (m, 3H), 5.91 (br. s, 1H), 7.18-7.24 (m, 3H), 7.39 (d, 2H), 8.24 (s, 1H), 12.60 (br. s, 1H).

### Example 32

### 2-{[4-({[6(Cyclohexyloxy)pyrimidin-4-yl][(2,4-dimethyl-1,3-thiazol-5-yl)methyl]amino}methyl)phenyl]thio}-2-methylpropanoic acid

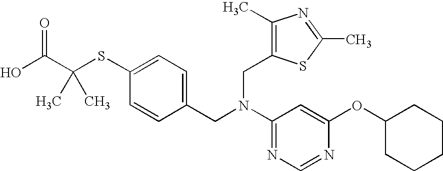

Analogously to the preparation of Example 25, 48.0 mg of the compound from Example 50A (0.111 mmol) give 34 mg of the title compound (74% of theory).

LC/MS (method 2): Rₜ=2.85 min; MS (ESIpos): m/z=527 [M+H]⁺.

¹H-NMR (400 MHz, DMSO-d₆): δ [ppm]=1.19-1.43 (m, 5H), 1.35 (s, 6H), 1.52 (m, 1H), 1.68 (m, 2H), 1.87 (m, 2H), 2.21 (s, 3H), 4.68 (br. s, 2H), 4.84 (br. s, 2H), 4.92 (m, 1H), 5.85 (s, 1H), 7.18 (d, 2H), 7.39 (d, 2H), 8.30 (s, 1H), 12.59 (br. s, 1H).

### Example 33

### 2-Methyl-2-({4-[([(2-methyl-1,3-thiazol-4-yl)methyl]{6-[3-(trifluoromethyl)phenoxy]pyrimidin-4-yl}amino)methyl]phenyl}thio)propanoic acid

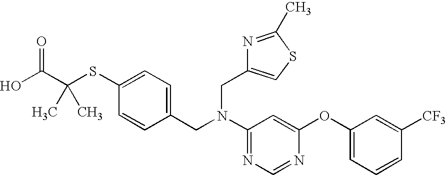

Analogously to the preparation of Example 25, 400 mg of the compound from Example 52A (0.634 mmol) give 277 mg of the title compound (76% of theory).

LC/MS (method 3): Rₜ=2.89 min; MS (ESIpos): m/z=575 [M+H]⁺.

¹H-NMR (400 MHz, DMSO-d₆): δ [ppm]=1.36 (s, 6H), 2.62 (s, 3H), 4.74 (br. s, 2H), 4.94 (br. s, 2H), 6.32 (br. s, 1H), 7.21-7.28 (m, 3H), 7.40 (d, 2H), 7.45 (d, 1H), 7.53 (s, 1H), 7.57-7.62 (m, 2H), 8.24 (s, 1H), 12.60 (br. s, 1H).

### Example 34

### 2-({4-[([(3,5-Dimethylisoxazol-4-yl)methyl]{6-[3-(trifluoromethyl)phenoxy]pyrimidin-4-yl}-amino)methyl]phenyl}thio)-2-methylpropanoic acid

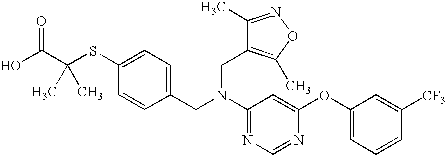

Analogously to the preparation of Example 25, 80 mg of the compound from Example 53A (0.127 mmol) give 59 mg of the title compound (77% of theory).

LC/MS (method 1): Rₜ=2.64 min; MS (ESIpos): m/z=573 [M+H]⁺.

¹H-NMR (400 MHz, DMSO-d₆): δ [ppm]=1.36 (s, 6H), 2.09 (s, 3H), 2.21 (s, 3H), 4.65 (s, 2H), 4.76 (s, 2H), 6.23 (s, 1H), 7.16 (d, 2H), 7.40 (d, 2H), 7.45 (d, 1H), 7.54 (s, 1H), 7.57-7.68 (m, 2H), 8.29 (s, 1H), 12.61 (br. s, 1H).

### Example 35

### 2-{[4-({(2-Methoxyethyl)[6-(4-methylphenoxy)pyrimidin-4-yl]amino}methyl)phenyl]thio}-2-methylpropanoic acid

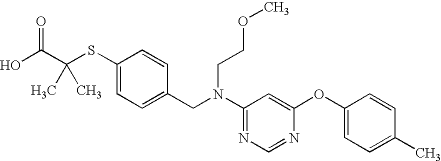

Analogously to the preparation of Example 25, 950 mg of the compound from Example 54A (1.81 mmol) give 590 mg of the title compound (70% of theory).

LC/MS (method 3): Rₜ=2.66 min; MS (ESIpos): m/z=468 [M+H]⁺.

¹H-NMR (400 MHz, DMSO-d₆): δ [ppm]=1.36 (s, 6H), 2.30 (s, 3H), 3.21 (s, 3H), 3.49 (t, 2H), 3.69 (br. s, 2H), 4.80 (br. s, 2H), 6.02 (br. s, 1H), 6.96 (d, 2H), 7.18 (d*, 4H), 7.40 (d, 2H), 8.18 (s, 1H), 12.60 (br. s, 1H).

### Example 36

### 2-({4-[((2-Methoxyethyl) {6-[3-(trifluoromethyl)phenyl]pyrimidin-4-yl}amino)methyl]phenyl}-thio)-2-methylpropanoic acid

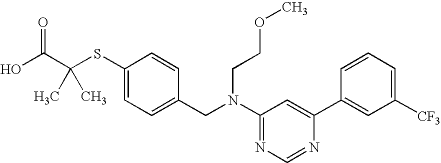

7.12 g of the compound from Example 12A (12.677 mmol) are taken up in 30 ml of dichloromethane and cooled in an ice bath, and 30 ml of TFA are added. The reaction mixture is stirred at RT for 1 h. The highly volatile components are then removed using a rotary evaporator. Saturated sodium bicarbonate solution is added to the residue, and the mixture is extracted twice with ethyl acetate. The combined organic phases are washed successively with water, 20% strength sodium acetate solution and concentrated sodium chloride solution and then dried over sodium sulfate. The solvent is distilled off under reduced pressure and the residue is dried under high vacuum. This gives 5.80 g (91% of theory) of the title compound in a purity of 97% (LC/MS). By recrystallization from ethanol (at a concentration of about 30 mg/ml), the product can be purified to a purity of >99%. Here, 4.10 g of the title compound are recovered (47% of theory).

LC/MS (method 3): Rₜ=2.64 min; MS (ESIpos): m/z=506 [M+H]⁺.

¹H-NMR (300 MHz, DMSO-d₆): δ [ppm]=1.35 (s, 6H), 3.23 (s, 3H), 3.55 (t, 2H), 3.82 (br. s*, 2H), 4.97 (s, 2H), 7.15-7.47 (br. s, 1H), 7.25 (d, 2H), 7.41 (d, 2H), 7.73 (t, 1H), 7.85 (d, 1H), 8.41 (br. s, 2H), 8.62 (s, 1H), 12.58 (br. s, 1H).

### Example 37

### 2-({4-[([(3,5-Dimethylisoxazol-4-yl)methyl]{6-[3-(trifluoromethyl)phenyl]pyrimidin-4-yl}amino)methyl]phenyl}thio)-2-methylpropanoic acid

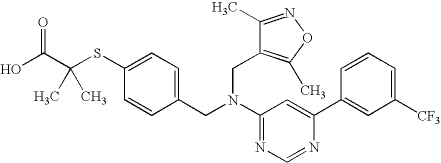

Analogously to the preparation of Example 25, 44 mg of the compound from Example 55A (0.072 mmol) give 33 mg of the title compound (77% of theory).

LC/MS (method 1): Rₜ=2.58 min; MS (ESIpos): m/z=557 [M+H]⁺.

¹H-NMR (400 MHz, DMSO-d₆): δ [ppm]=1.34 (s, 6H), 2.08 (s, 3H), 2.23 (s, 3H), 4.74 (s, 2H), 4.88 (s, 2H), 7.18 (d, 2H), 7.40 (d, 2H), 7.48 (s, 1H), 7.74 (t, 1H), 7.87 (d, 1H), 8.35-8.46 (m, 2H), 8.72 (s, 1H), 12.58 (br. s, 1H).

### Example 38

### 2-({4-[((2-Fluoroethyl) {6-[3-(trifluoromethyl)phenyl]pyrimidin-4-yl} amino)methyl]phenyl}thio)-2-methylpropanoic acid

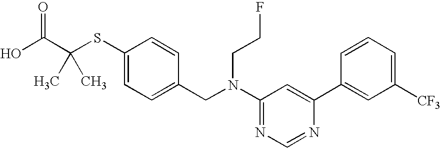

Analogously to the preparation of Example 25, 87 mg of the compound from Example 57A (0.158 mmol) give 65 mg of the title compound (82% of theory).

LC/MS (method 1): Rₜ=2.49 min; MS (ESIpos): m/z=494 [M+H]⁺.

¹H-NMR (300 MHz, DMSO-d₆): δ [ppm]=1.35 (s, 6H), 3.91-4.07 (m, 2H), 4.68 (dt, 2H), 5.00 (s, 2H), 7.27 (d, 2H), 7.38 (br. s, 1H), 7.42 (d, 2H), 7.74 (t, 1H), 7.86 (d, 1H), 8.41 (br. s, 2H), 8.64 (s, 1H), 12.33 (br. s, 1H).

The working examples 39-73 of the general formula (B) listed in Table 4 below are obtained analogously to the examples described above:

TABLE 4

| | |
|---|---|
| | (B) |
| 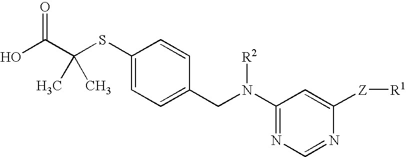 | |

| | Synthesis | | | | | |
|---|---|---|---|---|---|---|
| | analogously | | | | | Rₜ |
| | to Example | | | Yield | MS: | [min] |
| Example | No. (from | | | [% of | m/z | (LC/MS |
| No. | Example No.) | R²— | R¹—Z— | theory] | [M + H]⁺ | method) |
| 39 | 45A, then 25 | 2-methoxyethyl | trans-4-methoxy- | 34 | 490 | 2.23 |
| | (9A) | | cyclohexyloxy | | | (1) |
| 40 | 45A* (9A) | 2-methoxyethyl | cyclohexyloxy | 14 | 460 | 2.70 |
| | | | | | | (2) |
| 41 | 45A* (9A) | 2-methoxyethyl | 4-trifluoromethyl- | 9 | 528 | 2.68 |
| | | | cyclohexyloxy | | | (1) |
| 42 | 25 (59A) | (3,5-dimethyl- | trans-4-methoxy- | 87 | 541 | 2.35 |
| | | isoxazol-4-yl)- | cyclohexyloxy | | | (1) |
| | | methyl | | | | |
| 43 | 45A* (9A) | 2-methoxyethyl | 4-ethylcyclo- | 17 | 488 | 2.93 |
| | | | hexyloxy | | | (1) |
| 44 | 45A* (9A) | 2-methoxyethyl | 4-propylcyclo- | 15 | 502 | 3.09 |
| | | | hexyloxy | | | (1) |
| 45 | 27 (60A) | (3,5-dimethyl- | trans-4-methyl- | 23 | 525 | 3.07 |
| | | isoxazol-4-yl)- | cyclohexyloxy | | | (3) |
| | | methyl | | | | |
| 46 | 45A* (47A) | (2-methyl-1,3- | tetrahydro-2H- | 11 | 515 | 2.38 |
| | | thiazol-4-yl)- | pyran-4-yloxy | | | (3) |
| | | methyl | | | | |
| 47 | 25 (61A) | (2,4-dimethyl- | trans-4-methyl- | 70 | 541 | 3.07 |
| | | 1,3-thiazol-5-yl)- | cyclohexyloxy | | | (3) |
| | | methyl | | | | |
| 48 | 25 (62A) | (2,4-dimethyl- | trans-4-methoxy- | 96 | 557 | 2.33 |
| | | 1,3-thiazol-5-yl)- | cyclohexyloxy | | | (1) |
| | | methyl | | | | |
| 49 | 27 (63A) | 2-methoxyethyl | 4-(trifluoro- | 85 | 522 | 2.79 |
| | | | methyl)phenoxy | | | (2) |
| 50 | 27 (64A) | (2-methyl-1,3- | 4-(trifluoro- | 85 | 575 | 2.84 |
| | | thiazol-4-yl)- | methyl)phenoxy | | | (2) |
| | | methyl | | | | |
| 51 | 25 (65A) | (1,3-thiazol-2- | 4-methylphenoxy | 73 | 507 | 1.99 |
| | | yl)methyl | | | | (3) |
| 52 | 25 (66A) | (1-methyl-1H- | 3-(trifluoro- | 83 | 558 | 1.77 |
| | | imidazol-2-yl)- | methyl)phenoxy | | | (1) |
| | | methyl | | | | |
| 53 | 25 (67A) | (1-methyl-1H- | 4-(trifluoro- | 93 | 558 | 1.77 |
| | | imidazol-2- | methyl)phenoxy | | | (1) |
| | | yl)methyl | | | | |
| 54 | 25 (68A) | 2-methoxyethyl | 3-(trifluoro- | 93 | 522 | 2.80 |
| | | | methyl)phenoxy | | | (2) |
| 55 | 25 (69A) | (3,5-dimethyl- | 4-methylphenoxy | 35 | 519 | 2.72 |
| | | isoxazol-4-yl)- | | | | (3) |
| | | methyl | | | | |
| 56 | 25 (70A) | (2,4-dimethyl- | 3-(trifluoro- | 73 | 589 | 2.63 |
| | | 1,3-thiazol-5-yl)- | methyl)phenoxy | | | (1) |
| | | methyl | | | | |
| 57 | 25 (71A) | (2,4-dimethyl- | 4-(trifluoro- | 90 | 589 | 2.64 |
| | | 1,3-thiazol-5-yl)- | methyl)phenoxy | | | (1) |
| | | methyl | | | | |
| 58 | 25 (72A) | (2,4-dimethyl- | 3,4-difluoro- | 81 | 557 | 2.67 |
| | | 1,3-thiazol-5-yl)- | phenoxy | | | (3) |
| | | methyl | | | | |
| 59 | 25 (73A) | (2,4-dimethyl- | 3,5-difluoro- | 79 | 557 | 2.72 |
| | | 1,3-thiazol-5-yl)- | phenoxy | | | (3) |
| | | methyl | | | | |
| 60 | 25 (74A) | (2,4-dimethyl- | 3-chlorophenoxy | 92 | 555 | 2.76 |
| | | 1,3-thiazol-5-yl)- | | | | (3) |
| | | methyl | | | | |
| 61 | 25 (75A) | (2,4-dimethyl- | 3-methylphenoxy | 71 | 535 | 2.67 |
| | | 1,3-thiazol-5-yl)- | | | | (3) |
| | | methyl | | | | |
| 62 | 25 (76A) | 2-methoxyethyl | 4-(trifluoro- | 98 | 506 | 2.36 |
| | | | methyl)phenyl | | | (6) |
| 63 | 25 (77A) | 2-methoxyethyl | 4-(trifluoro- | 98 | 522 | 2.43 |
| | | | methoxy)phenyl | | | (1) |
| 64 | 25 (78A) | 2-methoxyethyl | 3-(trifluoro- | 95 | 522 | 2.50 |
| | | | methoxy)phenyl | | | (1) |
| 65 | 27 (79A) | 2-methoxyethyl | 4-fluoro-3- | 68 | 470 | 2.28 |
| | | | methylphenyl | | | (3) |
| 66 | 25 (80A) | (2-methyl-1,3- | 3-(trifluoro- | 89 | 559 | 2.77 |
| | | thiazol-4-yl)- | methyl)phenyl | | | (3) |
| | | methyl | | | | |
| 67 | 25 (81A) | (2-methyl-1,3- | 4-(trifluoro- | 94 | 559 | 2.73 |
| | | thiazol-4-yl)- | methyl)phenyl | | | (2) |
| | | methyl | | | | |
| 68 | 25 (82A) | (1-methyl-1H- | 3-(trifluoro- | 87 | 542 | 1.73 |
| | | imidazol-2-yl)- | methyl)phenyl | | | (1) |
| | | methyl | | | | |
| 69 | 25 (83A) | (1-methyl-1H- | 4-(trifluoro- | 96 | 542 | 1.69 |
| | | imidazol-2-yl)- | methyl)phenyl | | | (1) |
| | | methyl | | | | |
| 70 | 25 (84A) | (2,4-dimethyl- | 4-(trifluoro- | 91 | 573 | 2.80 |
| | | 1,3-thiazol-5-yl)- | methyl)phenyl | | | (3) |
| | | methyl | | | | |
| 71 | 25 (85A) | (2,4-dimethyl- | 3-(trifluoro- | 95 | 573 | 2.76 |
| | | 1,3-thiazol-5-yl)- | methyl)phenyl | | | (2) |
| | | methyl | | | | |
| 72 | 25 (86A) | (2,4-dimethyl- | 4-methylphenyl | 80 | 519 | 2.12 |
| | | 1,3-thiazol-5-yl)- | | | | (1) |
| | | methyl | | | | |
| 73 | 25 (87A) | cyclopropyl- | 3-(trifluoro- | 61 | 502 | 2.62 |
| | | methyl | methyl)phenyl | | | (1) |

*with direct isolation of the acid

### B. ASSESSMENT OF THE PHARMACOLOGICAL ACTIVITY

The pharmacological activity of the compounds according to the invention can be demonstrated by the following assays:

### 1. Cellular Transactivation Assay:

### a) Test Principle:

A cellular assay is used to identify activators of the peroxysome proliferator-activated receptor alpha (PPAR-alpha).

Since mammalian cells contain different endogenous nuclear receptors which may complicate an unambiguous interpretation of the results, an established chimera system is used in which the ligand binding domain of the human PPARα receptor is fused to the DNA binding domain of the yeast transcription factor GAL4. The resulting GAL4-PPARα chimera is co-transfected and stably expressed in CHO cells having a reporter construct.

### b) Cloning:

The GAL4PPARα expression construct contains the ligand binding domain of PPARα (amino acids 167-468) which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARα.

c) Transactivation Assay (Luciferase Reporter):

CHO (Chinese hamster ovary) cells are sown in DMEM/F12 medium (BioWhittaker) supplemented by 10% fetal calf serum and 1% penicillin/streptomycin (GIBCO), at a cell density of 2×10³ cells per well in a 384-well plate (Greiner). The cells are cultivated at 37° C. for 48 h and then stimulated. To this end, the substances to be tested are taken up in CHO-A-SFM medium (GIBCO) supplemented by 10% fetal calf serum and 1% penicillin/streptomycin (GIBCO) and added to the cells. After a stimulation period of 24 hours, the luciferase activity is measured using a video camera. The relative light units measured give, as a function of the substance concentration, a sigmoidal stimulation curve. The EC₅₀ values are calculated using the computer program GraphPad PRISM (Version 3.02).

In this test, the compounds of the invention show EC₅₀ values of from 1 μM to 1 nM.

### 2. Fibrinogen Determination:

To determine the effect on the plasma fibrinogen concentration, male Wistar rats or NMRI mice are treated with the substance to be examined by stomach tube administration or by addition to the feed for a period of 4-9 days. Under terminal anesthesia, citrate blood is then obtained by heart puncture. The plasma fibrinogen concentrations are determined according to the Clauss method [A. Clauss, Acta Haematol. 17, 237-46 (1957)] by measuring the thrombin time using human fibrinogen as standard.

3. Description of a test for finding pharmacologically active substances which increase apoprotein A1 (ApoA1) and HDL cholesterol (HDL-C) concentrations in the serum of transgenic mice transfected with the human ApoA1 gene (hApoA1) and/or lower serum triglycerides (TG):

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+ saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the final administration of substance (on the 8^{th} day after the beginning of treatment), a blood sample from each of the animals is again taken by puncture of the retroorbital venous plexus, to determine the same parameters. The blood samples are centrifuged and, after the serum has been obtained, TG, cholesterol, HDL-C and human ApoA1 are determined using a Cobas Integra 400 plus instrument (Cobas Integra, Roche Diagnostics GmbH, Mannheim, Germany) using the respective cassettes (TRIGL, CHOL2, HDL-C and APOAT). HDL-C is determined by gel filtration and post-column derivatization with MEGA cholesterol reagent (Merck KGaA) analogously to the method of Garber et al. [J. Lipid Res. 41 1020-1026 (2000)].

The effect of the test substances on HDL-C, hApoA1 and TG concentrations is determined by subtracting the value measured for the first blood sample (zero value) from the value measured for the second blood sample (after the treatment). The means of the differences of all HDL-C, hApoA 1 and TG values of a group are determined and compared with the mean of the differences of the control group. Statistical evaluation is carried out using Student's t-Test, after the variances have been checked for homogeneity.

Substances which increase the HDL-C of the treated animals, compared to that of the control group, in a statistically significant (p<0.05) manner by at least 20% or which lower TG in a statistically significant (p<0.05) manner by at least 25% are considered to be pharmacologically effective.

### C. WORKING EXAMPLES OF PHARMACEUTICAL COMPOSITIONS

The Compounds According to The Invention can be Converted Into Pharmaceutical Preparations in the following ways:

### Tablet:

### Composition:

100 mg of the compound of the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of the compound of the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

### Composition:

500 mg of the compound of the invention, 2.5 g of polysorbate and 97 g of polyethylene glycol 400.20 g of oral solution correspond to a single dose of 100 mg of the compound of the invention.

### Production:

The compound of the invention is suspended in the mixture of polyethylene glycol and polysorbate with stirring. Stirring is continued until the compound of the invention has dissolved completely.

I.V. Solution:

The compound of the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. A compound of the formula (I) 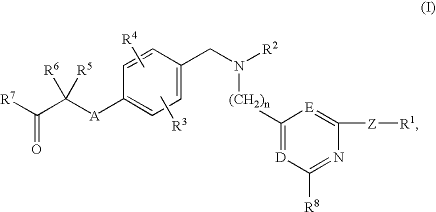
in which
A represents O or S,
one of the ring members D and E represents N and the other represents CH,
Z represents (CH₂)ₘ, O or N—R⁹, where
m represents the number 0, 1 or 2,
and
R⁹ represents hydrogen or (C₁-C₆)-alkyl,
n represents the number 0, 1 or 2,
R¹ represents (C₆-C₁₀)-aryl or 5- to 10-membered heteroaryl which may in each case be substituted up to four times by identical or different substituents selected from the group consisting of halogen, nitro, cyano, (C₁-C₆)-alkyl (which for its part may be substituted by hydroxyl), (C₃-C₈)-cycloalkyl, phenyl, hydroxyl, (C₁-C₆)-alkoxy, trifluoromethyl, trifluoromethoxy, amino, mono- and di-(C₁-C₆)-alkylamino, R¹⁰—C(O)—NH—, R¹¹—C(O)—, R¹²R¹³N—C(O)—NH— and R¹⁴R¹⁵N—C(O)—, where
R¹⁰ represents hydrogen, (C₁-C₆)-alkyl, (C₃-C₈)-cycloalkyl, phenyl or (C₁-C₆)-alkoxy,
R¹¹ represents hydrogen, (C₁-C₆)-alkyl, (C₃-C₈)-cycloalkyl, phenyl, hydroxyl or (C₁-C₆)-alkoxy
and
R¹², R¹³, R¹⁴ and R¹⁵ are identical or different and independently of one another represent hydrogen, (C₁-C₆)-alkyl, (C₃-C₈)-cycloalkyl or phenyl,
or
R¹ represents (C₃-C₇)-cycloalkyl or a 5- or 6-membered heterocycle which may in each case be substituted up to two times by identical or different substituents from the group consisting of (C₁-C₆)-alkyl, (C₁-C₆)-alkoxy, trifluoromethyl or trifluoromethoxy,
or
the grouping -Z-R¹ represents a group of the formula 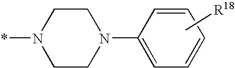
in which
R¹⁸ represents hydrogen, halogen, (C₁-C₆)-alkyl, (C₁-C₆)-alkoxy, trifluoromethyl or trifluoromethoxy
and
* represents the point of attachment,
R² represents hydrogen, (C₆-C₁₀)-aryl, (C₁-C₆)-alkyl, (C₂-C₆)-alkenyl or (C₂-C₆)-alkynyl, where alkyl, alkenyl and alkynyl may in each case be substituted by trifluoromethyl, (C₁-C₆)-alkoxy, trifluoromethoxy, fluorine, cyano, (C₃-C₆)-cycloalkyl, (C₆-C₁₀)-aryl or 5- or 6-membered heteroaryl, where all aryl and heteroaryl groups mentioned for their part may be substituted up to three times by identical or different substituents selected from the group consisting of halogen, nitro, cyano, (C₁-C₆)-alkyl, hydroxyl, (C₁-C₆)-alkoxy, trifluoromethyl and trifluoromethoxy,
R³ and R⁴ are identical or different and independently of one another represent hydrogen, (C₁-C₆)-alkyl, (C₂-C₆)-alkenyl, (C₁-C₆)-alkoxy, trifluoromethyl, trifluoromethoxy or halogen,
—R⁵ and R⁶ are identical or different and independently of one another represent hydrogen, (C₁-C₆)-alkyl, (C₁-C₆)-alkoxy or phenoxy or together with the carbon atom to which they are attached form a (C₃-C₈)-cycloalkyl ring,
R⁷ represents a group of the structure —NHR¹⁶ or —OR¹⁷, in which
R¹⁶ represents hydrogen, (C₁-C₆)-alkyl or (C₁-C₆)-alkylsulfonyl
and
R¹⁷ represents hydrogen or represents a hydrolysable group which can be converted into the corresponding carboxylic acid,
and
R⁸ represents hydrogen or (C₁-C₆)-alkyl,
or a salt, a solvate or a solvate of a salt thereof.

2. The compound of the formula (I) as claimed in claim 1 in which
A represents O or S,
one of the ring members D and E represents N and the other represents CH,
Z represents (CH₂)ₘ, O or NH, where
m represents the number 0 or 1,
n represents the number 0 or 1,
R¹ represents phenyl or 5- or 6-membered heteroaryl which may in each case be substituted up to four times by identical or different substituents selected from the group consisting of halogen, nitro, cyano, (C₁-C₄)-alkyl (which for its part may be substituted by hydroxyl), (C₃-C₆)-cycloalkyl, phenyl, hydroxyl, (C₁-C₄)-alkoxy, trifluoromethyl, trifluoromethoxy, amino, mono- and di-(C₁-C₄)-alkylamino, R¹⁰—C(O)—NH—, R¹¹—C(O)—, R¹²R¹³N—C(O)—NH— and R¹⁴R¹⁵N—C(O)—, where
R¹⁰ represents hydrogen, (C₁-C₄)-alkyl, (C₃-C₆)-cycloalkyl, phenyl or (C₁-C₄)-alkoxy,
R¹¹ represents hydrogen, (C₁-C₄)-alkyl, (C₃-C₆)-cycloalkyl, phenyl, hydroxyl or (C₁-C₄)-alkoxy
and
R¹², R¹³, R¹⁴ and R¹⁵ are identical or different and independently of one another represent hydrogen, (C₁-C₄)-alkyl, (C₃-C₆)-cycloalkyl or phenyl,
or
R¹ represents cyclohexyl or 4-tetrahydropyranyl which may in each case be substituted up to two times by identical or different substituents from the group consisting of (C₁-C₄)-alkyl, (C₁-C₄)-alkoxy and trifluoromethyl,
R² represents hydrogen, phenyl, (C₁-C₄)-alkyl, (C₂-C₄)-alkenyl or (C₂-C₄)-alkynyl, where alkyl, alkenyl and alkynyl may in each case be substituted by trifluoromethyl, fluorine, cyano, (C₁-C₄)-alkoxy, cyclopropyl, cyclobutyl, phenyl or a 5- or 6-membered heteroaryl, where all phenyl and heteroaryl groups mentioned for their part may in each case be substituted up to three times by identical or different substituents selected from the group consisting of halogen, nitro, cyano, (C₁-C₄ alkyl, hydroxyl, (C₁-C₄)-alkoxy, trifluoromethyl and trifluoromethoxy,
R³ and R⁴ are identical or different and independently of one another represent hydrogen, (C₁-C₄)-alkyl, (C₁-C₄)-alkoxy, trifluoromethyl, trifluoromethoxy or halogen,
R⁵ and R⁶ are identical or different and independently of one another represent hydrogen, methyl, ethyl, methoxy, ethoxy or phenoxy or together with the carbon atom to which are attached form a (C₃-C₆)-cycloalkyl ring,
R⁷ represents a group of the formula —NHR¹⁶ or —OR¹⁷, in which
R¹⁶ represents hydrogen or (C₁-C₄)-alkyl
and
R¹⁷ represents hydrogen or represents a hydrolysable group which may be converted into the corresponding carboxylic acid, and
R⁸ represents hydrogen or methyl,
or a salt, a solvate or a solvate of a salt thereof.

3. The compound of the formula (I) as claimed in claim 1 or 2 in which
A represents S,
one of the ring members D and E represents N and the other represents CH,
Z represents (CH₂)ₘ, O or NH, where
m represents the number 0 or 1,
n represents the number 0 or 1,
R¹ represents phenyl or pyridyl which may in each case be mono- or disubstituted by identical or different substituents from the group consisting of fluorine, chlorine, nitro, methyl, methoxy, trifluoromethyl and trifluoromethoxy
or
R¹ represents cyclohexyl which may be substituted in the 4-position by methyl or methoxy,
R² represents hydrogen, propargyl or represents (C₁-C₄)-alkyl which may be substituted by fluorine, cyano, (C₁-C₄)-alkoxy, cyclopropyl, phenyl, furyl, thienyl, imidazolyl, oxazolyl, isoxazolyl, thiazolyl, oxadiazolyl or thiadiazolyl, where phenyl and all heteroaromatic rings mentioned for their part may in each case be mono- or disubstituted by identical or different substituents selected from the group consisting of fluorine, chlorine, methyl, ethyl, isopropyl, tert-butyl, methoxy, ethoxy, trifluoromethyl and trifluoromethoxy,
R³ and R⁴ are identical or different and independently of one another represent hydrogen, methyl, methoxy, fluorine or chlorine,
R⁵ and R⁶ are identical or different and represent hydrogen or methyl,
R⁷ represents —OH, —NH₂ or —NHCH₃, and
R⁸ represents hydrogen,
a salt, a solvate or a solvate of a salt thereof.

4. A compound of the formula (I-A) 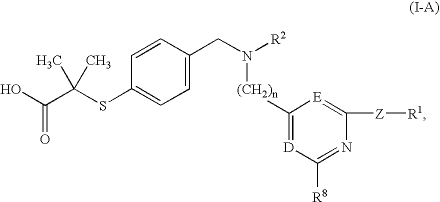
in which
R¹, R², R⁸, D, E, Z and n are each as defined in claims 1 to 3,
or a salt, a solvate or a solvate of a salt thereof.

5. A compound of the formula (I-C) 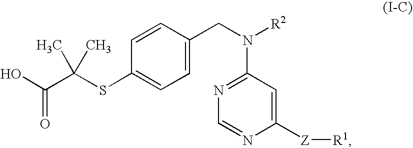
in which
Z represents a bond or represents O
and
R¹ and R² are each as defined in claims 1 to 3,
or a salt, a solvate or a solvate of a salt thereof.

6. A process for preparing a compound of the formula (I), (I-A) or (I-C) as defined in claims 1 to 5, characterized in that compounds of the formula (II) 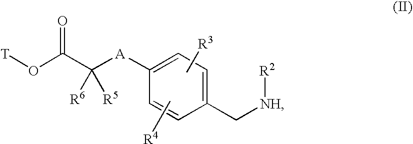
in which R², R³, R⁴, R⁵, R⁶ and A are each as defined in claims 1 to 5
and
T represents (C₁-C₄)-alkyl, preferably tert-butyl, or represents benzyl,
are either
[A] initially reacted in an inert solvent in the presence of a base with a compound of the formula (III) 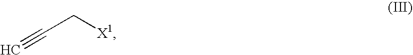
in which
X¹ represents a suitable leaving group, such as, for example, halogen,
to give compounds of the formula (IV) 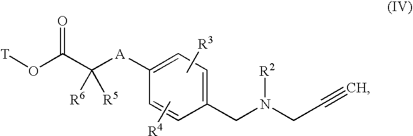
in which A, T, R², R³, R⁴, R⁵ and R⁶ are each as defined above,
then converted, in an inert solvent in the presence of copper(I) iodide, a suitable palladium catalyst and a base, with a compound of the formula (V) 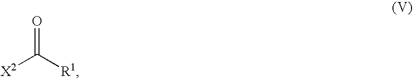
in which R¹ is as defined in claims 1 to 5 and
X² represents a suitable leaving group, such as, for example, halogen, into compounds of the formula (VI) 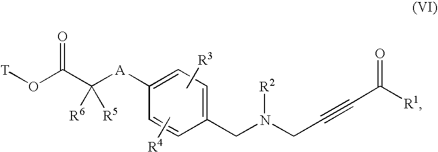
in which A, T, R¹, R², R³, R⁴, R⁵ and R⁶ are each as defined above,
which compounds are then reacted, in an inert solvent in the presence of a base, with a compound of the formula (VII) 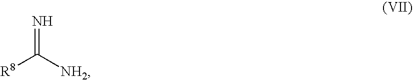
in which R⁸ is as defined in claims 1 to 5,
to give compounds of the formula (VIII) 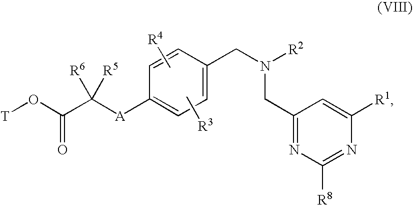
in which A, T, R¹, R², R³, R⁴, R⁵, R⁶ and R⁸ are each as defined above,
or
[B] initially converted, in an inert solvent in the presence of a base, with a compound of the formula (IX) 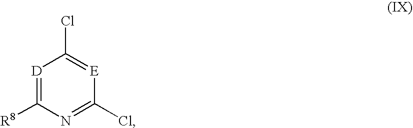
in which D, E and R⁸ are each as defined in claims 1 to 5,
into compounds of the formula (X) 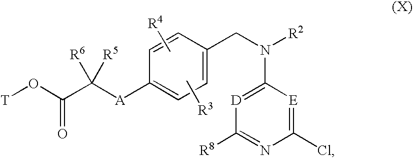
in which A, D, E, T, R², R³, R⁴, R⁵, R⁶ and R⁸ are each as defined above,
and these compounds are then either
[B-1]
reacted, in an inert solvent in the presence of a base, with a compound of the formula (XI)
R¹-Z¹-H (XI),
in which R¹ is as defined in claims 1 to 5 and
Z¹ represents O or N—R⁹, where R⁹ is as defined in claims 1 to 4,
to give compounds of the formula (XII) 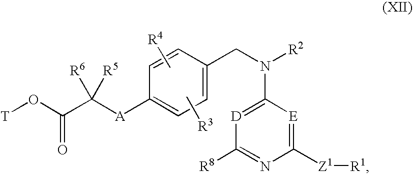
in which A, D, E, T, Z¹, R¹, R², R³, R⁴, R⁵, R⁶ and R⁸ are each as defined above,
or
[B-2]
reacted, in an inert solvent in the presence of a palladium catalyst and a base, with a compound of the formula (XIII) 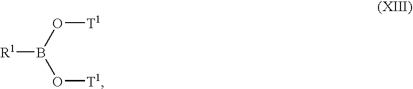
in which R¹ is as defined in claims 1 to 5 and
T¹ represents hydrogen or (C₁-C₄)-alkyl,
to give compounds of the formula (XIV) 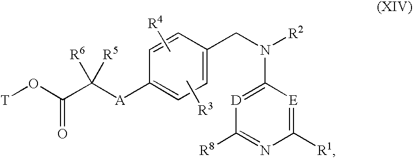
in which A, D, E, T, R¹, R², R³, R⁴, R⁵, R⁶ and R⁸ are each as defined above,
or
[B-3]
reacted, in an inert solvent in the presence of a palladium catalyst, with a compound of the formula (XV) 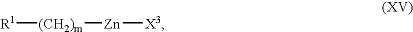
in which m and R¹ are each as defined in claims 1 to 5 and
X³ represents halogen, in particular bromine,
to give compounds of the formula (XVI) 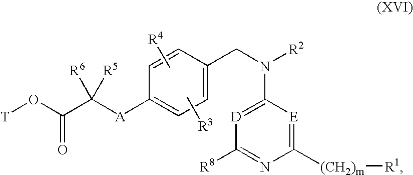
in which m, A, D, E, T, R¹, R², R³, R⁴, R⁵, R⁶ and R⁸ are each as defined above,
or
[C] reacted, in an inert solvent in the presence of a base, with a compound of the formula (XVII) 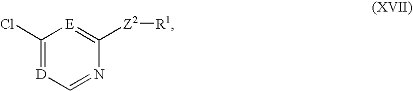
in which D, E and R¹ are each as defined in claims 1 to 5 and
Z² represents a bond, O or N—R⁹, where R⁹ is as defined in claims 1 to 4,
to give compounds of the formula (XVII) 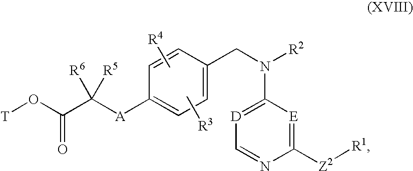
in which A, D, E, T, Z², R¹, R², R³, R⁴, R⁵ and R⁶ are each as defined above,
and the resulting compounds of the formulae (VIII), (XII), (XIV), (XVI) and (XVIII) are subsequently converted by basic or acidic hydrolysis or, if T represents benzyl, also hydrogenolytically, into the respective carboxylic acids of the formula (I-B) 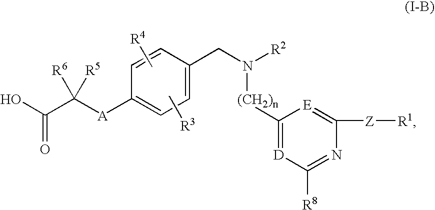
in which n, A, D, E, Z, R¹, R², R³, R⁴, R⁵, R⁶ and R⁸ are each as defined above,
and, if appropriate, subsequently converted into the compounds of the formula (I) using esterification or amidation methods known from the literature,
and the compounds of the formula (I) are, if appropriate, reacted with the appropriate (i) solvents and/or (ii) bases or acids to give their solvates, salts and/or solvates of the salts.

7. The compound as defined in any of claims 1 to 5 for the treatment and/or prophylaxis of diseases.

8. The use of a compound as defined in any of claims 1 to 5 for preparing a medicament for the treatment and/or prevention of dyslipidemias, arteriosclerosis, coronary heart disease, thrombosis and metabolic syndrome.

9. A medicament, comprising a compound as defined in any of claims 1 to 5 in combination with an inert non-toxic pharmaceutically suitable auxiliary.

10. A medicament, comprising the compound as defined in any of claims 1 to 5 in combination with a further active compound selected from the group consisting of PPAR-gamma and/or PPAR-delta agonists, CETP inhibitors, thyroid hormones and/or thyroid mimetics, inhibitors of HMG-CoA reductase, inhibitors of HMG-CoA reductase expression, squalene synthesis inhibitors, ACAT inhibitors, cholesterol absorption inhibitors, bile acid absorption inhibitors, MTP inhibitors, niacin receptor agonists, aldolase reductase inhibitors, lipase inhibitors, antidiabetics, antioxidants, calcium antagonists, angiotensin-II receptor antagonists, ACE inhibitors, alpha-receptor blockers, beta-receptor blockers, platelet aggregation inhibitors, anticoagulants, profibrinolytic substances, anorectics and cytostatics.

11. The medicament as claimed in claim 9 or 10 for the treatment and/or prevention of dyslipidemias, arteriosclerosis, coronary heart disease, thrombosis and metabolic syndrome.

12. A method for the treatment and/or prevention of dyslipidemias, arteriosclerosis, coronary heart disease, thrombosis and metabolic syndrome in humans and animals by administering an effective amount of at least one compound as defined in any of claims 1 to 5 or a medicament as defined in any of claims 9 to 11.
